(19) United States
(12) Patent Application Publication
(10) Pub. No.: US 2004/0219214 A1
(43) Pub. Date: Nov. 4, 2004

(54) Tissue reactive compounds and compositions and uses thereof

(75) Inventors: David M. Gravett, Vancouver (CA); Aniko Takacs-Cox, North Vancouver (CA); Philip M. Toleikis, Vancouver (CA); Arpita Maiti, Vancouver (CA); Leanne Embree, Squamish (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 10/749,123

(22) Filed: Dec. 30, 2003

Related U.S. Application Data
(60) Provisional application No. 60/440,924, filed on Jan. 17, 2003.
(60) Provisional application No. 60/437,384, filed on Dec. 30, 2002.

(51) Int. Cl. A61K 9/14

(52) U.S. Cl. 424/484

(57) ABSTRACT

A composition comprising a synthetic polymer, optionally in the presence of a drug, where the polymer comprises multiple activated groups. The multiple activated groups are reactive with functionality present on animal tissue, so that upon administration of the polymer to the tissue, the polymer binds to the tissue. Alternatively, the multiple activated groups are reactive with functionality present on a non-living surface, where the polymer binds to this surface to, e.g., increase the lubricity of the surface. When drug is present in the composition, the drug is then delivered to the site of polymer attachment.

### CROSS REFERENCE TO RELATED APPLICATIONS

This application claims the benefit of U.S. Provisional Patent Application No. 60/437,384, filed Dec. 30, 2002, and U.S. Provisional Patent Application No. 60/440,924, filed Jan. 17, 2003, where these provisional applications are incorporated herein by reference in their entireties.

### BACKGROUND OF THE INVENTION

1. Field of the Invention

This invention relates generally to compositions comprising a synthetic polymer that contains multiple activated groups and methods of using such compositions in medical applications as well as in device applications.

2. Description of the Related Art

U.S. Pat. No. 5,162,430, issued Nov. 10, 1992, to Rhee et al., and commonly owned by the assignee of the present invention, discloses collagen-synthetic polymer conjugates prepared by covalently binding collagen to synthetic hydrophilic polymers such as various derivatives of polyethylene glycol.

U.S. Pat. No. 5,324,775, issued Jun. 28, 1994, to Rhee et al., discloses various insert, naturally occurring, biocompatible polymers (such as polysaccharides) covalently bound to synthetic, non-immunogenic, hydrophilic polyethylene glycol polymers.

U.S. Pat. No. 5,328,955, issued Jul. 12, 1994, to Rhee et al., discloses various activated forms of polyethylene glycol and various linkages which can be used to produce collagen-synthetic polymer conjugates having a range of physical and chemical properties.

U.S. application Ser. No. 08/403,358, filed Mar. 14, 1995, discloses a crosslinked biomaterial composition that is prepared using a hydrophobic crosslinking agent, or a mixture of hydrophilic and hydrophobic crosslinking agents. Preferred hydrophobic crosslinking agents include any hydrophobic polymer that contains, or can be chemically derivatized to contain, two or more succinimidyl groups.

U.S. application Ser. No. 08/403,360, filed Mar. 14, 1995, discloses a composition useful in the prevention of surgical adhesions comprising a substrate material and an anti-adhesion binding agent, where the substrate material preferably comprises collagen and the binding agent preferably comprises at least one tissue-reactive functional group and at least one substrate-reactive functional group.

U.S. application Ser. No. 08/476,825, filed Jun. 7, 1995, by Rhee et al., discloses bioadhesive compositions comprising collagen crosslinked using a multifunctionally activated synthetic hydrophilic polymer, as well as methods of using such compositions to effect adhesion between a first surface and a second surface, wherein at least one of the first and second surfaces is preferably a native tissue surface.

Japanese patent publication No. 07090241 discloses a composition used for temporary adhesion of a lens material to a support, to mount the material on a machining device, comprising a mixture of polyethylene glycol, having an average molecular weight in the range of 1000-5000, and poly-N-vinylpyrrolidone, having an average molecular weight in the range of 30,000-200,000.

West and Hubbell, Biomaterials (1995) 16:1153-1156, disclose the prevention of post-operative adhesions using a photopolymerized polyethylene glycol-co-lactic acid diacrylate hydrogel and a physically crosslinked polyethylene glycol-co-polypropylene glycol hydrogel, Poloxamer 407 (BASF Corporation, Mount Olive, N.J.).

U.S. Pat. No. 5,874,500, U.S. Pat. No. 6,051,648 and U.S. Pat. No. 6,312,725 disclose the in-situ crosslinking or crosslinked polymers. These disclosures describe the use of synthetic polymers, in particular poly(ethylene glycol) based polymers, to produce the crosslinked composition.

### BRIEF SUMMARY OF THE INVENTION

Briefly stated, the present invention provides compositions that are reactive with surfaces, particularly in vivo surfaces such as tissue, but also the surface of a medical device. Various beneficial goals are achieved by having the synthetic polymer react with the surface. The compositions may or may not include a drug.

For example, in one aspect the present invention provides a composition comprising a) a synthetic polymer comprising multiple activated groups; and b) an aqueous buffer; wherein the composition is a homogeneous solution having a pH of less than 6. In a related aspect, the present invention provides a composition comprising a) a synthetic polymer comprising multiple activated groups; and b) an aqueous buffer; wherein the composition is a homogeneous solution having a pH of greater than about 7.8. Preferred synthetic polymers having multiple activated groups are described below. In either of these aspects of the invention, in various optional embodiments it may further be stated, for example, that: the composition does not contain any polymer that is reactive with the synthetic polymer; and/or the composition further comprises a drug; the composition further comprises a hydrophobic drug; the composition further comprises a hydrophilic drug, the composition further comprises a hydrophobic or hydrophilic drug is association with a secondary carrier, e.g., a secondary carrier in the form of a micelle, microsphere or nanosphere; and/or the synthetic polymer comprises alkylene oxide residues; and/or the synthetic polymer comprises thiol-reactive groups; and/or the synthetic polymer comprises N-oxysuccinimidyl groups; and/or the the synthetic polymer is one of the 4-arm PEG polymers describe herein; and/or the composition are sterile. These and other embodiments of this aspect of the present invention are described in further detail below.

In related aspects, the present invention provides a method for preparing a reactive composition, the method comprising a) providing a synthetic polymer comprising multiple activated groups; b) combining the synthetic polymer with a buffer having a pH of less than 6 to form a homogeneous solution; and c) raising the pH of the homogeneous solution to a pH of more than about 7.8, thereby rendering the synthethic polymer reactive. In addition, the present invention provides a method whereby the reactive synthetic polymer is reacted with tissue. In this aspect, the present invention provides a method of adhering a synthetic polymer to in vivo tissue, where the method comprises a) providing a synthetic polymer comprising multiple activated groups; b) combining the synthetic polymer with a buffer having a pH of less than 6 to form a homogeneous solution; c) raising the pH of the homogeneous solution to a pH of more than about 7.8, thereby rendering the synthethic polymer reactive; and d) contacting the reactive synthetic polymer with in vivo tissue.

The present invention further provides a method of coating a device comprising: a) applying a multifunctional hydroxysuccinimidyl PEG derivative to the surface of the device; and b) allowing the derivative to react with functional groups on the device surface. In certain embodiments, the functional surface groups on the device are incorporated into the device using a surface treatment process (e.g., a plasma treatment process or a surface treatment process that includes coating the surface of the device with a polymer having functional groups (e.g., amino groups) that can react with the mulitfunctional hydroxysuccinimidyl PEG derivative. Representaive examples of such polymers include chitosan and polyethyleneimine. In one aspect, the multifunctional hydroxysuccinimidyl PEG derivative is tetra functional poly(ethylene glycol) succinimidyl glutarate.

Optionally, the synthetic polymer is combined with a drug, e.g., a hydrophobic drug, where the drug is optionally in association with a secondary carrier, and the secondary carrier is dispersed in aqueous media. This and other optional embodiments of these aspects of the present invention are described in further detail herein. However, in brief summary, some of these optional embodiments are, without limitation: the synthetic polymer comprises alkylene oxide residues; the synthetic polymer comprises thiol-reactive groups; the synthetic polymer comprises N-oxysuccinimidyl groups; the synthetic polymer is contacted with the tissue prior to raising the pH of the homogeneous solution to a pH of more than about 7.8; and the synthetic polymer is contacted with the tissue after raising the pH of the homogeneous solution to a pH of more than about 7.8.

The compositions of the present invention may be utilized in various methods. For example, in one aspect, the present invention provides a method comprising a) contacting tissue in vivo with a synthetic polymer comprising multiple activated groups, where the activated groups are tissue-reactive; and b) reacting the synthetic polymer with the tissue so as to covalently adhere the synthetic polymer to the tissue. In a related aspect, the present invention provides a method comprising a) contacting a non-living surface with a synthetic polymer comprising multiple activated groups, where the activated groups are tissue-reactive; and b) reacting the synthetic polymer with the surface so as to covalently adhere the synthetic polymer to the surface. When the composition is contacted with tissue, some exemplary tissues include, without limitation, blood vessel and tissue prone to restenosis. The addition of the synthetic polymer to the tissue is advantageous, e.g., in instances where it is desirable that adhesion of the tissue to secondary tissue is mitigated.

When the composition is contacted with a non-living surface, that surface may be a surface of a medical device, e.g., a catheter or a contact lens. In either aspect, in various optional embodiments, the surface (tissue or non-living) is preferably not reacted with any other synthetic polymer; and/or the synthetic polymer is not in admixture with any other polymer that is reactive with the synthetic polymer; and/or the synthetic polymer is not in admixture with any other polymer that is reactive with the surface. Exemplary synthetic polymers are described in detail herein. However, in brief summary, in various optional embodiments of the invention, the synthetic polymer may be characterizered as comprising alkylene oxide residues; and/or the synthetic polymer is a 4-arm PEG as described herein; and/or the synthetic polymer comprises a plurality of thiol-reactive groups and/or a plurality of hydroxyl-reactive groups and/or a plurality of amine-reactive groups.

In preferred aspects of the invention, compositions and methods for drug delivery are provided, where these compositions and methods include synthetic polymers comprising multiple activated groups. Thus, in one aspect, the present invention provides a composition comprising a synthetic polymer and a drug, the polymer comprising multiple activated groups.

In these aspects of the invention that entail drug delivery, the compositions may be characterized by one or more optional features as described more fully herein. However, in brief summary, some of those optional features include (without limitation): the synthetic polymer has a cyclic core, e.g., a cyclic core that comprises a six-membered carbocyclic group, or a cyclic core that comprises an inositol, lactitol residue or sorbitol residue; the synthetic polymer has a branched chain core; the synthetic polymer has a branched chain core that is a polyhydric compound residue; the synthetic polymer has a branched chain core that is a glycerol residue; the synthetic polymer has a branched chain core that is a pentaerythritol residue; the synthetic polymer has a branched chain core that is a diglycerol residue; the synthetic polymer has a branched chain core that is a poly(carboxylic acid) compound residue; the synthetic polymer has a branched chain core that is a polyamine compound residue; or the synthetic polymer has a branched chain core that comprises polyamino acid.

In other optional embodiments: the synthetic polymer comprises poly(alkylene)oxide, the synthetic polymer comprises ethylene oxide residues; the synthetic polymer comprises propylene oxide residues. The synthetic polymer has a molecular weight that may be characterized as, e.g., a molecular weight of about 100 to about 100,000; a molecular weight of about 1,000 to about 20,000; a molecular weight of about 1,000 to about 15,000; a molecular weight of about 1,000 to about 10,000; a molecular weight of about 1,000 to about 5,000; a molecular weight of about 7,500 to about 20,000; a molecular weight of about 7,500 to about 15,000; a molecular weight of about 7,500 to about 20,000. The molecular weight may be number average molecular weight. The molecular weight may be weight average molecular weight.

In other optional embodiments: the synthetic polymer has 2-12 activated groups; for example, has 2 activated groups; or has 3 activated groups; or has 4 activated groups; or has 6 activated groups; or has 9 activated groups; or has 12 activated groups. Optionally, but preferably in those instances where the synthetic polymer is tissue reactive, the activated groups of the sythetic polymer are: protein-reactive; are reactive with hydroxyl groups; are reactive with thiol groups; are reactive with amino groups. As regards the chemical nature of the activated groups, in various optional embodiments, those groups may be characterized as: comprising an electrophilic site; being a carbonyl group; comprising a leaving group, where the leaving group is optionally an N-oxysuccinimide group or an N-oxymaleimide group; optionally the activated group comprises an electrophilic site adjacent to a leaving group; the electrophilic site is a carbonyl group; the leaving group is selected from N-oxysuccinimide and N-oxymaleimide; the electrophilic group is carbonyl and the leaving group is selected from N-oxysuccinimide and N-oxymaleimide.

The synthetic polymer comprising multiple activated groups may contain other moieties as discussed in greater detail below. For example, the synthetic polymer may comprise the formula (polymer backbone)-(Q-Y)ₙ wherein Q is a linking group, Y is an activated functional group, and n is an integer of greater than 1. Optionally, the polymer backbone comprises poly(alkylene) oxide; and/or Q is selected from the group consisting of -G-(CH₂)ₙ— wherein G is selected from O, S, NH, S—CO—, —O—CO— and —O—CO—NH—(CH₂)ₙ; O₂C—CR¹H— wherein R¹ is selected from hydrogen and alkyl; and O—R²—CO—NH wherein R² is selected from CH₂ and CO—NH—CH₂CH₂, where optionally n is 2-12; Y comprises an electrophilic cite adjacent to a leaving group, where optionally, the electrophilic site is a carbonyl group and optionally the leaving group comprises (N—CO—CH₂)₂.

As another example, the synthetic polymer may comprise the formula (polymer backbone)-(Q-Y)ₙ, where a chain extender is optionally located between either (polymer backbone) and Q or between Q and Y. For instance, the synthetic polymer may be characterized by the formula (polymer backbone)-(D-Q-Y)ₙ wherein D is a biodegradable group, Q is a linking group, Y is an activated functional group, and n is an integer of greater than 1. Optionally, D comprises a chemical group selected from lactide, glycolide, epsilon-caprolactone and poly(alpha-hydroxy acid), or D comprises a chemical group selected from poly(amino acid), poly(anhydride), poly(orthoester). Optionally, Q is selected from the group consisting of -G-(CH₂)ₙ— wherein G is selected from O, S, NH, —O—CO— and —O—CO—NH—(CH₂)ₙ; O₂C—CR¹H— wherein R¹ is selected from hydrogen and alkyl; and O—R²—CO—NH wherein R² is selected from CH₂ and CO—NH—CH₂CH₂.

In one aspect, the present invention provides a composition as briefly stated above, comprising first and second polymers comprising multiple activated groups, where the first and second polymers are non-identical. For example, the first and second polymer may comprise different activated groups; and/or the first and second polymers have different number average molecular weights; and/or the first and second polymers have a different number of activated groups.

The synthetic polymer comprising multiple active groups may be characterized by its physical properties. In one aspect of the invention, the synthetic polymer is soluble in water at a concentration of at least 1 grams polymer/99 grams water at 25° C.; while in another aspect the synthetic polymer is soluble in water at a concentration of at least 2 grams polymer/99 grams water at 25° C.; while in another aspect the synthetic polymer is soluble in water at a concentration of at least 3 grams polymer/99 grams water at 25° C.; while in another aspect the synthetic polymer is soluble in water at a concentration of at least 4 grams polymer/99 grams water at 25° C.; while in another aspect the synthetic polymer is soluble in water at a concentration of at least 5 grams polymer/99 grams water at 25° C.

In these aspects of the invention that include a drug, suitable drugs are described in great detail herein. However, briefly stated, in one optional aspect, the the drug is efficacious in inhibiting one or a combination of cellular activities selected from the group consisting of cell division, cell secretion, cell migration, cell adhesion, inflammatory activator production and/or release, angiogenesis and free radical formation and/or release. For example, the drug is an angiogenesis inhibitor; or a 5-Lipoxygenase inhibitor or antagonist; or a chemokine receptor antagonist; or a cell cycle inhibitor or an analogue or derivative thereof (e.g., a microtubule stabilizing agent, such as paclitaxel, docetaxel, or Peloruside A; a taxane, such as paclitaxel or an analogue or derivative thereof; an antimetabolite, an alkylating agent, or a vinca alkaloid (e.g., vinblastine, vincristine, vincristine sulfate, vindesine, vinorelbine, or an analogue or derivative thereof); camptothecin or an analogue or derivative thereof; mitoxantrone, etoposide, 5-fluorouracil, doxorubicin, methotrexate, Mitomycin-C, CDK-2 inhibitors, and analogues and derivatives thereof); or a cyclin dependent protein kinase inhibitor or an analogue or derivative thereof; or an EGF (epidermal growth factor) kinase inhibitor or an analogue or derivative thereof; or an elastase inhibitor or an analogue or derivative thereof; or a factor Xa inhibitor or an analogue or derivative thereof; or a farnesyltransferase inhibitor or an analogue or derivative thereof; or a fibrinogen antagonist or an analogue or derivative thereof; or a guanylate cyclase stimulant or an analogue or derivative thereof; or a heat shock protein 90 antagonist or an analogue or derivative thereof; or an HMGCOA reductase inhibitor or an analogue or derivative thereof; or a hydroorotate dehydrogenase inhibitor or an analogue or derivative thereof; or an IKK2 inhibitor or an analogue or derivative thereof; or an IL-1, ICE, or IRAK antagonist or an analogue or derivative thereof; or an IL-4 agonist or an analogue or derivative thereof; or an immunomodulatory agent (e.g., rapamycin, tacrolimus, everolimus, biolimus) or an analogue or derivative thereof; or an inosine monophosphate dehydrogenase inhibitor or an analogue or derivative thereof; or a leukotreine inhibitor or an analogue or derivative thereof; or a MCP-1 antagonist or an analogue or derivative thereof; or a MMP inhibitor or an analogue or derivative thereof; or a NF kappa B inhibitor or an analogue or derivative thereof; or a NO antagonist or an analogue or derivative thereof; or a P38 MAP kinase inhibitor or an analogue or derivative thereof; or a phosphodiesterase inhibitor or an analogue or derivative thereof; or a TGF beta Inhibitor or an analogue or derivative thereof; or a thromboxane A2 antagonist or an analogue or derivative thereof; or a TNFa Antagonist, a TACE, or an analogue or derivative thereof; or a tyrosine kinase inhibitor or an analogue or derivative thereof; or a vitronectin inhibitor or an analogue or derivative thereof; or a fibroblast growth factor inhibitor or an analogue or derivative thereof; or a protein kinase inhibitor or an analogue or derivative thereof; or a PDGF receptor kinase inhibitor or an analogue or derivative thereof; or an endothelial growth factor receptor kinase inhibitor or an analogue or derivative thereof; or a retinoic acid receptor antagonist or an analogue or derivative thereof; or a platelet derived growth factor receptor kinase inhibitor or an analogue or derivative thereof; or a fibrinogin antagonist or an analogue or derivative thereof; or an antimycotic agent or an analogue or derivative thereof; or a bisphosphonate or an analogue or derivative thereof; or a phospholipase A1 inhibitor or an analogue or derivative thereof; or a histamine H1/H2/H3 receptor antagonist or an analogue or derivative thereof; or a macrolide antibiotic or an analogue or derivative thereof; or an GPIIb IIIa receptor antagonist or an analogue or derivative thereof; or an endothelin receptor antagonist or an analogue or derivative thereof; or a peroxisome proliferators-activated receptor agonist or an analogue or derivative thereof; or an estrogen receptor agent or an analogue or derivative thereof; or somatostatin or an analogue or derivative thereof; or a JNK Kinase inhibitor or an analogue or derivative thereof; or a melanocortin analogue or derivative thereof; or a raf kinase inhibitor or analogue or derivative thereof; or a lysylhydroxylase inhibitor or an analogue or derivative thereof; or an IKK ½ inhibitor or an analogue or derivative thereof; or a cytokine modulator; or a cytokine antagonist; or the drug is water-insoluble.

The following are additional specific aspects of the present invention, which are exemplary only: in one aspect, the compositions and methods of the invention employ (i.e., include in a composition, or use in a method) a cell cycle inhibitor; in one aspect, the compositions and methods of the invention employ paclitaxel; in one aspect, the compositions and methods of the invention employ doxorubicin; in one aspect, the compositions and methods of the invention employ mitoxantrone; in one aspect, the compositions and methods of the invention employ podophyllotoxin (e.g., etoposide); in one aspect, the compositions and methods of the invention employ an immunomodulatory agents; in one aspect, the compositions and methods of the invention employ rapamycin; in one aspect, the compositions and methods of the invention employ everolimus; in one aspect, the compositions and methods of the invention employ tacrolimus; in one aspect, the compositions and methods of the invention employ biolimus; in one aspect, the compositions and methods of the invention employ a heat shock protein 90 antagonist; in one aspect, the compositions and methods of the invention employ geldanamycin; in one aspect, the compositions and methods of the invention employ a HMG CoA Reductase inhibitor; in one aspect, the compositions and methods of the invention employ simvastatin; in one aspect, the compositions and methods of the invention employ an IMPDH Inhibitor; in one aspect, the compositions and methods of the invention employ mycophenolic acid; in one aspect, the compositions and methods of the invention employ 1-alpha-25 dihydroxy vitamin D3; in one aspect, the compositions and methods of the invention employ an antimycotic agent; in one aspect, the compositions and methods of the invention employ sulconizole; in one aspect, the compositions and methods of the invention employ a P38 MAP kinase inhibitor; in one aspect, the compositions and methods of the invention employ SB220025.

In various aspects, the compositions of the present invention may be characterized by any one or more of the following criteria: the composition is in sterile form; the polymer contributes about 0.5-40 percent of the weight of the composition; the composition further comprises a solvent, e.g., water; the composition further comprises a buffer, e.g., a buffer that maintains the pH of the composition within the range of 4-10, or a buffer that maintains the pH of the composition within the range of 5-9, or a buffer that maintains the pH of the composition within the range of 6-8; or a buffer that maintains the pH of the composition at less than 6. Optionally, the buffer comprises phosphate.

In an optional embodiment, the compositions of the present invention, which may or may not include a drug, may include protein. In various aspects, which are exemplary only: the protein is collagen; the protein contains primary amino groups. Rather than contain protein, the compositions of the present invention may further compris polysaccharide, e.g., glysoaminoglycan.

Further details regarding the compositions of the present invention, and their method of manufacture, as described in further detail herein. In addition, and as also described in further detail herein, the present invention provides various methods of affecting biological processes in vivo. For example, in one aspect, the present invention provides a method of affecting biological processes in vivo comprising a) selecting an in vivo biological tissue comprising functional groups X; b) providing a composition comprising a synthetic polymer and a drug, the polymer comprising multiple activated groups Y, where Y is reactive with X; c) contacting the tissue of step a) with the composition of step b) under conditions where i) X reacts with Y and ii) biological processes in the vicinity of the tissue are affected by the drug. Optionally, the biological tissue has undergone surgical trauma prior to being contacted with the composition of step b), thereby placing the tissue at risk of adhesion formation. Adhesion formation is an undesired by-product of abdominal surgery, or the adhesion formation is an undesired by-product of cardiac surgery, or the adhesion formation is an undesired by-product of spinal surgery, or the adhesion formation is an undesired by-product of nasal surgery, or the adhesion formation is an undesired by-product of throat surgery, or the adhesion formation is an undesired by-product of breast implant.

In other optional embodiments of the methods for affecting biological processes in vivo, the biological tissue has undergone surgical trauma prior to being contacted with the composition of step b), the surgery being performed to excise tumor. Optionallly, the surgery is breast surgery; the surgery is breast tumor lumpectomy; the surgery is brain surgery; the surgery is hepatic resection surgery; the surgery is colon tumor resection surgery; or the surgery is neurosurgical tumor resection, where these types of surgery are exemplary only.

In one aspect, the present invention provides a method of reducing surgical adhesions comprising applying a multifunctional hydroxysuccinimidyl PEG derivative to a tissue surface. The multifunctional hydroxysuccinimidyl PEG derivative (e.g., tetra functional poly(ethylene glycol) succinimidyl glutarate) may be in the form of a solution, wherein the solution has a basic pH (e.g., pH of greater than 8). In one aspect, the multifunctional hydroxysuccinimidyl PEG derivative is not in admixture with any other tissue reactive compound. In another aspect, the multifunctional hydroxysuccinimidyl PEG derivative is not in admixture with any component that will react with the derivative. In one aspect, a method of reducing surgical adhesions is provided comprising applying a tissue reactive composition consisting essentially of a multifunctional hydroxysuccinimidyl PEG derivative to a tissue surface. In another aspect, a method of reducing surgical adhesions is provided comprising applying a tissue reactive composition consisting of a multifunctional hydroxysuccinimidyl PEG derivative to a tissue surface.

In various aspects of the invention, the tissue being contacted with the synthetic polymer having multiple activated groups is: the interior surface of a physiological lumen; a blood vessel; a Fallopian tube; or any tissue that has undergone balloon catheterization. These and other tissues that are advantageously contacted with a composition of the present invention are described in further detail herein.

These and related aspects of the present invention are described in greater detail by reference to the following Dawings and Detailed Description. Each publication cited above and herein is incorporated herein by reference in its entirety to describe and disclose the subject matter for which it is cited.

### BRIEF DESCRIPTION OF THE SEVERAL VIEWS OF THE DRAWING(S)

FIG. 1. Tetrafunctionally activated PEG succinimidyl glutarate (ester linkage) (SG-PEG).

FIG. 2. Tetrafunctionally activated propoxy succinimidyl PEG (ether linkage) (SP-PEG).

FIG. 3. Tetrafunctionally activated ethoxy succinimidyl PEG (ether linkage) (SE-PEG).

FIG. 4. Tetrafunctionally activated methoxy succinimidyl PEG (ether linkage) (SM-PEG).

FIG. 5. Tetrafunctionally activated succinamide succinimidyl PEG (amide linkage) (SSA-PEG).

FIG. 6. Tetrafunctionally activated carbonate succinimidyl PEG (ether linkage) (SC-PEG).

FIG. 7. Tetrafunctionally activated propion aldehyde PEG (A-PEG).

FIG. 8. Tetrafunctionally activated glycidyl ether PEG (E-PEG).

FIG. 9. Tetrafunctionally activated vinyl sulfone PEG (V-PEG).

FIG. 10. Tetrafunctionally activated Isocyanate PEG (1-PEG).

FIG. 11. Tetrafunctionally activated Maleimide PEG (Mal-PEG).

FIG. 12 is a plot of data showing the effect of 4-arm NHS PEG concentration on efficacy (percent adhesion) in the rat cecal sidewall surgical adhesions model.

FIG. 13 is a plot of data showing the effect of 4-arm NHS PEG concentration on efficacy (adhesion tenacity) in the rat cecal sidewall surgical adhesions model.

FIG. 14 is a plot of data showing the effect of buffer pH on the 4-arm NHS PEG efficacy (percent adhesion) in the rat cecal sidewall surgical adhesions model.

FIG. 15 is a plot of data showing the effect of buffer pH on the 4-arm NHS PEG efficacy (adhesion tenacity) in the rat cecal sidewall surgical adhesions model.

FIG. 16 is a schematic illustration showing sites of action within a biological pathway where Cell Cycle Inhitors may act to inhibit the cell cycle. The diagram shows locations where cell cycle inhibitors may exhibit their in vivo effect.

FIG. 17 is a graph showing % inhibition of human fibroblast cell proliferation as a function of Mitoxantrone concentration.

FIG. 18 is a graph showing % inhibition of nitric oxide production in RAW 264.7 cells as a function of Mitoxantrone concentration.

FIG. 19 is a graph showing % inhibition of TNFα production by THP-1 cells as a function of Bay 11-7082 concentration.

### DETAILED DESCRIPTION OF THE INVENTION

In accordance with the present invention, synthetic polymers that contain multiple activated groups can be used in various medical applications and medical device applications. More specifically, the present invention provides that a synthetic polymer containing multiple activated groups can be applied to a substrate that comprises functional groups that can react with the activated groups of the synthetic polymer. The substrate can be of biological or synthetic origin. Surfaces of biological origin include, but are not limited to, skin tissue, muscle tissue, vascular tissue occular tissue, epidermal tissue, epithelial tissue, adventitial tissue, abdominal tissue, brain tissue, nasal tissue, esophogeal tissue, lung tissue, spinal tissue, tendons and ligaments or any other class of tissue found in a mammal. Surfaces of synthetic origin include, but are not limited to, materials used to manufacture medical devices, materials used to coat medical devices, metals, plastics, ceramics, glass etc.

The present invention recognizes that a synthetic polymer containing one or more activated functional (electrophilic) groups (represented below as “Y”) will react with a surface containing one or more functional groups (nucleophilic groups; represented below as “X”) that are able to react with the activated functional groups of the synthetic polymer, resulting in the synthetic polymer being covalently bound to the surface, as follows:

Surface-Xₘ+polymer-Yₙ→polymer-Z-surface

wherein m≧1, n≧1, and m+n≧2;

X=—NH₂, —SH, —OH, —PH₂, —CO—NH—NH₂, etc., and can be the same or different;

Y=—CO₂N(COCH₂)₂, —CO₂H, —CHO, —CHOCH₂, —N═C═O,

—SO₂—CH═CH₂, —N(COCH₂)₂, —CO—O—CO—R, —S—S—(C₅H₄N), etc., and can be the same or different; and

Z=functional group resulting from the union of an activated functional group [electrophilic](Y) and the corresponding functional group [nucleophilic] (X) that is capable of reacting with the activated functional group.

As noted above, it is also contemplated by the present invention that X and Y may be the same or different, i.e., the polymer may have two different activated functional groups, and the surface may have two or more different functional groups that are capable of reacting with the activated functional groups of the polymer.

The backbone of each polymer preferably includes the polymerization residue of an alkylene oxide, particularly, ethylene oxide, propylene oxide, and mixtures thereof. Furthermore, the backbone of each polymer preferably includes a poly(alkylene oxide) moiety, e.g., the polymerization or copolymerization product of ethylene oxide, propylene oxide and the like.

Examples of difunctional alkylene oxides can be represented by:

Y-polymer-Y

wherein Y is as defined above, and the term “polymer” represents —(CH₂CH₂O)ₙ— or —(CH(CH₃)CH₂O)ₙ— or —(CH₂CH₂O)ₘ—(CH(CH₃)CH₂O)ₙ—.

Examples of Polymers

The required activated functional group Y is commonly coupled to the polymer backbone by a linking group (represented below as “Q”), many of which are known or possible.

Polymer-(Q-Y)ₙ

There are many ways to prepare the various functionalized polymers, some of which are listed below:

| wherein Q - | whole structure = |
|---|---|
| —O—(CH²)ₙ— | polymer —O—(CH₂)ₙ—Y |
| —S—(CH₂)ₙ— | polymer —S—(CH₂)ₙ—Y |
| —NH—(CH₂)ₙ— | polymer —NH—(CH₂)ₙ—Y |
| —O₂C—NH—(CH₂)ₙ— | polymer —O₂C—NH—(CH₂)ₙ—Y |
| —O₂C—(CH₂)ₙ— | polymer —O₂C—(CH₂)ₙ—Y |
| —O₂C—CR¹H— | polymer —O₂C—CRH—Y |
| —O—R²—CO—NH— | polymer —O—R—CO—NH—Y |

wherein
n = 1-12 in each case;
R¹ = H, CH₃, C₂H₅, etc.;
R₂ = CH₂, CO—NH—CH₂CH₂.

For example, when Q=OCH₂CH₂; Y=—CO₂N(COCH₂)₂; and X=—NH₂, —SH, or —OH, the resulting reactions and Z groups would be as follows:

surface-NH₂+polymer-OCH₂CH₂CO₂—N(COCH₂)₂→Polymer-OCH₂CH₂CO—NH-surface (amide)

surface-SH+polymer-OCH₂CH₂CO₂—N(COCH₂)₂→Polymer-OCH₂CH₂CO—S-surface (thioester)

surface-OH+polymer-OCH₂CH₂CO₂—N(COCH₂)₂ Polymer-OCH₂CH₂CO—O-surface (ester)

An additional group, represented below as “D”, can be inserted between the polymer and the linking group to alter the degradation profile and release of the surface attached polymer.

surface-X+polymer-D-Q-Y→surface-Z-Q-D-polymer

Some useful biodegradable groups “D” include lactide, glycolide, ε-caprolactone, poly(.alpha.-hydroxy acid), poly(amino acids), poly(anhydride), poly(orthoesters), polyesters comprising residues from one or more monomers selected from lactide, lactic acid, glycolide, glycolic acid, ε-caprolactone, trimethylene carbonate, 1,4-dioxane-2-one, and 1,5-dioxepan-2one, peptides, carbohydrates and various di- or tripeptides.

In another preferred embodiment, the compounds each have 12 functional groups. Such compounds are formed from reacting a first tetrafunctionally activated polymer with a four tetrafunctionally activated polymers, wherein the functional groups of each of the two compounds are a reaction pair, to form “12-arm” functionally activated polymers. An example of a such a “12-arm” compound is dodeca-sulfhydryl-PEG, 50,000 mol. wt., which is constructed from a core tetra-functional succinimide ester PEG coupled to four (exterior) tetra-functional sulfhydryl-PEG molecules. Such polymers range in size from over 10,000 mol. wt. to greater than 100,000 mol. wt. depending on the molecular weight of the tetra-functionally activated polymer starting materials.

Other types of multifunctional polymers can easily be synthesized using routine synthesis. However, care should be taken to produce multi-arm products with consistent arm lengths to avoid steric hindrance of the reactive groups. Accordingly, activated polymers that are suitable for use in the present invention may have a variety of geometric shapes and configurations. Exemplary polymers according to the present invention, as well as methods of their manufacture and use, are described in U.S. Pat. Nos. 5,874,500; 6,051,648; 6,166,130; 6,312,725; 6,323,278; and 6,458,889.

Compound Core

As described above, each of the compounds has multiple activated functional groups, either succinimidyl groups or maleimide reactive groups. The non-reactive remainder of the compound is considered to be its “core”.

The polymer core may be a synthetic polyamino acid, a polysaccharide, or a synthetic polymer. A preferred polymer core material is a synthetic hydrophilic polymer. Suitable synthetic hydrophilic polymers include, inter alia, polyalkylene oxide, such as polyethylene oxide ((CH₂CH₂O)ₙ), polypropylene oxide ((CH(CH₃)CH₂O)ₙ) or a polyethylene/polypropylene oxide mixture ((CH₂CH₂O)ₙ—CH(CH₃)CH₂O)ₙ). A particularly preferred synthetic hydrophilic polymer is a polyethylene glycol (PEG) having a molecular weight (number average or weight average) within the range of about 100 to about 100,000 mol. wt., more preferably about 1,000 to about 20,000 mol. wt. More preferably still, when the polymer core is polyethylene glycol, it generally has a molecular weight within the range of about 7,500 to about 20,000 mol. wt. Most preferably, the polyethylene glycol has a molecular weight of approximately 10,000 mol. wt.

Polyalkylene oxides that have multiple activated functional groups are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); The PEG Shop online catalogue; and Shearwater Polymers, Inc. Catalog, Polyethylene Glycol Derivatives, Huntsville, Ala. (2000-2001).

As described in further detail herein, a compound having multiple activatable groups can be applied to tissue, whereupon the compound will react and form covalent bonds with reactive functional groups of the tissue. In a preferred embodiment, the compound having multiple activatable groups is the only tissue-reactive compound being added to the tissue, and furthermore, the compound is not combined with or otherwise reacted with any other compound, i.e., it reacts only with the tissue and/or the proteins associated with the tissue. Thus, in a preferred embodiment, tissue is reacted with a compound having multiple activatable groups, and neither that tissue nor that compound is reacted with any other chemical. These compounds having multiple activated groups, upon reaction with tissue, impart desirable properties to the tissue, and are particularly useful in instances where reduced adhesion of the tissue to other tissue is desired.

In another aspect, the compounds are reacted with tissue in instances where restenosis is a concern. Restenosis refers to a re-narrowing or blockage of an artery at the same site where treatment, such as an angioplasty or stent procedure, has already taken place. The end result of restenosis is a narrowing in the artery caused by a build-up of substances that may eventually block the flow of blood. The adhesion of a compound having multiple activatable groups to tissue where restenosis is a concern may be used to mitigate the build-up of undesirable substances at the tissue site.

In another aspect, the compounds are reacted with tissue in instances where enhanced lubricity is desired. In other words, the compounds are useful in instances where it is desired that the treated tissue adhere less readily to other tissue. In a related aspect, the compounds are reacted with the surface of a medical device, thereby imparting increased lubricity to the device. Again, in a preferred aspect, the surface (either tissue surface or device surface) is reacted with a compound having multiple activatable groups, and neither that surface nor that compound is reacted with any other chemical.

For use in a composition for the prevention of surgical adhesions, or to address concerns of restenosis, or wherever enhanced lubricity on the surface of tissue or a medical device is desired, a preferred activated polymer is as follows: the activated functional group-containing compound is the tetrafunctional PEG, pentaerythritol poly(ethylene glycol) ether tetra-succinimidyl glutarate (10,000 mol. wt.). This “four-arm” PEGs is formed by ethoxylation of pentaerythritol, where each of the four chains is approximately 2,500 mol. wt., and then derivatized to introduce the functional groups onto each of the four arms. Also preferred are analogous poly(ethylene glycol)-like compounds polymerized from di-glycerol instead of pentaerythritol.

Multifunctionally active small organic molecule can also be use in these applications. Such compounds include the di-functional di-succinimidyl esters and di-maleimidyl compounds, as well as other well known commercially available compounds (Pierce Chemical Co., Rockford, Ill.). In addition, one of skill in the art could easily synthesize a low molecular weight multi-functional reactive compound using routine organic chemistry techniques. On such compound is a penta-erythritol coupled to four glutarates, with each arm capped with N-hydroxy-succinimidyl esters (NHS). Analogous compounds can be synthesized from inositol (radiating 6 arm), lactitol (9 arm) or sorbitol (linear 6-arm). The end-capped reactive group can just as easily be maleimidyl, vinyl-sulfone, etc., instead of NHS.

Reactive Groups and Matrix Linkages

In the present invention, the most preferable linkage, Z, comprises a covalent bond between a sulfur, oxygen or nitrogen atom in the surface compound and the carbon or sulfur atom in the activated functional group containing compound. Accordingly, the linkage may be an amide, a thioester, a thioether, a disulfide, or the like. A wide variety of sulfhydryl-reactive groups and the types of linkages they form when reacted with sulfhydryl groups are well known in the scientific literature. For example, see Bodanszky, M., Principles of Peptide Synthesis, 2nd ed., pages 21 to 37, Springer-Verlog, Berlin (1993); and Lundbland, R. L., Chemical Reagents for Protein Modification, 2nd ed., Chapter 6, CRC Press, Boca Raton, Fla. (1991).

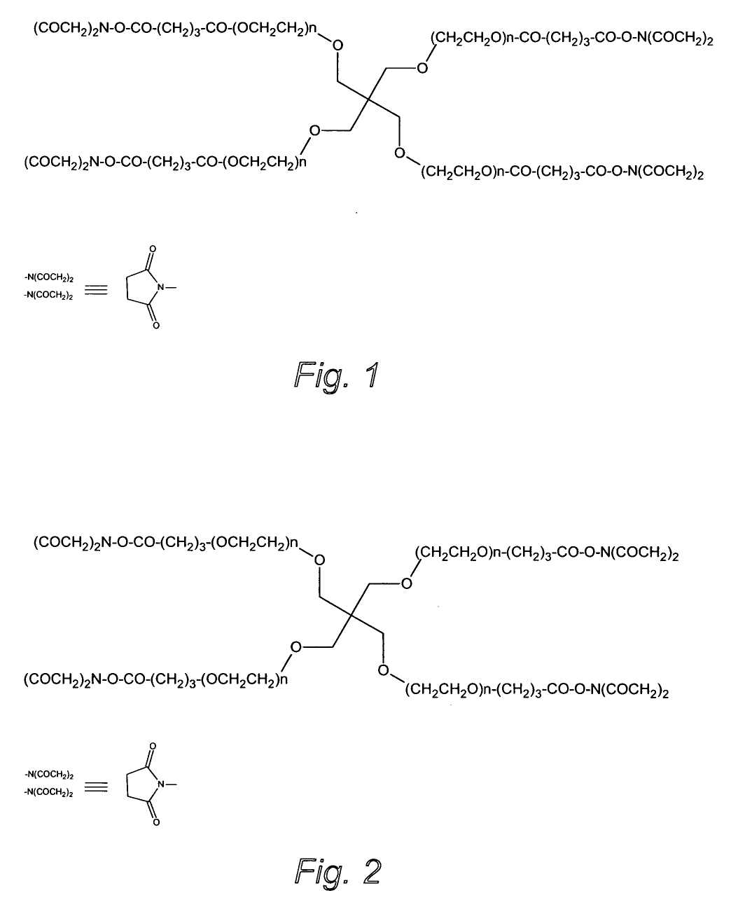

For most applications, activated functional groups that react with sulfhydryl groups to form thioester linkages or amine groups to form amides are preferred. Such compounds are depicted in FIG. 1 and include, inter alia, the following compounds, with the numbers in parentheses corresponding to the structures shown in FIG. 1: mixed anhydrides, such as PEG-glutaryl-acetyl-anhydride (1), PEG-glutaryl-isovaleryl-anhydride (2), PEG-glutaryl-pivalyl-anhydride (3) and related compounds as presented in Bodanszky, p. 23; Ester derivatives of phosphorus, such as structures (4) and (5); ester derivatives of p-nitrophenol (6) of p-nitrothiophenol (7), of pentafluorophenol (8), of structure (9) and related active esters as presented by Bodanszky, pp. 31-32, and Table 2; esters of substituted hydroxylamines, such as those of N-hydroxy-phthalimide (10), N-hydroxy-succinimide (11), and N-hydroxy-glutarimide (12), as well as related structures in Bodanszky; Table 3; esters of 1-hydroxybenzotriazole (13), 3-hydroxy-3,4-dihydro-benzotriazine-4-one (14) and 3-hydroxy-3,4-dihydro-quinazoline-4-one; derivatives of carbonylimidazole; and isocyanates. With these compounds, auxiliary reagents can also be used to facilitate bond formation, such as 1-ethyl-3-(3-dimethylaminopropyl]carbodiimide can be used to facilitate coupling of carboxyl groups (i.e., glutarate and succinate) with sulfhydryl groups.

In addition to the sulfhydryl reactive compounds that form thioester linkages, various other compounds can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups, such as ortho pyridyl disulfide, 3-nitro-2-pyridenesulfenyl, 2-nitro-5-thiocyanobenzoic acid, 5,5′-dithio-bis(2-nitrobenzoic acid), derivatives of methane-thiosulfate, and 2,4-dinitrophenyl cysteinyl disulfides. In such instances, auxiliary reagents, such as the hydrogen peroxide or di-tert-butyl ester of azodicarboxylic acid, can be used to facilitiate disulfide bond formation.

Yet another class of sulfhydryl reactive groups form thioether bonds with sulfhydryl groups. Such groups include, inter alia, iodoacetamide, N-ethylmaleimide and other maleimides, including dextran maleimides, mono-bromo-bimane and related compounds, vinylsulfones, epoxides, derivatives of O-methyl-isourea, ethyleneimines, aziridines, and 4-(aminosulfonyl-)₇-fluoro-2,1,3-benzoxadiazole.

Chain Extenders

Functional groups may be directly attached to the compound core, or they may be indirectly attached through a chain extender. Such chain extenders are well known in the art. See, for example, PCT WO 97/22371, which describes “linking groups” that would be suitable for use as chain extenders in the compositions of the present invention. Chain extenders are useful to avoid stearic hindrance problems that are sometimes associated with the formation of direct linkages between molecules. Alternatively, chain extenders may be used to link several multifunctionally activated compounds together to make larger molecules. In a particularly preferred embodiment, the chain extender can also be used to alter the degradative properties of the compositions after administration and resultant gel formation. For example, chain extenders can be incorporated into the activated polymers to promote hydrolysis, to discourage hydrolysis, or to provide a site for enzymatic degradation. Chain extenders can also activate or suppress activity of the amine reactive or sulfhydryl-reactive groups. For example, bulky nearby groups for the activated functional groups are anticipated to diminish coupling rates, due to steric hindrance. Electron-withdrawing groups adjacent to the reactive carbonyl of glutaryl-N-hydroxysuccinimidyl would be anticipated to make this carbonyl carbon even more reactive with a surface amino or sulfhydryl group partner.

Chain extenders may provide sites for degradation, i.e., hydrolysable sites. Examples of hydrolysable chain extenders include, inter alia, alpha-hydroxy acids such as lactic acid and glycolic acid; poly(lactones) such as caprolactone, valerolactone, gamma butyl lactone and p-dioxanone; poly(amino acids); poly(anhydrides) such as glutarate and succinate; poly(orthoesters); poly(orthocarbonates) such as trimethylene carbonate; and poly(phosphoesters). Examples of non-degradable chain extenders include, inter alia, succinimide, propionic acid and carboxymethylate. See, for example, PCT WO 99/07417. Examples of enzymatically degradable chain extenders include Leu-Gly-Pro-Ala, which is degraded by collagenase; and Gly-Pro-Lys, which is degraded by plasmin.

Synthetic Polymers

In order to prepare the compositions of the present invention, it is first necessary to provide a first synthetic polymer containing two or more activated functional groups, such as succinimidyl groups or malemide groups. As used herein, the term “polymer” refers inter alia to polypolyalkyls, polyamino acids and polysaccharides. Additionally, for device, implant, external or oral use, the polymer may be polyacrylic acid or carbopol.

As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing electrophilic groups are also referred to herein as “multifunctionally activated synthetic polymers”. The term “multifunctionally activated” (or, simply, “activated”) refers to synthetic polymers which have, or have been chemically modified to have, two or more electrophilic groups which are capable of reacting with nucleophilic groups to form covalent bonds. Types of multifunctionally activated synthetic polymers include difunctionally activated, tetrafunctionally activated, and star-branched polymers.

Multifunctionally activated synthetic polymers for use in the present invention must contain at least two, more preferably, at least three, functional groups

Synthetic Polymers Containing Multiple Activated Functional Groups

Synthetic polymers containing multiple activated functional groups are also referred to herein as “activated polymers.” For use in the present invention, the activated multifunctionally synthetic polymers must contain at least two, more preferably, at least three, activated functional groups and most preferably, at least four activated functional groups.

Preferred activated polymers for use in the compositions of the invention are polymers which contain two or more succinimidyl groups capable of forming covalent bonds with electrophilic groups on other molecules. Succinimidyl groups are highly reactive with materials containing primary amino (—NH₂) groups, such as tissue surfaces, poly(lysine), amino functionalized polymers or collagen. Succinimidyl groups are slightly less reactive with materials containing thiol (—SH) groups, such as multi-thiol PEG, tissue surfaces, thiol functionalized polymers or synthetic polypeptides containing multiple cysteine residues.

As used herein, the term “containing two or more succinimidyl groups” is meant to encompass polymers that are commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

Hydrophilic Polymers

Hydrophilic polymers and, in particular, various polyethylene glycols, are preferred for use in the compositions of the present invention. As used herein, the term “PEG” refers to polymers having the repeating structure (OCH₂CH₂)ₙ.

Structures for some specific, tetrafunctionally activated forms of PEG are shown in FIGS. 1 to 11. As depicted in the figures, the succinimidyl group is a five-member ring structure represented as —N(COCH₂)₂.

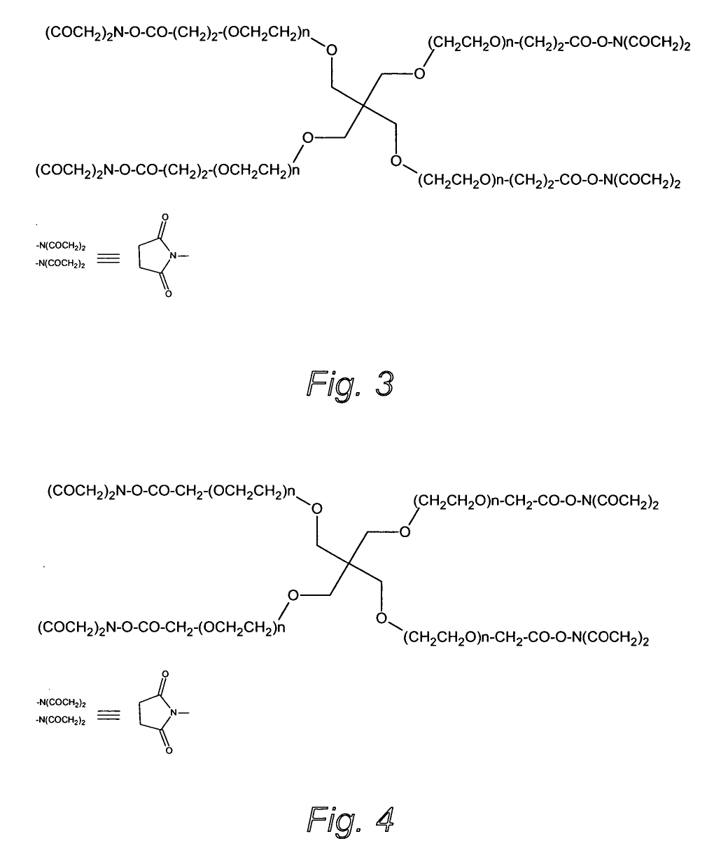

FIG. 1 shows the structure of tetrafunctionally activated PEG succinimidyl glutarate, referred to herein as SG-PEG. Another activated form of PEG is referred to as PEG succinimidyl propionate (SE-PEG). The structural formula for tetrafunctionally activated SE-PEG is shown in FIG. 2. In a general structural formula for the compound, the subscript 3 is replaced with an “m”. In the embodiment shown in FIG. 4, m=3, in that there are three repeating CH₂ groups on either side of the PEG.

The structure in FIG. 2 results in a conjugate which includes an “ether” linkage which is less subject to hydrolysis. This is distinct from the conjugate shown in FIG. 1, wherein an ester linkage is provided. The ester linkage is subject to hydrolysis under physiological conditions.

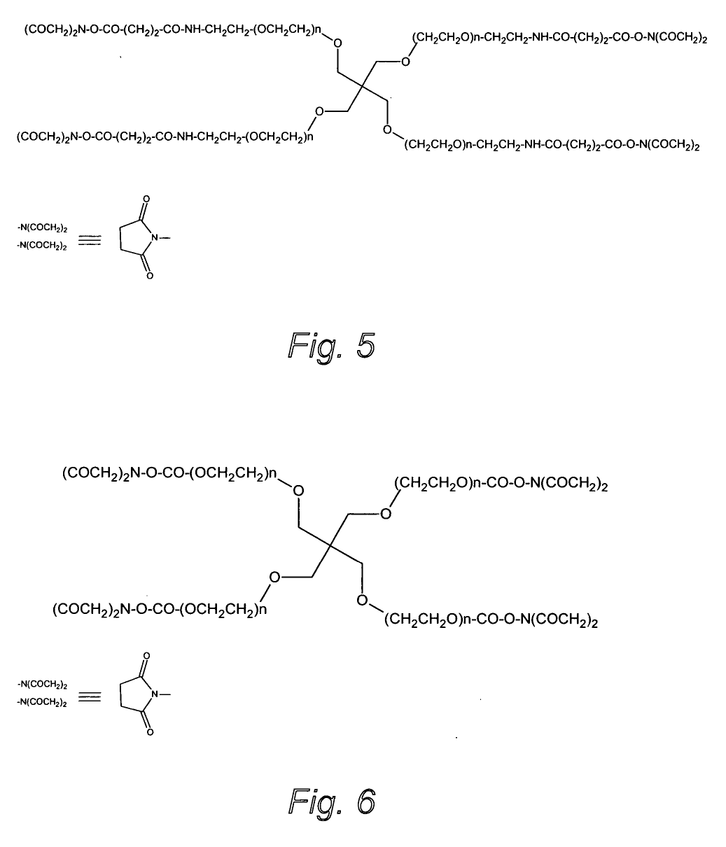

Yet another functionally activated form of polyethylene glycol is shown in FIG. 3.

Another functionally activated PEG similar to the compounds of FIGS. 2 and 3 is provided in FIG. 4.

Another functionally activated form of PEG is referred to as PEG succinimidyl succinamide (SSA-PEG is shown in FIG. 5. In the structure shown in FIG. 5, m=2; however, related compounds, wherein m=1 or m=3-10, may also be used in the compositions of the invention.

The structure in FIG. 5 results in a conjugate which includes an “amide” linkage which, like the ether linkage previously described, is less subject to hydrolysis and is therefore more stable than an ester linkage.

Yet another activated form of PEG is provided when m=0. This compound is referred to as PEG succinimidyl carbonate (SC-PEG). The structural formula of tetrafunctionally activated SC-PEG is shown in FIG. 6.

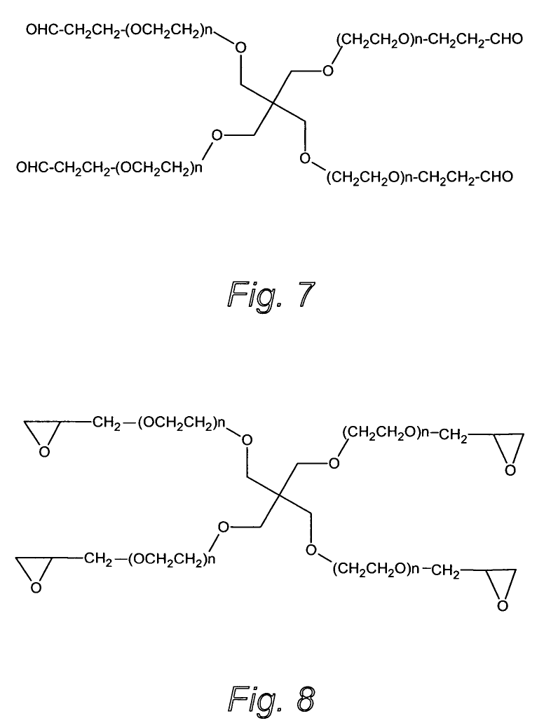

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propion aldehyde (A-PEG), the tetrafunctionally activated form of which is shown in FIG. 7. The linkage shown in FIG. 5 is referred to as a —(CH₂)ₘ—NH-linkage, where m=1-10.

Yet another form of activated polyethylene glycol is functionally activated PEG glycidyl ether (E-PEG), of which the tetrafunctionally activated compound is shown in FIG. 8.

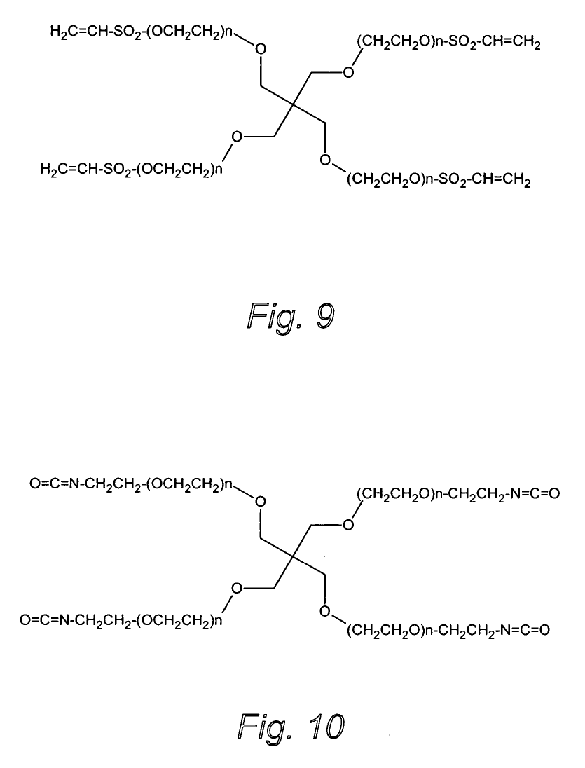

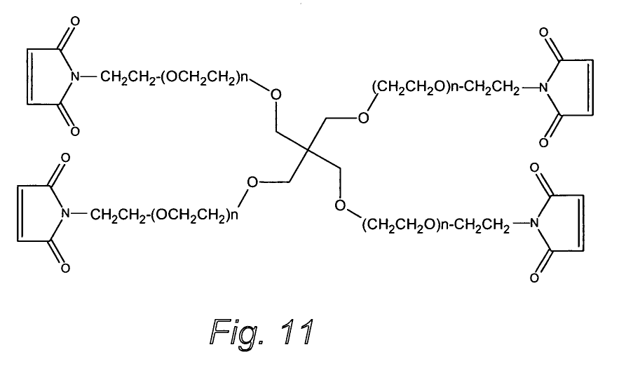

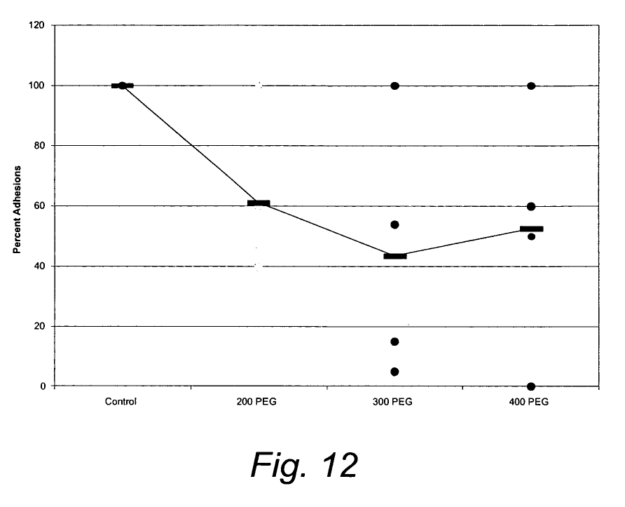

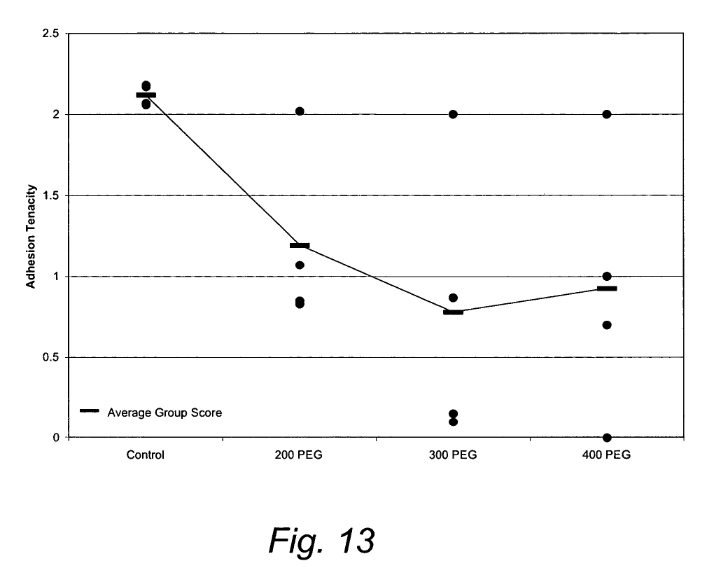

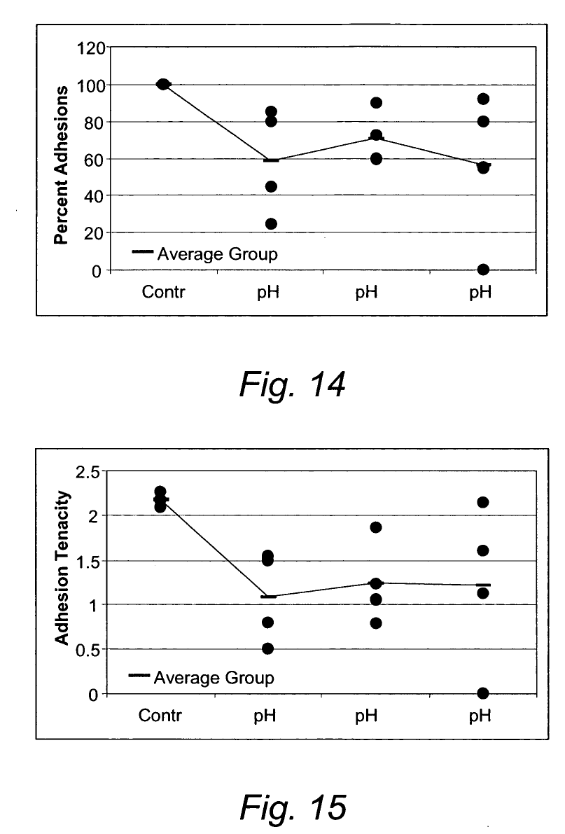

Another activated derivative of polyethylene glycol is functionally activated PEG-vinylsulfone (V-PEG), which is shown in FIG. 9. Another activated derivative of polyethylene glycol is functionally activated PEG-isocyanate (1-PEG), which is shown in FIG. 10. Another activated polyethylene glycol is functionally activated vinyl sulfone PEG, which is shown in FIG. 11.

Preferred multifunctionally activated polyethylene glycols for use in the compositions of the present invention are polyethylene glycols containing succinimidyl groups, such as SG-PEG and SE-PEG (shown in FIGS. 1-4), preferably in trifunctionally or tetrafunctionally activated form.

Many of the activated forms of polyethylene glycol described above are now available commercially from SunBio PEG-SHOP, Anyang City, South Korea, Shearwater Polymers, Huntsville, Ala., and Union Carbide, South Charleston, W.Va.

Hydrophobic Polymers

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers that contain a relatively small proportion of oxygen or nitrogen atoms.

Hydrophobic polymers which already contain two or more succinimidyl groups include, without limitation, disuccinimidyl suberate (DSS), bis(sulfosuccinimidyl) suberate (BS³), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogues and derivatives. The above-referenced polymers are commercially available from Pierce (Rockford, Ill.), under Catalogue #s.21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Derivatization of Polymers to Contain Functional Groups

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.).

According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—CH₂)₆—COOH), and hexadecanedioic acid (HOOC—CH₂)₁₄—COOH) are derivatized by the addition of succinimidyl groups to produce difunctionally activated polymers.

Polyamines such as ethylenediamine (H₂N—CH₂CH₂—NH₂), tetramethylenediamine (H₂N—(CH₂)₄—NH₂), pentamethylenediamine (cadaverine) (H₂N—(CH₂)₅—NH₂), hexamethylenediamine (H₂N—(CH₂)₆—NH₂), bis(2-hydroxyethyl)amine (HN—(CH₂ CH₂ OH)₂), bis(2)aminoethyl)amine (HN—(CH₂CH₂NH₂)₂), and tris(2-aminoethyl)amine (N—(CH₂CH₂NH₂)₃) can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

Preparation of Compositions

In general, the concentrations of the activated polymer used to prepare the compositions of the present invention will vary depending upon a number of factors, including the types and molecular weights of the particular synthetic polymers used and the desired end use application.

In general, we have found that when using multi-succinimidyl PEG as the synthetic polymer, it is preferably used at a concentration in the range of about 0.5 to about 40 percent by weight of the final composition. For example, a final composition having a total weight of 1 gram (1000 milligrams) would contain between about 5 to about 400 milligrams of multi succinimidyl PEG.

Because polymers containing multiple activated functional groups also have the potential to react with water, the activated polymer is generally prepared, packaged and stored in a dry form to prevent the loss of activity of the activated functional groups due to reaction with water which typically occurs upon exposure of such activated groups to aqueous media. Processes for preparing synthetic hydrophilic polymers containing multiple electrophylic groups in sterile, dry form are set forth U.S. application Ser. No. 08/497,573, filed Jun. 30, 1995. For example, the dry synthetic polymer may be compression molded into a thin sheet or membrane, which can then be sterilized using gamma or, e-beam irradiation. The resulting dry membrane or sheet can be cut to the desired size or chopped into smaller size particulates.

Incorporation of Other Components into the Activated Synthetic Polymer

Naturally occurring proteins, such as collagen, and derivatives of various naturally occurring polysaccharides, such as glycosaminoglycans, can additionally be incorporated into the compositions of the invention. When these other components also contain functional groups that will react with the functional groups on the synthetic polymers, their presence during mixing and/or crosslinking of the first and second synthetic polymer will result in formation of a crosslinked synthetic polymer-naturally occurring polymer matrix. In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix.

In general, glycosaminoglycans must be chemically derivatized by deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

Similarly, electrophilic groups on the second synthetic polymer will react with primary amino groups on lysine residues or thiol groups on cysteine residues of certain naturally occurring proteins. Lysine-rich proteins such as collagen and its derivatives are especially reactive with electrophilic groups on synthetic polymers. As used herein, the term “collagen” is intended to encompass collagen of any type, from any source, including, but not limited to, collagen extracted from tissue or produced recombinantly, collagen analogues, collagen derivatives, modified collagens, and denatured collagens such as gelatin. Covalent binding of collagen to synthetic hydrophilic polymers is described in detail in commonly assigned U.S. Pat. No. 5,162,430, issued Nov. 10, 1992, to Rhee et al.

In general, collagen from any source may be used in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022, issued Jun. 27, 1995, to Palefsky et al., discloses methods of extracting and purifying collagen from the human placenta. U.S. application Ser. No. 08/183,648, filed Jan. 18, 1994, discloses methods of producing recombinant human collagen in the milk of transgenic animals, including transgenic cows. The term “collagen” or “collagen material” as used herein refers to all forms of collagen, including those which have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Aesthetics at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its tacky consistency, nonfibrillar collagen is generally preferred for use in compositions of the invention that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Because it is opaque and less tacky than nonfibillar collagen, fibrillar collagen is less preferred for use in bioadhesive compositions. However, as disclosed in U.S. application Ser. No. 08/476,825, fibrillar collagen, or mixtures of nonfibrillar and fibrillar collagen, may be preferred for use in adhesive compositions intended for long-term persistence in vivo, if optical clarity is not a requirement.

For compositions intended for use in tissue augmentation, fibrillar collagen is preferred because it tends to form stronger crosslinked gels having greater long-term persistency in vivo than those prepared using nonfibrillar collagen.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen.

For use in tissue adhesion as discussed below, it may also be desirable to incorporate proteins such as albumin, fibrin or fibrinogen into the crosslinked polymer composition to promote cellular adhesion.

In addition, the introduction of hydrocolloids such as carboxymethylcellulose may promote tissue adhesion and/or swellability.

Administration of the Synthetic Polymer Compositions

The compositions of the present invention may be administered in a number of different ways.

In one embodiment, the activated polymer can be applied to the desired surface as a solid. The preferred solid is in the form of a powder. The activated polymer may be applied to the surface by sprinkling, brushing or spraying the powder onto the surface. In the case where the surface is tissue, then the solid powder form of the activated polymer will slowly hydrate. This will then allow the activated functional groups to react with the appropriate surface functional groups. For the succinimidyl activated groups, it is anticipated that this reaction will be relatively slow since the pH of the adsorbed fluid is anticipated to be in the pH range of about 7.2-7.4.

In another embodiment, the activated polymer can be applied to the surface in the presence of a second solid compound. The second compound is one that, upon dissolution following absorption of fluid, will create a basic environment (e.g., pH>about 7.5). This second solid compound can be applied prior to, at the same time as or after the application activated polymer. When the activated polymer comprises succinimidyl groups, the creation of a basic environment will increase the reaction rate of the activated polymer with the suface to which it was applied.

In another embodiment, the solid activated powder can be dissolved in a biologically acceptable solution. In the preferred embodiment, this solution is a buffered aqueous solution that has a pH of less than about 6.5.

The buffering capacity of the aqueous solution can be altered depending on pH requirements of the specific application. This solution can then be applied to the desired surface by brushing, dropping or spraying the solution onto the tissue.

In another embodiment, a second biologically acceptable solution can be applied prior to, at the same time of or after the application of the activated polymer solution (prepared as described above). In the preferred embodiment, the second biologically acceptable solution is a buffered aqueous solution with a pH greater than about 7.6.

In another embodiment, the activated polymer can be applied in the solid form (as described above) with a second biologically acceptable solution being applied prior to, at the same time of or after the application of the activated polymer in the solid form. In the preferred embodiment, the second biologically acceptable solution is a buffered aqueous solution with a pH greater than about 7.6.

In another embodiment, the compositions of this invention can further comprise a viscosity modifying agent. In the preferred embodiment, the viscocity modifying agent will increase the solution viscosity of the composition. Examples of viscosity modifying agents include, but are not limited to hyaluronic acid, polyalkylene oxides (e.g., PLURONIC F127 from BASF Corporation, Mount Olive, N.J.), glycerol, carboxymethyl cellulose, sodium alginate, chitosan, dextran, dextran sulfate and collagen. These viscosity modifying agents can be chemically modified to prevent reation with the activated polymers. Other visocity modifying agents known in the art can also be incorporated into the compositions of this invention.

As described above, the compositions of this invention can be applied directly, by brushing on to the surface, by dipping the surface into the composition or by spraying the composition onto the surface. U.S. Pat. Nos. 6,152,943, 6,15,201, and 6,328,229 and U.S. Publication No. 2002/0082636 describe different devices that can be used to apply the compositions of this invention and are hereby incorporated by reference.

Use of Activated Synthetic Polymers to Deliver Biologically Active Agents

The polymer compositions of the present invention may also be used for localized delivery of various drugs and other biologically active agents. The term “biologically active agent” or “active agent” as used herein refers to organic molecules which exert biological effects in vivo. Briefly stated, in one aspect the present invention provides compositions and methods for the treatment of surgical adhesions. In another aspect, the present invention provides compositions and methods for mitigating restenosis. In another aspect, the present invention provides compositions and methods for inhibiting fibrosis. In another aspect, the present invention provides compositions and methods for enhancing the lubricity of a surface, where in one embodiment that surface is tissue, while in another embodiment that surface is a surface of a medical device.

One aspect of the invention involves pharmacological alteration of cellular and/or non-cellular processes involved in the development and/or maintenance of surgical adhesions and/or restenosis and/or inhibition of one or more processes involved in fibrosis. Thus, pharmacological agents within the scope of this invention include but are not limited to those which inhibit one or a combination of processes such as cell division, cell secretion, cell migration, cell adhesion, cytokine (e.g., TNF alpha, IL-1, IL-6), (or other inflammatory activator e.g. chemokines (e.g., MCP-1, IL-8)) production and/or release, immunomodulation, angiogenesis, and/or free radical formation and/or release.

Suitable fibrosis, adhesions or stenosis-inhibiting agents may be readily determined based upon the in vitro and in vivo (animal) models such as those provided in Examples 8-13. Numerous fibrosis, adhesion and/or stenosis-inhibiting therapeutic compounds have been identified that are of utility in the invention including:

1. Angiogenesis Inhibitors

In one embodiment, the pharmacologically active compound is an angiogenesis inhibitor (e.g., 2-ME (NSC-659853), PI-88 (D-Mannose, 0-6-O-phosphono-Alpha-D-mannopyranosyl-(1-3)—O-Alpha-D-mannopyranosyl-(1-3)—O-Alpha-D-mannopyranosyl-(1-3)—O-Alpha-D-mannopyranosyl-(1-2)-hydrogen sulphate [CAS]), thalidomide (1H-Isoindole-1,3(2H)-dione, 2-(2,6-dioxo-3-piperidinyl)-[CAS]), CDC-394, CC-5079, ENMD-0995 (S-3-amino-phthalidoglutarimide), AVE-8062a, Vatalanib, SH-268, Halofuginone hydrobromide)) or an analogue or derivative thereof.

2. 5-Lipoxygenase Inhibitors & Antagonists

In another embodiment, the pharmacologically active compound is a 5-lipoxygenase inhibitor or antagonist (e.g., licofelone (ML3000), 2-uredo thiophene/2 amino thiophene, 15-deoxy-Prostaglandin J2, Wy-50295 (2-Naphthaleneacetic acid, Alpha-methyl-6-(2-quinolinylmethoxy)-, (S)-[CAS]), ONO-LP-269 (2,11,14-Eicosatrienamide, N-(4-hydroxy-2-(1H-tetrazol-5-yl)-8-quinolinyl]-, (E,Z,Z)-[CAS]), licofelone (1H-Pyrrolizine-5-acetic acid, 6-(4-chlorophenyl)-2,3-dihydro-2,2-dimethyl-7-phenyl-[CAS]), CMI-568 (Urea, N-butyl-N-hydroxy-N′-(4-(3-(methylsulfonyl)-2-propoxy-5-(tetrahydro-5-(3,4,5-trimethoxyphenyl)-2-furanyl]phenoxy]butyl]-,trans-[CAS]), IP-751 ((3R,4R)-(delta6)-THC-DMH-11-oic acid), PF-5901 (Benzenemethanol, Alpha-pentyl-3-(2-quinolinylmethoxy)-[CAS]), LY-293111 (Benzoic acid, 2-(3-(3-((5-ethyl-4′-fluoro-2-hydroxy(1,1′-biphenyl]-4-yl)oxy]propoxy]-2-propylphenoxy]-[CAS]), RG-5901-A (Benzenemethanol, Alpha-pentyl-3-(2-quinolinylmethoxy)-, hydrochloride [CAS]), rilopirox (2(1H)-Pyridinone, 6-((4-(4-chlorophenoxy)phenoxy]methyl]-1-hydroxy-4-methyl-[CAS]), L-674636 (Acetic acid, ((4-(4-chlorophenyl)-1-(4-(2-quinolinylmethoxy)phenyl)butyl)thio)-AS]), 7-((3-(4-methoxy-tetrahydro-2H-pyran-4-yl)phenyl]methoxy]-4-phenylnaphtho(2,3-c]furan-1 (3H)-one, MK-886 (1H-Indole-2-propanoic acid, 1-((4-chlorophenyl)methyl]-3-((1,1-dimethylethyl)thio]-Alpha,Alpha-dimethyl-5-(1-methylethyl)-[CAS]), quiflapon (1H-Indole-2-propanoic acid, 1-((4-chlorophenyl)methyl]-3-((1,1-dimethylethyl)thio]-Alpha,Alpha-dimethyl-5-(2-quinolinylmethoxy)-[CAS]), quiflapon (1H-Indole-2-propanoic acid, 1-((4-chlorophenyl)methyl]-3-((1,1-dimethylethyl)thio]-Alpha,Alpha-dimethyl-5-(2-quinolinylmethoxy)-[CAS]), docebenone (2,5-Cyclohexadiene-1,4-dione, 2-(12-hydroxy-5,10-dodecadiynyl)-3,5,6-trimethyl-[CAS]), zileuton (Urea, N-(1-benzo(b]thien-2-ylethyl)-N-hydroxy-[CAS])) or an analogue or derivative thereof.

3. Chemokine Receptor Antagonists CCR (1, 3, & 5)

In another embodiment, the pharmacologically active compound is a chemokine receptor antagonist (e.g., AMD-3100 (Anormed), ONO-4128 (1,4,9-Triazaspiro(5.5)undecane-2,5-d ione,1-butyl-3-(cyclohexylmethyl)-9-((2,3-dihydro-1,4-benzodioxin-6-yl)methyl-[CAS]), L-381, CT-112 (L-Arginine, L-threonyl-L-threonyl-L-seryl-L-glutaminyl-L-valyl-L-arginyl-L-prolyl-[CAS]), AS-900004, SCH—C, ZK-811752, PD-172084, UK-427857, SB-380732, vMIP 11, SB-265610, DPC-168, TAK-779 (N,N-Dimethyl-N-(4-(2-(4-methylphenyl)-6,7-dihydro-5H-benzocyclohepten-8-ylcarboxamido]benyl]tetrahydro-2H-pyran-4-aminium chloride), TAK-220, KRH-1120) or an analogue or derivative thereof.

4. Cell Cycle Inhibitors

In another embodiment, the pharmacologically active compound is a cell cycle inhibitor or an analogue or derivative thereof. In related embodiments, the cell-cycle inhibitor is a taxane (e.g., paclitaxel, or an analogue or derivative thereof), an antimetabolite, an alkylating agent, or, a vinca alkaloid. In another embodiment, the cell-cycle inhibitor is camptothecin, or an analogue or derivative thereof. Other suitable compounds include mitoxantrone, etoposide, 5-fluorouracil, doxorubicin, methotrexate, paclitaxel, Peloruside A—a microtubule stabilizing agent, Mitomycin-C, and CDK-2 inhibitors.

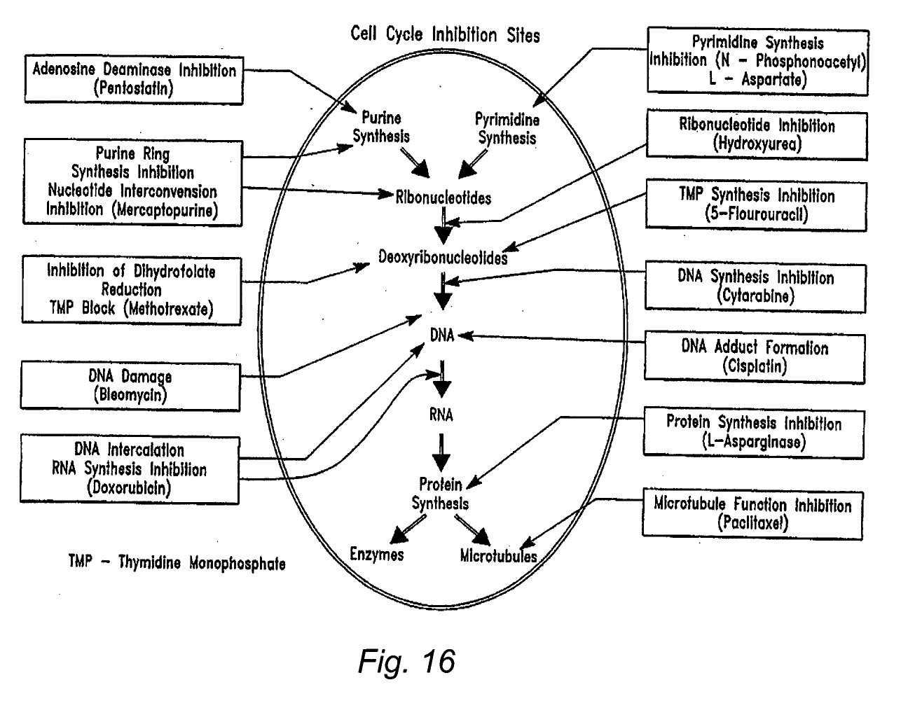

“Cell Cycle Inhibitor” as used herein refers to any protein, peptide, chemical or other molecule which delays or impairs a dividing cell's ability to progress through the cell cycle and replicate. A wide variety of methods may be utilized to determine the ability of a compound to inhibit the cell cycle including univariate analysis of cellular DNA content and multiparameter analysis (see the Examples). A Cell Cycle Inhibitor may act to inhibit the cell cycle at any of the steps of the biological pathways shown in FIG. 16, as well as at other possible steps in other biological pathways. In addition, it should be understood that while a single cell cycle agent is often referred to, that this in fact should be understood to include two or more cell cycle agents, as more than one cell cycle agent may be utilized within the compositions, methods and/or devices described herein (e.g., two cell-cycle inhibitors may be selected that act on different steps shown in FIG. 16.

A wide variety of cell cycle inhibitory agents can be utilized, either with or without a carrier (e.g., a polymer or ointment or vector), in order to treat or prevent surgical adhesions. Representative examples of such agents include taxanes (e.g., paclitaxel (discussed in more detail below) and docetaxel) (Schiff et al., Nature 277:665-667,1979; Long and Fairchild, Cancer Research 54:4355-4361,1994; Ringel and Horwitz, J. Nat'l Cancer Inst. 83(4):288-291,1991; Pazdur et al., Cancer Treat Rev. 19(40):351-386,1993), Etanidazole, Nimorazole (B. A. Chabner and D. L. Longo. Cancer Chemotherapy and Biotherapy—Principles and Practice. Lippincott-Raven Publishers, New York, 1996, p.554), perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat Stud. Phys., Chem. Med. 40(2):153-61,1981), SR-2508 (Brown et al., Int. J. Radiat Oncol., Biol. Phys. 7(6):695-703, 1981), 2H-isoindolediones (J. A. Myers, 2H-lsoindolediones, their synthesis and use as radiosensitizers. U.S. Pat. No. 4,494,547, Jan. 22, 1985), chiral (((2-bromoethyl)-amino]methyl]-nitro-1H-imidazole-1-ethanol (V. G. Beylin, et al., Process for preparing chiral (((2-bromoethyl)-amino]methyl]-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and their use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec. 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al., Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and their use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, Jun. 1, 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil. Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12):3156-3174,1996), camptothecin (Ewend M. G. et al. Local delivery of chemotherapy and concurrent external beam radiotherapy prolongs survival in metastatic brain tumor models. Cancer Research 56(22):5217-5223, 1996) and paclitaxel (Tishler R. B. et al. Taxol: a novel radiation sensitizer. International Journal of Radiation Oncology and Biological Physics 22(3):613-617,1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA)₂Pt(DOLYM] and (DACH)Pt(DOLYM] cisplatin (Choi et al., Arch. Pharmacal Res. 22(2):151-156, 1999), Cis-(PtCl₂(4,7-H-5-methyl-7-oxo]1,2,4(triazolo[1,5-a]pyrimidine)₂] (Navarro et al., J. Med. Chem. 41(3):332-338, 1998), (Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356,1997), Pt(II) . . . Pt(II) (Pt₂(NHCHN(C(CH₂)(CH₃))]₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-(Pt(OAc)₂₁₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-(Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), C₁₋₉₇₃ cisplatin analogue (Yang et al., Int J. Oncol. 5(3):597-602, 1994), cis-diamminedichloroplatinum(II) and its analogues cis-1,1-cyclobutaned icarbosylato(2R)-2-methyl-1,4-butanediam-mineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1)31-5,1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-d ichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3,1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)CI) (Bellon & Lippard, Biophys. Chem. 35(2-3):179-88, 1990), 3H-cis-1,2-diaminocyclohexanedichloroplatinum(II) and 3H-cis-1,2-diaminocyclohexanemalonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45,1987), (NPr4)2((PtCL4).cis-(PtCl2—(NH2Me)2)) (Brammer et al., J. Chem. Soc., Chem. Commun. 6:443-5, 1987), aliphatic tricarboxylic acid platinum complexes (EPA 185225), cis-dichloro(amino acid)(tert-butylamine)platinum(II) complexes (Pasini & Bersanetti, Inorg. Chim. Acta 107(4):259-67, 1985); 4-hydroperoxycylcophosphamide (Ballard et al., Cancer Chemother. Pharmacol. 26(6):397-402, 1990), acyclouridine cyclophosphamide derivatives (Zakerinia et al., Helv. Chim. Acta 73(4):912-15, 1990), 1,3,2-dioxa- and -oxazaphosphorinane cyclophosphamide analogues (Yang et al., Tetrahedron 44(20):6305-14,1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1):31-7,1989), phenylketophosphamide cyclophosphamide analogues (Ludeman et al., J. Med. Chem. 29(5):716-27, 1986), ASTA Z-7557 cyclophosphamide analogues (Evans et al., Int. J. Cancer 34(6):883-90, 1984), 3-(1-oxy-2,2,6,6-tetramethyl-4-piperidinyl)cyclophosphamide (Tsui et al., J. Med. Chem. 25(9):1106-10, 1982), 2-oxobis(2-β-chloroethylamino)-4-,6-dimethyl-1,3,2-oxazaphosphorinane cyclophosphamide (Carpenter et al., Phosphorus Sulfur 12(3):287-93, 1982), 5-fluoro- and 5-chlorocyclophosphamide (Foster et al., J. Med. Chem. 24(12):1399-403, 1981), cis- and trans-4-phenylcyclophosphamide (Boyd et al., J. Med. Chem. 23(4):372-5, 1980), 5-bromocyclophosphamide, 3,5-dehydrocyclophosphamide (Ludeman et al., J. Med. Chem. 22(2):151-8, 1979), 4-ethoxycarbonyl cyclophosphamide analogues (Foster, J. Pharm. Sci. 67(5):709-10,1978), arylaminotetrahydro-2H-1,3,2-oxazaphosphorine 2-oxide cyclophosphamide analogues (Hamacher, Arch. Pharm. (Weinheim, Ger.) 310(5):J,428-34, 1977), NSC-26271 cyclophosphamide analogues (Montgomery & Struck, Cancer Treat. Rep. 60(4):J381-93, 1976), benzo annulated cyclophosphamide analogues (Ludeman & Zon, J. Med. Chem. 18(12):J1251-3, 1975), 6-trifluoromethylcyclophosphamide (Farmer & Cox, J. Med. Chem. 18(11):J1106-10, 1975), 4-methylcyclophosphamide and 6-methycyclophosphamide analogues (Cox et al., Biochem. Pharmacol. 24(5):J599-606, 1975); FCE 23762 doxorubicin derivative (Quaglia et al., J. Liq. Chromatogr. 17(18):3911-3923, 1994), annamycin (Zou et al., J. Pharm. Sci. 82(11):1151-1154,1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116,1998), 2-pyrrolinodoxorubicin (Nagy et al., Proc. Nat'l. Acad. Sci. U.S.A. 95(4):1794-1799, 1998), disaccharide doxorubicin analogues (Arcamone et al., J. Nat'l Cancer Inst. 89(16):1217-1223,1997), 4-demethoxy-7-O-(2,6-dideoxy-4-O-(2,3,6-trideoxy-3-amino-α-L-lyxo-hexopyranosyl)-α-L-lyxo-hexopyranosyl]adriamicinone doxorubicin disaccharide analog (Monteagudo et al., Carbohydr. Res. 300(1):11-16, 1997), 2-pyrrolinodoxorubicin (Nagy et al., Proc. Nat'l Acad. Sci. U.S. A. 94(2):652-656, 1997), morpholinyl doxorubicin analogues (Duran et al., Cancer Chemother. Pharmacol. 38(3):210-216, 1996), enaminomalonyl-α-alanine doxorubicin derivatives (Seitz et al., Tetrahedron Lett. 36(9): 1413-16,1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9,1991), 4-demethoxy-3′-N-trifluoroacetyidoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7,1988), 4′-deoxydoxorubicin (Schoelzel et al., Leuk. Res. 10(12):1455-9,1986), 4-demethyoxy-4′-o-methyldoxorubicin (Giuliani et al., Proc. Int. Congr. Chemother. 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52,1978), SM 5887 (Pharma Japan 1468:20,1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43(6):1337-44, 1992), azo and azoxy misonidazole derivatives (Gattavecchia & Tonelli, Int. J. Radiat Biol. Relat. Stud. Phys., Chem. Med. 45(5):469-77, 1984); RB90740 (Wardman et al., Br. J. Cancer, 74 Suppl. (27):S70-S74,1996); 6-bromo and 6-chloro-2,3-dihydro-1,4-benzothiazines nitrosourea derivatives (Rai et al., Heterocycl. Commun. 2(6):587-592,1996), diamino acid nitrosourea derivatives (Dulude et al., Bioorg. Med. Chem. Lett. 4(22):2697-700, 1994; Dulude et al., Bioorg. Med. Chem. 3(2):151-60,1995), amino acid nitrosourea derivatives (Zheleva et al., Pharmazie 50(1):25-6, 1995), 3′,4′-didemethoxy-3′,4′-dioxo-4-deoxypodophyllotoxin nitrosourea derivatives (Miyahara et al., Heterocycles 39(1):361-9,1994), ACNU (Matsunaga et al., Immunopharmacology 23(3):199-204,1992), tertiary phosphine oxide nitrosourea derivatives (Guguva et al., Pharmazie 46(8):603,1991), sulfamerizine and sulfamethizole nitrosourea derivatives (Chiang et al., Zhonghua Yaozue Zazhi 43(5):401-6,1991), thymidine nitrosourea analogues (Zhang et al., Cancer Commun. 3(4):119-26, 1991), 1,3-bis(2-chloroethyl)-1-nitrosourea (August et al., Cancer Res. 51(6):1586-90,1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9):1311-16,1989), pyrimidine (II) nitrosourea derivatives (Wei et al., Chung-hua Yao Hsueh Tsa Chih 41(1):19-26, 1989), CGP 6809 (Schieweck et al., Cancer Chemother. Pharmacol. 23(6):341-7, 1989), B-3839 (Prajda et al., In Vivo 2(2):151-4, 1988), 5-halogenocytosine nitrosourea derivatives (Chiang & Tseng, T'ai-wan Yao Hsueh Tsa Chih 38(1):37-43, 1986),1-(2-chloroethyl)-3-isobutyl-3-(β-maltosyl)-1-nitrosourea (Fujimoto & Ogawa, J. Pharmacobio-Dyn. 10(7):341-5, 1987), sulfur-containing nitrosoureas (Tang et al., Yaoxue Xuebao 21(7):502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33(11):969-77,1985), CNCC, RFCNU and chlorozotocin (Mena et al., Chemotherapy (Basel) 32(2):131-7,1986), CNUA (Edanami et al., Chemotherapy (Tokyo) 33(5):455-61,1985), 1-(2-chloroethyl)-3-isobutyl-3-(β-maltosyl)-1-nitrosourea (Fujimoto & Ogawa, Jpn. J. Cancer Res. (Gann) 76(7):651-6, 1985), choline-like nitrosoalkylureas (Belyaev et al., Izv. Akad. NAUK SSSR, Ser. Khim. 3:553-7,1985), sucrose nitrosourea derivatives (JP 84219300), sulfa drug nitrosourea analogues (Chiang et al., Proc. Nat'l Sci. Counc., Repub. China, Part A 8(1):18-22, 1984), DONU (Asanuma et al., J. Jpn. Soc. Cancer Ther. 17(8):2035-43,1982), N,N′-bis (N-(2-chloroethyl)-N-nitrosocarbamoyl)cystamine (CNCC) (Blazsek et al., Toxicol. Appl. Pharmacol. 74(2):250-7, 1984), dimethylnitrosourea (Krutova et al., Izv. Akad. NAUK SSSR, Ser. Biol. 3:439-45, 1984), GANU (Sava & Giraldi, Cancer Chemother. Pharmacol. 10(3):167-9, 1983), CCNU (Capelli et al., Med., Biol., Environ. 11(1): 111-16,1983), 5-aminomethyl-2′-deoxyuridine nitrosourea analogues (Shiau, Shih Ta Hsueh Pao (Taipei) 27:681-9,1982), TA-077 (Fujimoto & Ogawa, Cancer Chemother. Pharmacol. 9(3):134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.):165-74, 1981), thiocolchicine nitrosourea analogues (George, Shih Ta Hsueh Pao (Taipei) 25:355-62,1980), 2-chloroethyl-nitrosourea (Zeller & Eisenbrand, Oncology 38(1):39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7(8):1393-401,1980), N-deacetylmethyl thiocolchicine nitrosourea analogues (Lin et al., J. Med. Chem. 23(12):1440-2, 1980), pyridine and piperidine nitrosourea derivatives (Crider et al., J. Med. Chem. 23(8):848-51,1980), methyl-CCNU (Zimber& Perk, Refu. Vet 35(1):28, 1978), phensuzimide nitrosourea derivatives (Crider et al., J. Med. Chem. 23(3):324-6, 1980), ergoline nitrosourea derivatives (Crider et al., J. Med. Chem. 22(1):32-5, 1979), glucopyranose nitrosourea derivatives (JP 78 95917), 1-(2-chloroethyl)-3-cyclohexyl-1-nitrosourea (Farmer et al., J. Med. Chem. 21(6):514-20, 1978), 4-(3-(2-chloroethyl)-3-nitrosoureid-o)-cis-cyclohexanecarboxylic acid (Drewinko et al., Cancer Treat. Rep. 61(8):J1513-18,1977), RPCNU (ICIG 1163) (Larnicol et al., Biomedicine 26(3):J176-81, 1977), IOB-252 (Sorodoc et al., Rev. Roum. Med. Virol. 28(1):J55-61, 1977), 1,3-bis(2-chloroethyl)-1-nitrosourea (BCNU) (Siebert & Eisenbrand, Mutat. Res. 42(1):J45-50, 1977), 1-tetrahydroxycyclopentyl-3-nitroso-3-(2-chloroethyl)-urea (U.S. Pat. No. 4,039,578), d-1-1-(D-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7,1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7,1981); indoline ring and a modified ornithine or glutamic acid-bearing methotrexate derivatives (Matsuoka et al., Chem. Pharm. Bull. 45(7):1146-1150,1997), alkyl-substituted benzene ring C bearing methotrexate derivatives (Matsuoka et al., Chem. Pharm. Bull. 44(12):2287-2293,1996), benzoxazine or benzothiazine moiety-bearing methotrexate derivatives (Matsuoka et al., J. Med. Chem. 40(1):105-111,1997), 10-deazaminopterin analogues (DeGraw et al., J. Med. Chem. 40(3):370-376, 1997), 5-deazaminopterin and 5,10-dideazaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm., Biopharm. Pharm. Technol., 563-4,1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2):101-2,1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9,1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7,1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582,1989), hydroxymethylmethotrexate (DE 267495), γ-fluoromethotrexate (McGuire et al., Cancer Res. 49(16):4517-25,1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87,1988), 5-methyl-5-deaza methotrexate analogues (U.S. Pat. No. 4,725,687), Nδ-acyl-Na-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24,1987), methotrexate-γ-dimyristoylphophatidylethanolamine (Kinsky et al., Biochim. Biophys. Acta 917(2):211-18,1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8,1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92,1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9,1986), ω-diaminoalkanoid acid-containing methotrexate analogues (McGuire et al., Biochem. Pharmacol. 35(15):2607-13,1986), polyglutamate methotrexate derivatives (Kamen & Winick, Methods Enzymol. 122(Vitam. Coenzymes, Pt. G):339-46, 1986), 5-methyl-5-deaza analogues (piper et al., J. Med. Chem. 29(6):1080-7, 1986), quinazoline methotrexate analogue (Mastropaolo et al., J. Med. Chem. 29(1):155-8,1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.-Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9,1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10,1980), halogentated methotrexate derivatives (Fox, JNCI 58(4):J955-8, 1977), 8-alkyl-7,8-dihydro analogues (Chaykovsky et al., J. Med. Chem. 20(10):J11323-7,1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19):3145-3146,1998), 5-fluorouracil derivatives with 1,4-oxaheteroepane moieties (Gomez et al., Tetrahedron 54(43):13295-13312,1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9,1992), A-OT-fluorouracil (Zhang et al., Zongguo Yiyao Gongye Zazhi 20(11):513-15,1989), N4-trimethoxybenzoyl-5′-deoxy-5-fluorocytidine and 5′-deoxy-5-fluorouridine (Miwa et al., Chem. Pharm. Bull. 38(4):998-1003, 1990),1-hexylcarbamoyl-5-fluorouracil (Hoshi et al., J. Pharmacobio-Dun. 3(9):478-81,1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31,1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67,1984); N-substituted deacetylvinblastine amide (vindesine) sulfates (Conrad et al., J. Med. Chem. 22(4):391-400,1979); and Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Leff. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

Within one preferred embodiment of the invention, the cell cycle inhibitor is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93:2325,1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL (Bristol-Myers Squibb Company, New York, N.Y.), TAXOTERE (Aventis Pharmaceuticals, France), docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361,1994; Ringel and Horwitz, J. Nat'l Cancer Inst. 83(4):288-291, 1991; Pazdur et al., Cancer Treat. Rev. 19(4):351-386,1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998,1991; J. Natural Prod. 57(10):1404-1410, 1994; J. Natural Prod. 57(11):1580-1583,1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogues with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the Cell Cycle Inhibitor is a taxane having the formula (C1):

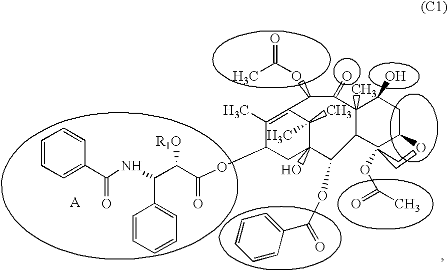

where the gray-highlighted portions may be substituted and the non-highlighted portion is the taxane core. A side-chain (labeled “A” in the diagram) is desirably present in order for the compound to have good activity as a Cell Cycle Inhibitor. Examples of compounds having this structure include paclitaxel (Merck Index entry 7117), docetaxol (Taxotere, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2):

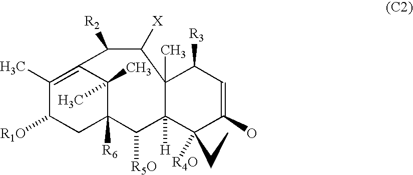

wherein X may be oxygen (paclitaxel), hydrogen (9-deoxy derivatives), thioacyl, or dihydroxylprecursors; R₁ is selected from paclitaxel or taxotere side chains or alkanoyl of the formula (C3)

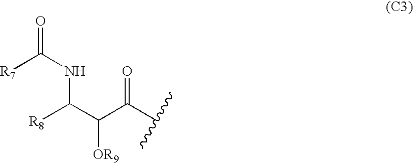

wherein R₇ is selected from hydrogen, alkyl, phenyl, alkoxy, amino, phenoxy (substituted or unsubstituted); R₈ is selected from hydorgen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydroxyl, alkoyl, alkanoyloxy, aminoalkanoyloxy, and peptidyalkanoyloxy; R₃ is selected from hydrogen or oxygen-containing groups, such as hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as Cell Cycle Inhibitors in the present invention are disclosed in PCT International Patent Application No. WO 93/10076. As disclosed in this publication, the analog or derivative should have a side chain attached to the taxane nucleus at C₁₃, as shown in the structure below (formula C4), in order to confer antitumor activity to the taxane.

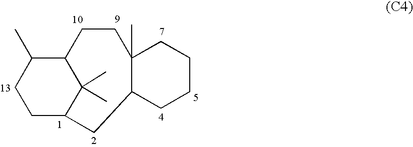

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based Cell Cycle Inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a Cell Cycle Inhibitor by acting as a anti-microtuble agent, and more specifically as a stabilizer. These compounds have been shown useful in the treatment of proliferative disorders, including: non-small cell (NSC) lung; small cell lung; breast; prostate; cervical; endometrial; head and neck cancers.

In another aspect, the Cell Cycle Inhibitor is a Vinca Alkaloid. Vinca alkaloids have the following general structure. They are indole-dihydroindole dimers.

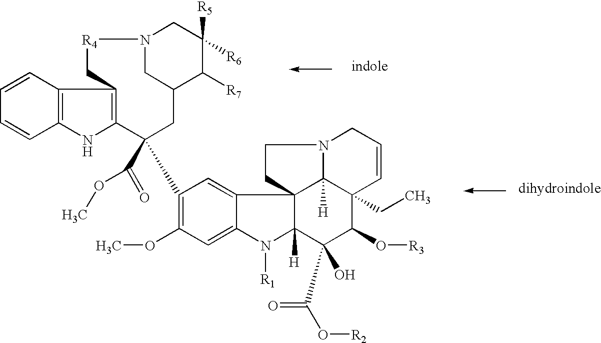

As disclosed in U.S. Pat. Nos. 4,841,045 and 5,030,620, R₁ can be a formyl or methyl group or alternately H. R₁ could also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group such as maleoyl amino acid. R₃ could also be substituted to form an alkyl ester which may be further substituted. R₄ may be —CH₂— or a single bond. R₅ and R₆ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary Vinca Alkaloids are vinblastine, vincristine, vincristine sulfate, vindesine, and vinorelbine, having the structures:

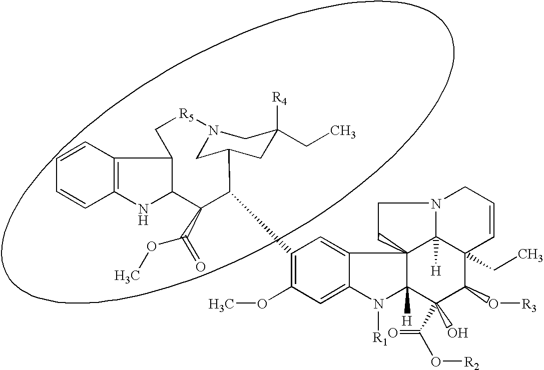

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogues typically require the side group (shaded area) in order to have activity. These compounds are thought to act as Cell Cycle Inhibitors by functioning as anti-microtubole agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the Cell Cycle Inhibitor is Camptothecin, or an anolog or derivative thereof. Camptothecins have the following general structure.

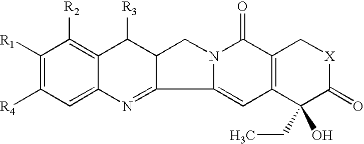

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

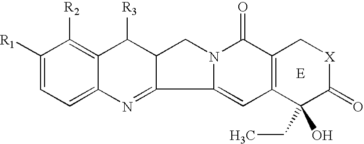

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as Cell Cycle Inhibitors, where they function as Topoisomerase I Inhibitors and/or DNA cleavage agents. They have been shown useful in the treatment of proliferative disorders, including, for example, NSC lung; small cell lung; and cervical cancers.

In another aspect, the Cell Cycle Inhibitor is a Podophyllotoxin, or a derivative or an analog thereof. Exemplary compounds of this type are Etoposide or Teniposide, which have the following structures:

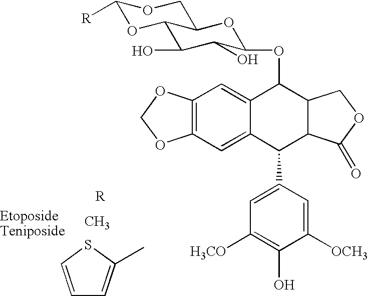

These compounds are thought to function as Cell Cycle Inhibitors by being Topoisomerase II Inhibitors and/or by DNA cleaving agents. They have been shown useful as antiproliferative agents in, e.g., small cell lung, prostate, and brain cancers, and in retinoblastoma.

In another aspect, the Cell Cycle Inhibitor is an Anthracycline. Anthracyclines have the following general structure, where the R groups may be a variety of organic groups:

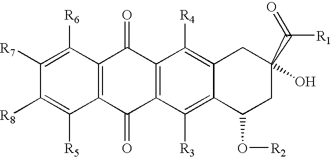

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

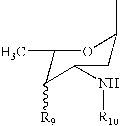

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary Anthracycline are Doxorubicin, Daunorubicin, Idarubicin, Epirubicin, Pirarubicin, Zorubicin, and Carubicin. Suitable compounds have the structures:

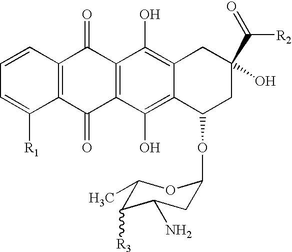

| | R₁ | R₂ | R₃ |
|---|---|---|---|
| Doxorubicin: | OCH₃ | CH₂OH | OH out of ring plane |
| Epirubucin: | OCH₃ | CH₂OH | OH in ring plane |
| (4″ epimer of | | | |
| doxorubucin) | | | |
| Deunorubicin: | OCH₃ | CH₃ | OH out of ring plane |
| Idarubicin: | H | CH₃ | OH out of ring plane |
| Pirarubicin | OCH₃ | OH | A |
| Zorubicin | OCH₃ | ═N—NHC(O)C₆H₅ | B |
| Cerubicin | OH | CH₃ | B |

| | |
|---|---|
| 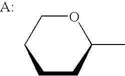 | 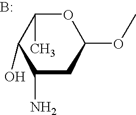 |

Other suitable Anthracyclines are Anthramycin, Mitoxantrone, Menogaril, Nogalamycin, Aclacinomycin A, Olivomycin A, Chromomycin A₃, and Plicamycin having the structures:

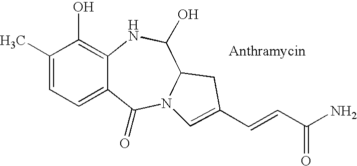

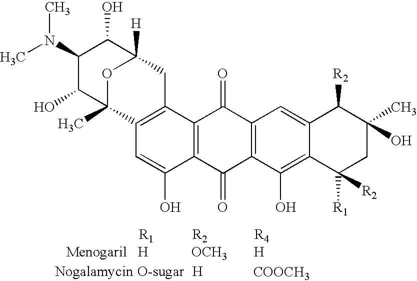

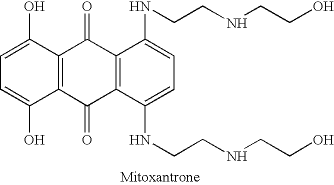

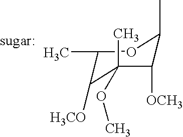

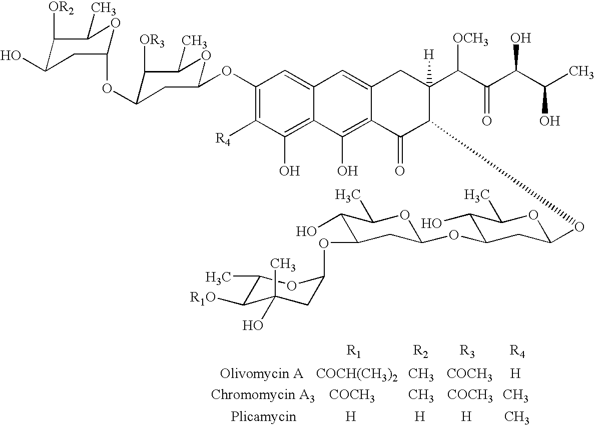

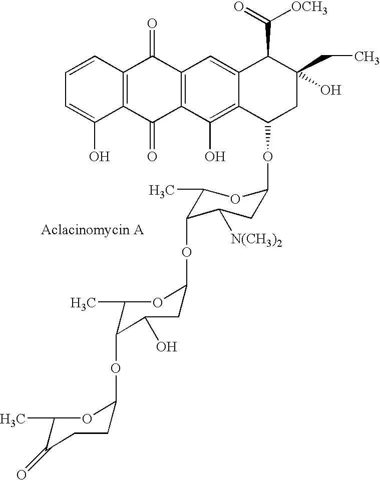

These compounds are thought to function as Cell Cycle Inhibitors by being Topoisomerase Inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the Cell Cycle Inhibitor is a Platinum compound. In general, suitable platinum complexes may be of Pt(II) or Pt(IV) and have this basic structure:

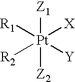

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

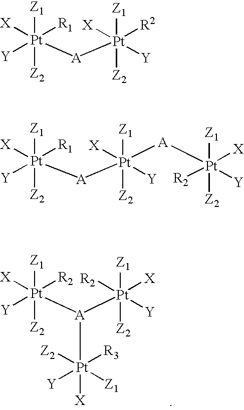

Exemplary Platinum compound are Cisplatin, Carboplatin, Oxaliplatin, and Miboplatin having the structures:

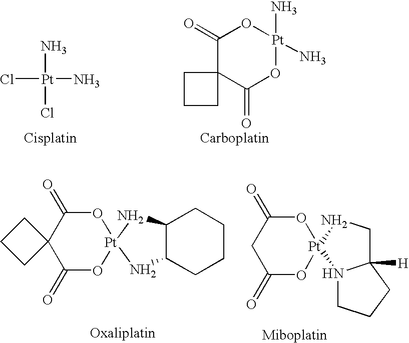

These compounds are thought to function as Cell Cycle Inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the Cell Cycle Inhibitor is a Nitrosourea. Nitrosourease have the following general structure (C5), where typical R groups are shown below.

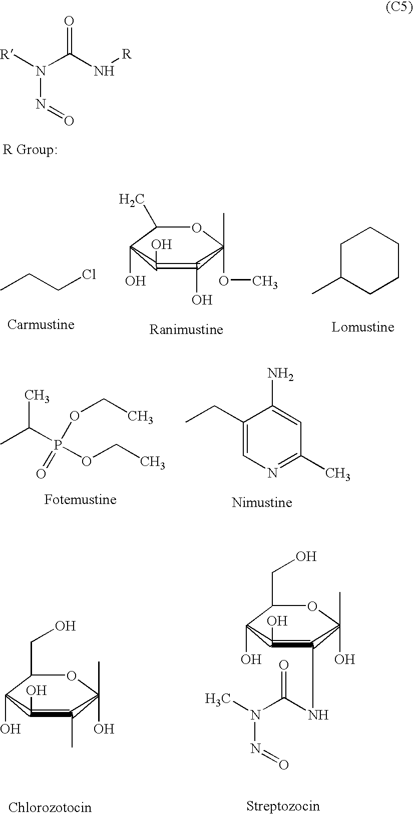

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₄). Z has the following structure: -alkylene-N—R₁R₂, where R₁ and R₂ may be the same or different members of the following group: lower alkyl (C₁₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., Methyl 2′-(N-(N-(2-chloroethyl)-N-nitroso-carbamoyl]-glycyl]amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylica acid or CONH₂ group.

Exemplary Nitrosourea are BCNU (Carmustine), Methyl-CCNU (Semustine), CCNU (Lomustine), Ranimustine, Nimustine, Chlorozotocin, Fotemustine, Streptozocin, and Streptozocin, having the structures:

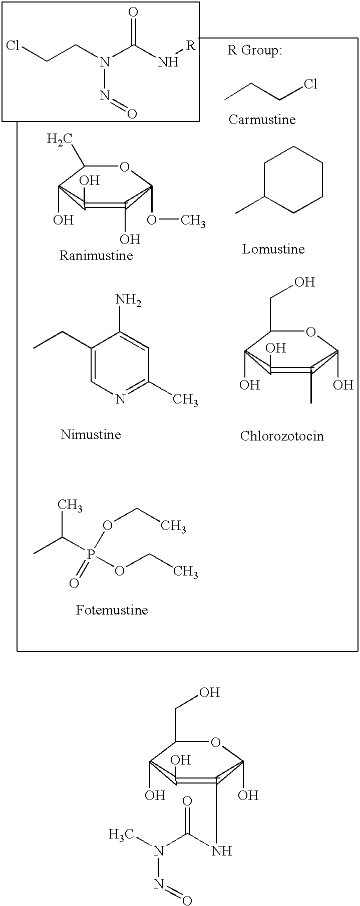

These nitrosourea compounds are thought to function as Cell Cycle Inhibitor by binding to DNA, that is, by functioning as DNA alkylating agents. These Cell Cycle Inhibitors have been shown useful in treating cell proliferative disorders such as, for example, islet cell; small cell lung; melanoma; and brain cancers.

In another aspect, the Cell Cycle Inhibitor is a Nitroimidazole, where exemplary Nitroimidazoles are Metronidazole, Benznidazole, Etanidazole, and Misonidazole, having the structures:

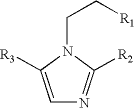

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂OH | NO₂ | H |

Suitable nitroimidazole compounds are disclosed in, e.g., U.S. Pat. Nos. 4,371,540 and 4,462,992.

In another aspect, the Cell Cycle Inhibitor is a Folic acid antagonist, such as Methotrexate or derivatives or analogues thereof, including Edatrexate, Trimetrexate, Raltitrexed, Piritrexim, Denopterin, Tomudex, and Pteropterin. Methotrexate analogues have the following general structure:

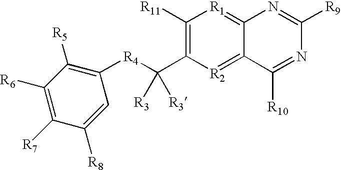

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure:

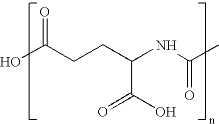

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

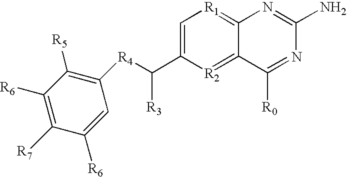

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | N(CH₂CH₃) | H | H | A (n = 1) | H |
| Trimetrexate | NH₂ | N | C(CH₃) | H | NH | H | OCH₃ | OCH₃ | OCH₃ |
| Pteropterin | NH₂ | N | N | H | N(CH₃) | H | H | A (n = 3) | H |
| Denopterin | OH | N | N | CH₃ | N(CH₃) | H | N | A (n = 1) | H |
| Piritrexim | NH₂ | N | C(CH₃)H | single bond | OCH₃ | H | H | OCH₃ | H |

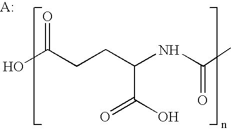

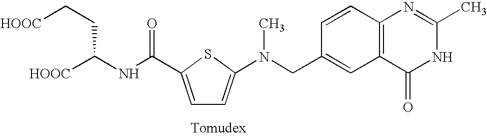

These compounds are thought to function as Cell Cycle Inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the Cell Cycle Inhibitor is a Cytidine Analog, such as Cytarabine or derivatives or analogues thereof, including Enocitabine, FMdC ((E(−2′-deoxy-2′-(fluoromethylene)cytidine), Gemcitabine, 5-Azacitidine, Ancitabine, and 6-Azauridine. Exemplary compounds have the structures:

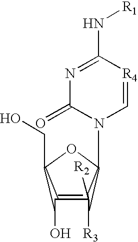

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

| | |
|---|---|
| 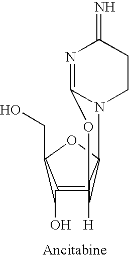 | 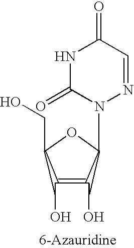 |

These compounds are thought to function as Cell Cycle Inhibitors as acting as antimetabolites of pyrimidine. These compounds have been shown useful in the treatment of cell proliferative disorders including, for example, pancreatic, breast, cervical, NSC lung, and bile duct cancers.

In another aspect, the Cell Cycle Inhibitor is a Pyrimidine analog. In one aspect, the Pyrimidine analogues have the general structure:

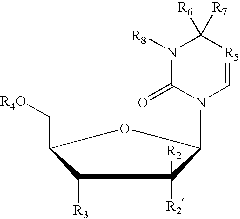

wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆=—NH—R₁ and R₇═H. R₈ is H or R₇ and R₈ together can form a double bond or R₈ can be X, where X is:

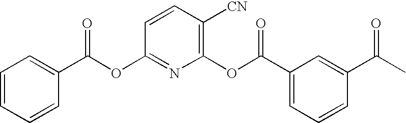

Specific pyrimidine analogues are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the Cell Cycle Inhibitor is a Fluoro-pyrimidine Analog, such as 5-Fluorouracil, or an analog or derivative thereof, including Carmofur, Doxifluridine, Emitefur, Tegafur, and Floxuridine. Exemplary compounds have the structures:

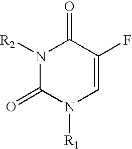

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | | H |

| | |
|---|---|
| 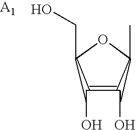 | 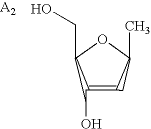 |

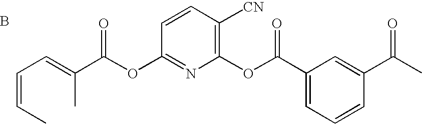

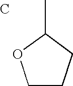

Other suitable Fluoropyrimidine Analogues include 5-FudR (5-fluoro-deoxyuridine), or an analog or derivative thereof, including 5-iododeoxyuridine (5-IudR), 5-bromodeoxyuridine (5-BudR), Fluorouridine triphosphate (5-FUTP), and Fluorodeoxyuridine monophosphate (5-dFUMP). Exemplary compounds have the structures:

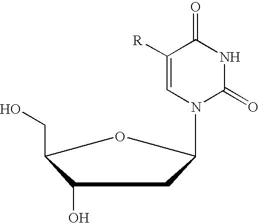

5-Fluoro-2′-deoxyuridine: R═F

5-Bromo-2′-deoxyuridine: R═Br

5-Iodoo-2′-deoxyuridine: R═I

These compounds are thought to function as Cell Cycle Inhibitors by serving as antimetabolites of pyrimidine.

In another aspect, the Cell Cycle Inhibitor is a Purine Analog. Purine analogues have the following general structure:

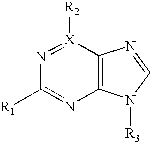

wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R2 is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogues of the type shown in the following formula:

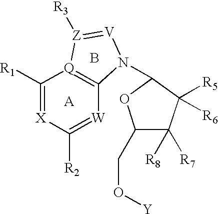

wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogues include 6-Mercaptopurine, Thiguanosine, Thiamiprine, Cladribine, Fludaribine, Tubercidin, Puromycin, Pentoxyfilline; where these compounds may optionally be phosphorylated. Exemplary compounds have the structures:

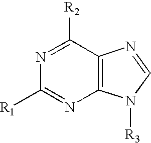

| | R₁ | R₂ | R₃ | | |
|---|---|---|---|---|---|
| 6-Mercaptopurine Thioguanosine Thiamiprine | H NH₂NH₂ | SH SH A | H B₁H | 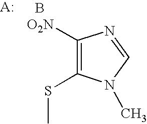 | 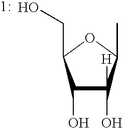 |
| Cladribine Fludarabine Puromycin | Cl F H | NH₂NH₂N(CH₃)₂ | B₂B₃B₄ | 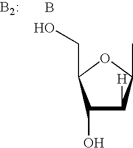 | 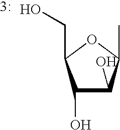 |

| | | | | |
|---|---|---|---|---|
| Tubercidine | H | NH₂ | B₁ | 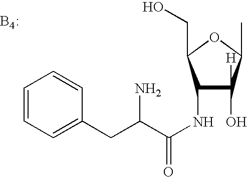 |

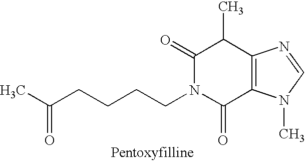

These compounds are thought to function as Cell Cycle Inhibitors by serving as antimetabolites of purine.

In another aspect, the Cell Cycle Inhibitor is a Nitrogen Mustard. Many suitable Nitrogen Mustards are known and are suitably used as a Cell Cycle Inhibitor in the present invention. Suitable nitrogen mustards are also known as cyclophosphamides.

A preferred nitrogen mustard has the general structure:

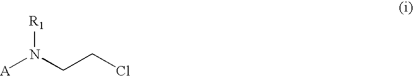

Where A is:

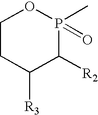

or —CH₃ or other alkane, or chloronated alkane, typically CH₂CH(CH₃)Cl, or a polycyclic group such as B, or a substituted phenyl such as C or a heterocyclic group such as D.

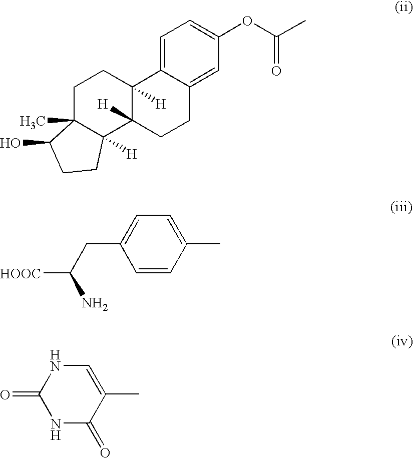

Suitable nitrogen mustards are disclosed in U.S. Pat. No. 3,808,297, wherein A is:

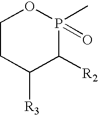

R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure:

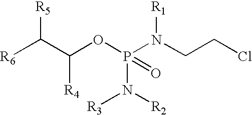

wherein R₁ is H or CH₂CH₂Cl, and R₂₋₆ are various substituent groups.

Exemplary nitrogen mustards include methylchloroethamine, and analogues or derivatives thereof, including methylchloroethamine oxide hydrohchloride, Novembichin, and Mannomustine (a halogenated sugar). Exemplary compounds have the structures:

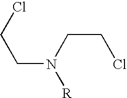

| | R | |
|---|---|---|
| Mechlorethanime | CH₃ | 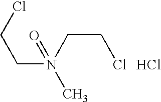 |
| Novembichin | CH₂CH(CH₃)Cl | Mechlorethanime Oxide HCl |

The Nitrogen Mustard may be Cyclophosphamide, Ifosfamide, Perfosfamide, or Torofosfamide, where these compounds have the structures:

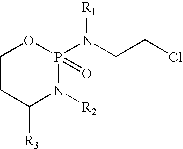

| | R₁ | R₂ | R₃ |
|---|---|---|---|
| Cyclophosphamide | H | CH₂CH₂Cl | H |
| fosfamide | CH₂CH₂Cl | H | H |
| Perfosfamide | CH₂CH₂Cl | H | OOH |
| Torofosfamide | CH₂CH₂Cl | CH₂CH₂Cl | H |

The Nitrogen Mustard may be Estramustine, or an analog or derivative thereof, including Phenesterine, Prednimustine, and Estramustine PO₄. Thus, suitable nitrogen mustard type Cell Cycle Inhibitors of the present invention have the structures:

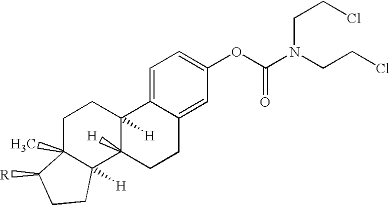

| | R |
|---|---|
| Estramustine | OH |
| Phenesterine | C(CH₃)(CH₂)₃CH(CH₃)₂ |

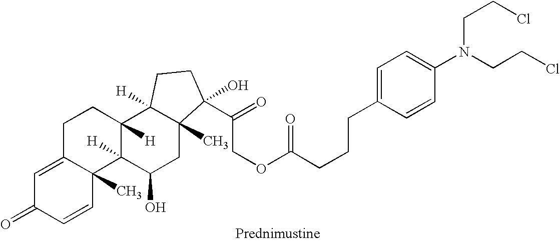

The Nitrogen Mustard may be Chlorambucil, or an analog or derivative thereof, including Melphalan and Chlormaphazine. Thus, suitable nitrogen mustard type Cell Cycle Inhibitors of the present invention have the structures:

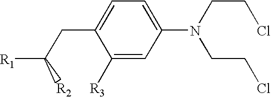

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together |
| | | forms a |
| | | benzene ring |

The Nitrogen Mustard may be Uracil Mustard, which has the structure:

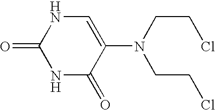

The Nitrogen Mustards are thought to function as Cell Cycle Inhibitors by serving as alkylating agents for DNA.

The Cell Cycle Inhibitor of the present invention may be a Hydroxyurea. Hydroxyureas have the following general structure:

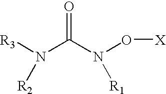

Suitable Hydroxyureas are disclosed in, for example, U.S. Pat. No. 6,080,874, wherein R₁ is:

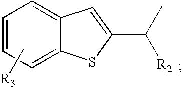

and R₂ is an alkyl group having 1-4 carbons and R₃ is one of H, acyl, methyl, ethyl, and mixtures thereof, such as a methylether.

Other suitable Hydroxyureas are disclosed in, e.g., U.S. Pat. No. 5,665,768, wherein R₁ is a cycloalkenyl group, for example N-(3-(5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable Hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable Hydroxyureas are disclosed in, e.g., U.S. Pat. No. 5,066,658, wherein R₂ and R₃ together with the adjacent nitrogen form:

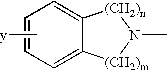

wherein m is 1 or 2, n is 0-2 and Y is an alkyl group.

In one aspect, the hydroxy urea has the structure:

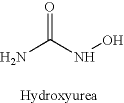

Hydroxyureas are thought to function as Cell Cycle Inhibitors by serving to inhibit DNA synthesis.

In another aspect, the Cell Cycle Inhibitor is a Belomycin, such as Bleomycin A₂, which have the structures:

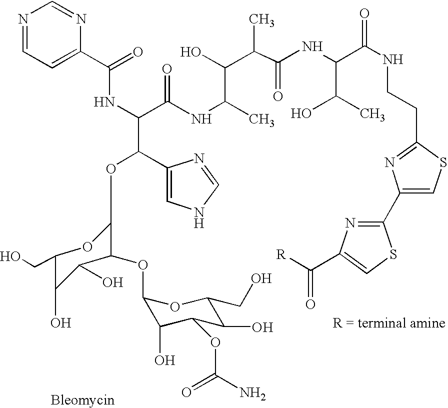

Bleomycin A₂: R═(CH₃)₂S⁺(CH₂)₃NH—

Belomycins are thought to function as Cell Cycle Inhibitors by cleaving DNA. They have been shown useful in the treatment of cell proliferative disorder such as, e.g., penile cancer.

In another aspect, the Cell Cycle Inhibitor is a Mytomicin, such as Mitomycin C, or an analog or derivative thereof, such as Porphyromycin. Suitable compounds have the structures:

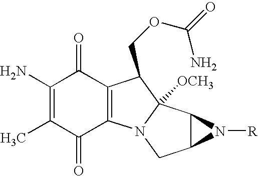

| | R |
|---|---|
| Mitomycin C | H |
| Porphyromycin | CH₃ |
| (N-methyl Mitomycin C) | |

These compounds are thought to function as Cell Cycle Inhibitors by serving as DNA alkylating agents.

In another aspect, the Cell Cycle Inhibitor is an Alkyl sulfonate, such as Busulfan, or an analog or derivative thereof, such as Treosulfan, Improsulfan, Piposulfan, and Pipobroman. Exemplary compounds have the structures:

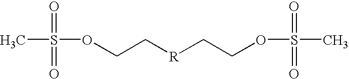

| | R |
|---|---|
| Busulfan | single bond |
| Improsulfan | —CH₂—NH—CH₂— |
| Piposulfan | 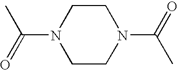 |

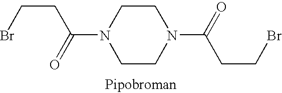

These compounds are thought to function as Cell Cycle Inhibitors by serving as DNA alkylating agents.

In another aspect, the Cell Cycle Inhibitor is a Benzamide. In yet another aspect, the Cell Cycle Inhibitor is a Nicotinamide. These compounds have the basic structure:

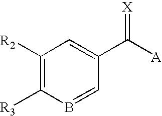

wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-Ethyl (see, e.g., U.S. Pat. No. 5,215,738).

Suitable Benzamide compounds have the structures:

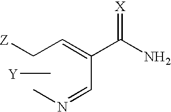

Benzamides

X=O or S

Y=H, OR, CH₃, acetoxy

Z=H, OR, SR, NHR

R=alkyl group

where additional compounds are disclosed in U.S. Pat. No. 5,215,738, (listing some 32 compounds).

Suitable Nicotinamide compounds have the structures:

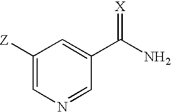

Nicotinamides

X=O or S

Z=H, OR, SR, NHR

R=alkyl group

where additional compounds are disclosed in U.S. Pat. No. 5,215,738 (listing some 58 compounds, e.g., 5-OH nicotinamide, 5-aminonicotinamide, 5-(2,3-dihydroxypropoxy) nicotinamide), and compounds having the structures:

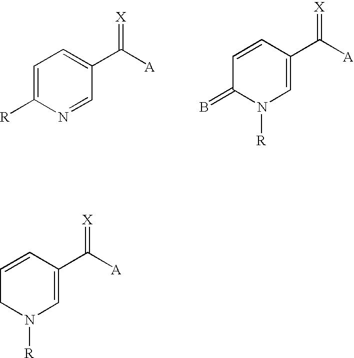

Nicotinamides

X=O or S (only O is described)

A=OH, NH₂, alkoxy

B=O

R=alkyl or aryl group

and U.S. Pat. No. 4,258,052 (listing some 46 compounds, e.g., 1-methyl-6-keto-1,6-dihydronicotinic acid).

In one aspect, the Cell Cycle Inhibitor is a Tetrazine Compound, such as Temozolomide, or an analog or derivative thereof, including Dacarbazine. Suitable compounds have the structures:

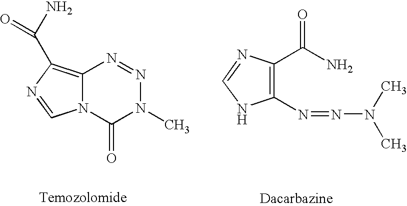

Another suitable Tetrazine Compound is Procarbazine, including HCl and HBr salts, having the structure:

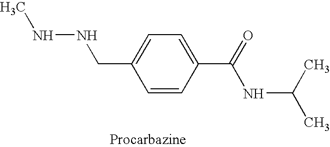

In another aspect, the Cell Cycle Inhibitor is Actinomycin D, or other members of this family, including Dactinomycin, Actinomycin C₁, Actinomycin C₂, Actinomycin C₃, and Actinomycin F₁. Suitable compounds have the structures:

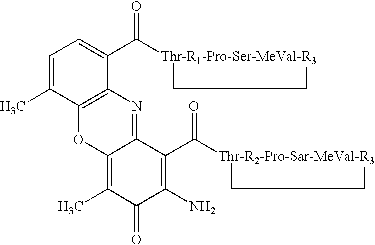

| | R₁ | R₂ | R₃ |
|---|---|---|---|
| Actinomycin D (C₁) | D-Val | D-Val | single bond |
| Actinomycin C₂ | D-Val | D-Alloisoleucine | O |
| Actinomycin C₃ | D-Alloisoleucine | D-Alloisoleucine | O |

In another aspect, the Cell Cycle Inhibitor is an Aziridine compound, such as Benzodepa, or an analog or derivative thereof, including Meturedepa, Uredepa, and Carboquone. Suitable compounds have the structures:

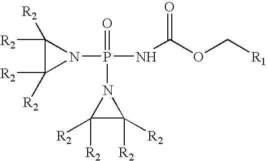

| | R₁ | R₂ | |
|---|---|---|---|
| Benzodepa Meturedepa Uredepa | phenyl CH₃CH₃ | H CH₃H | 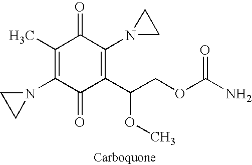 |

In another aspect, the Cell Cycle Inhibitor is Halogenated Sugar, such as Mitolactol, or an analog or derivative thereof, including Mitobronitol and Mannomustine. Suitable compounds have the structures:

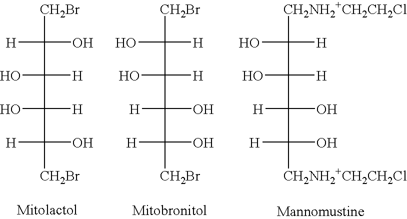

In another aspect, the Cell Cycle Inhibitor is a Diazo compound, such as Azaserine, or an analog or derivative thereof, including 6-diazo-5-oxo-L-norleucine and 5-diazouracil (also a pyrimidine analog). Suitable compounds have the structures:

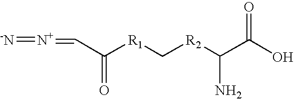

| | R₁ | R₂ |
|---|---|---|
| Azaserine | O | single bond |
| 6-diazo-5-oxo-L-norleucine | single bond | CH₂ |

Other compounds that may serve as Cell Cycle Inhibitors according to the present invention are Pazelliptine; Wortmannin; Metoclopramide; RSU; Buthionine sulfoxime; Tumeric; Curcumin; AG337, a thymidylate synthase inhibitor; Levamisole; Lentinan, a polysaccharide; Razoxane, an EDTA analog; Indomethacin; Chlorpromazine; α and β interferon; MnBOPP; Gadolinium texaphyrin; 4-amino-1,8-naphthalimide; Staurosporine derivative of CGP; and SR-2508.

Thus, in one aspect, the Cell Cycle Inhibitor is a DNA alkylating agent. In another aspect, the Cell Cycle Inhibitor is an anti-microtubule agent. In another aspect, the Cell Cycle Inhibitor is a Topoisomerase inhibitor. In another aspect, the Cell Cycle Inhibitor is a DNA cleaving agent. In another aspect, the Cell Cycle Inhibitor is an antimetabolite. In another aspect, the Cell Cycle Inhibitor functions by inhibiting adenosine deaminase (e.g., as a purine analog). In another aspect, the Cell Cycle Inhibitor functions by inhibiting purine ring synthesis and/or as a nucleotide interconversion inhibitor (e.g., as a purine analog such as mercaptopurine). In another aspect, the Cell Cycle Inhibitor functions by inhibiting dihydrofolate reduction and/or as a thymidine monophosphate block (e.g., methotrexate). In another aspect, the Cell Cycle Inhibitor functions by causing DNA damage (e.g., Bleomycin). In another aspect, the Cell Cycle Inhibitor functions as a DNA intercalation agent and/or RNA synthesis inhibition (e.g., Doxorubicin). In another aspect, the Cell Cycle Inhibitor functions by inhibiting pyrimidine synthesis (e.g., N-phosphonoacetyl-L-Aspartate). In another aspect, the Cell Cycle Inhibitor functions by inhibiting ribonucleotides (e.g., hydroxyurea). In another aspect, the Cell Cycle Inhibitor functions by inhibiting thymidine monophosphate (e.g., 5-fluorouracil). In another aspect, the Cell Cycle Inhibitor functions by inhibiting DNA synthesis (e.g., Cytarabine). In another aspect, the Cell Cycle Inhibitor functions by causing DNA adduct formation (e.g., platinum compounds). In another aspect, the Cell Cycle Inhibitor functions by inhibiting protein synthesis (e.g., L-Asparginase). In another aspect, the Cell Cycle Inhibitor functions by inhibiting microtubule function (e.g., taxanes). In another aspect, the Cell Cycle Inhibitors acts at one or more of the steps in the biological pathway shown in FIG. 16.

Additional Cell Cycle Inhibitors useful in the present invention, as well as a discussion of their mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561 (all of which, as noted above, are incorporated by reference in their entirety) Numerous polypeptides, proteins and peptides, as well as nucleic acids that encode such proteins, can also be used therapeutically as cell cycle inhibitors. This is accomplished by delivery by a suitable vector or gene delivery vehicle which encodes a cell cycle inhibitor (Walther & Stein, Drugs 60(2):249-71, August 2000; Kim et al., Archives of Pharmacal Res. 24(1):1-15, February 2001; and Anwer et al., Critical Reviews in Therapeutic Drug Carrier Systems 17(4):377-424, 2000. Genes encoding proteins that modulate cell cycle include the INK4 family of genes (U.S. Pat. No. 5,889,169; U.S. Pat. No. 6,033,847), ARF-p19 (U.S. Pat. No. 5,723,313), p21^{WAF1/CIP1} and p27^{KIP1} (WO 9513375; WO 9835022), p27^{KIP1} (WO 9738091), p57^{KIP2} (U.S. Pat. No. 6,025,480), ATM/ATR (WO 99/04266), Gadd 45 (U.S. Pat. No. 5,858,679), Myt1 (U.S. Pat. No. 5,744,349), Weel (WO 9949061) smad 3 and smad 4 (U.S. Pat. No. 6,100,032),14-3-3σ (WO 9931240), GSK3β (Stambolic, V. and Woodgett, J. R., Biochem Journal 303: 701-704,1994), HDAC-1 (Furukawa, Y. et al., Cytogenet. Cell Genet. 73: 130-133,1996; Taunton, J. et al., Science 272: 408-411,1996), PTEN (WO 9902704), p53 (U.S. Pat. No. 5,532,220), p33^{ING1} (U.S. Pat. No. 5,986,078), Retinoblastoma (EPO 390530), and NF-1 (WO 9200387).

A wide variety of gene delivery vehicles may be utilized to deliver and express the proteins described herein, including for example, viral vectors such as retroviral vectors (e.g., U.S. Pat. Nos. 5,591,624, 5,716,832, 5,817,491, 5,856,185, 5,888,502, 6,013,517, and 6,133,029; as well as subclasses of retroviral vectors such as lentiviral vectors (e.g., PCT Publication Nos. WO 00/66759, WO 00/00600, WO 99/24465, WO 98/51810, WO 99/51754, WO 99/31251, WO 99/30742, and WO 99/15641)), alphavirus based vector systems (e.g., U.S. Pat. Nos. 5,789,245, 5,814,482, 5,843,723, and 6,015,686), adeno-associated virus-based system (e.g., U.S. Pat. Nos. 6,221,646, 6,180,613, 6,165,781,6,156,303,6,153,436, 6,093,570, 6,040,183, 5,989,540, 5,856,152, and 5,587,308) and adenovirus-based systems (e.g., U.S. Pat. Nos. 6,210,939, 6,210,922, 6,203,975, 6,194,191, 6,140,087, 6,113,913, 6,080,569, 6,063,622, 6,040,174, 6,033,908, 6,033,885, 6,020,191, 6,020,172, 5,994,128, and 5,994,106), herpesvirus based or “amplicon” systems (e.g., U.S. Pat. Nos. 5,928,913, 5,501,979, 5,830,727, 5,661,033, 4,996,152 and 5,965,441) and, “naked DNA” based systems (e.g., U.S. Pat. Nos. 5,580,859 and 5,910,488) (all of which are, as noted above, incorporated by reference in their entirety).

Within one aspect of the invention, ribozymes or antisense sequences (as well as gene therapy vehicles which can deliver such sequences) can be utilized as cell cycle inhibitors. One representative example of such inhibitors is disclosed in PCT Publication No. WO 00/32765 (which, as noted above, is incorporated by reference in its entirety).

5. Cyclin Dependent Protein Kinase Inhibitors

In another embodiment, the pharmacologically active compound is a cyclin dependent protein kinase inhibitor (e.g., R-roscovitine, CYC-101, CYC-103, CYC-400, MX-7065, alvocidib (4H-1-Benzopyran-4-one, 2-(2-chlorophenyl)-5,7-dihydroxy-8-(3-hydroxy-1-methyl-4-piperidinyl)-, cis-(−)-[CAS]), SU-9516, AG-12275, PD-0166285, CGP-79807, fascaplysin, GW-8510 (Benzenesulfonamide, 4-(((Z)-(6,7-dihydro-7-oxo-8H-pyrrolo(2,3-g]benzothiazol-8-ylidene)methyl]amino]-N-(3-hydroxy-2,2-dimethylpropyl)-[CAS]), GW-491619, Indirubin 3′ monoxime, GW8510) or an analogue or derivative thereof.

6. EGF (Epidermal Growth Factor) Receptor Kinase Inhibitors

In another embodiment, the pharmacologically active compound is an EGF (epidermal growth factor) kinase inhibitor (e.g., erlotinib (4-Quinazolinamine, N-(3-ethynylphenyl)-6,7-bis(2-methoxyethoxy)-, monohydrochloride [CAS]), Viatris, erbstatin, BIBX-1382, gefitinib (4-Quinazolinamine, N-(3-chloro-4-fluorophenyl)-7-methoxy-6-(3-(4-morpholinyl)propoxy) [CAS])) or an analogue or derivative thereof.

7. Elastase Inhibitors

In another embodiment, the pharmacologically active compound is an elastase inhibitor (e.g., ONO-6818, sivelestat sodium hydrate (Glycine, N-(2-(((4-(2,2-dimethyl-1-oxopropoxy)phenyl]sulfonyl]amino]benzoyl]-[CAS]), erdosteine (Acetic acid, ((2-oxo-2-((tetrahydro-2-oxo-3-thienyl)amino]ethyl]thio]-[CAS]), MDL-100948a, MDL-104238 (N-(4-(4-morpholinylcarbonyl)benzoyl]-L-valyl-N′-(3,3,4,4,4-pentafluoro-1-(1-methylethyl)-2-oxobutyl]-L-2-azetamide), MDL-27324 (L-Prolinamide, N-((5-(dimethylamino)-1-naphthalenyl]sulfonyl]-L-alanyl-L-alanyl-N-(3,3,3-trifluoro-1-(1-methylethyl)-2-oxopropyl]-, (S)-[CAS]), SR-26831 (Thieno[3,2-c]pyrid inium, 5-((2-chlorophenyl)methyl]-2-(2,2-dimethyl-1-oxopropoxy)-4,5,6,7-tetrahydro-5-hydroxy-[CAS]), Win-68794, Win-63110, SSR-69071 (2-(9(2-Piperidinoethoxy)-4-oxo-4H-pyrido[1,2-a]pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (NAlpha-(1-adamantanesulphonyl)-N-(4-carboxybenzoyl)-L-lysyl-alanyl-L-valinal), R-665, FCE-28204, ((6R,7R)-2-(Benzoyloxy)-7-methoxy-3-methyl-4-pivaloyl-3-cephem 1,1-dioxide), 1,2-Benzisothiazol-3(2H)-one, 2-(2,4-dinitrophenyl)-, 1,1-dioxide [CAS], L-658758 (L-Proline, 1-((3-((acetyloxy)methyl]-7-methoxy-8-oxo-5-thia-1-azabicyclo(4.2.0]oct-2-en-2-yl]carbonyl]-, S,S-dioxide, (6R-cis)-[CAS]), L-659286 (Pyrrolidine, 1-((7-methoxy-8-oxo-3-(((1,2,5,6-tetrahydro-2-methyl-5,6-dioxo-1,2,4-triazin-3-yl)thio]methyl]-5-thia-1-azabicyclo(4.2.0]oct-2-en-2-yl]carbonyl]-, S,S-dioxide, (6R-cis)-[CAS]), L-680833 (Benzeneacetic acid, 4-((3,3-diethyl-1-(((1-(4-methylphenyl)butyl]amino]carbonyl]-4-oxo-2-azetidinyl]oxy]-, (S—(R*,S*)]-[CAS])) or an analogue or derivative thereof.

8. Factor Xa Inhibitors

In another embodiment, the pharmacologically active compound is a factor Xa inhibitor (e.g., CY-222, fondaparinux sodium (Alpha-D-Glucopyranoside, methyl 0-2-deoxy-6-O-sulfo-2-(sulfoamino)-Alpha-D-glucopyranosyl-(1-4)—O-1-D-glucopyranuronosyl-(1-4)—O-2-deoxy-3,6-di-O-sulfo-2-(sulfoamino)-Alpha-D-glucopyranosyl-(1-4)—O-2-O-sulfo-Alpha-L-idopyranuronosyl-(1-4)-2-deoxy-2-(sulfoamino)-, 6-(hydrogen sulfate) [CAS]), danaparoid sodium) or an analogue or derivative thereof.

9. Farnesyltransferase Inhibitors

In another embodiment, the pharmacologically active compound is a farnesyltransferase inhibitor (e.g., dichlorobenzoprim (2,4-diamino-5-(4-(3,4-dichlorobenzylamino)-3-nitrophenyl]-6-ethylpyrimidine), B-581, B-956 (N-(8(R)-Amino-2(S)-benzyl-5(S)-isopropyl-9-sulfanyl-3(Z),6(E)-nonadienoyl]-L-methionine), OSI-754, perillyl alcohol (1-Cyclohexene-1-methanol, 4-(1-methylethenyl)-[CAS], RPR-114334, lonafarnib (1-Piperidinecarboxamide, 4-(2-(4-((11 R)-3,10-dibromo-8-chloro-6,11-dihydro-5H-benzo(5,6]cyclohepta[1,2-b]pyridin-11-yl]-1-piperidinyl]-2-oxoethyl]-[CAS]), Sch-48755, Sch-226374, (7,8-Dichloro-5H-dibenzo(b,e](1,4]diazepin-11-yl)-pyrid in-3-ylmethylamine, J-104126, L-639749, L-731734 (Pentanamide, 2-((2-((2-amino-3-mercaptopropyl)amino]-3-methylpentyl]amino]-3-methyl-N-(tetrahydro-2-oxo-3-furanyl)-, (3S-(3R*(2R*(2R*(S*),3S*],3R*]]]-[CAS]), L-744832 (Butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))-[CAS]), L-745631 (1-piperazinepropanethiol, β-amino-2-(2-methoxyethyl)-4-(1-naphthalenylcarbonyl)-, (βR,2S)-[CAS]), N-acetyl-N-naphthylmethyl-2(S)-((1-(4-cyanobenzyl)-1H-imidazol-5-yl)acetyl]amino-3(S)-methylpentamine, (2Alpha)-2-hydroxy-24,25-dihydroxylanost-8-en-3-one, BMS-316810, UCF-1-C (2,4-Decadienamide, N-(5-hydroxy-5-(7-((2-hydroxy-5-oxo-1-cyclopenten-1-yl)amino-oxo-1,3,5-heptatrienyl)-2-oxo-7-oxabicyclo(4.1.0)hept-3-en-3-yl)-2,4,6-trimethyl-, (1S-(1 Alpha,3(2E,4E,6S*),5Alpha,5(1E,3E,5E),6Alpha))-[CAS]), UCF-116-B) or an analogue or derivative thereof.

10. Fibrinogen Antagonists

In another embodiment, the pharmacologically active compound is a fibrinogen antagonist (e.g., 2(S)-((p-Toluenesulfonyl)amino]-3-(((5,6,7,8,-tetrahydro-4-oxo-5-(2-(piperidin-4-yl)ethyl]-4H-pyrazolo-(1,5-a](1,4]diazepin-2-yl]carbonyl]-amino]propionic acid, streptokinase (Kinase (enzyme-activating), strepto-[CAS]), urokinase (Kinase (enzyme-activating), uro-[CAS]), plasminogen activator, pamiteplase, monteplase, heberkinase, anistreplase, alteplase, pro-urokinase, picotamide (1,3-Benzenedicarboxamide, 4-methoxy-N,N′-bis(3-pyridinylmethyl)-[CAS])) or an analogue or derivative thereof.

11. Guanylate Cyclase Stimulants

In another embodiment, the pharmacologically active compound is a guanylate cyclase stimulant (e.g., isosorbide-5-mononitrate (D-Glucitol, 1,4:3,6-dianhydro-, 5-nitrate [CAS])) or an analogue or derivative thereof.

12. Heat Shock Protein 90 Antagonists

In another embodiment, the pharmacologically active compound is a heat shock protein 90 antagonist (e.g., geldanamycin; NSC-33050 (17-Allylaminogeldanamycin), rifabutin (Rifamycin XIV, 1′,4-didehydro-1-deoxy-1,4-dihydro-5′-(2-methylpropyl)-1-oxo-[CAS]), 17AAG) or an analogue or derivative thereof.

13. HMGCOA Reductase Inhibitors

In another embodiment, the pharmacologically active compound is an HMGCoA reductase inhibitor (e.g., BCP-671, BB-476, fluvastatin (6-Heptenoic acid, 7-(3-(4-fluorophenyl)-1-(1-methylethyl)-1H-indol-2-yl]-3,5-dihydroxy-, monosodium salt, (R*,S*-(E)]-(±)-[CAS]), dalvastatin (2H-Pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4Alpha,6β(E))-(+/−)-[CAS]), glenvastatin (2H-Pyran-2-one, 6-(2-(4-(4-fluorophenyl)-2-(1-methylethyl)-6-phenyl-3-pyridinyl]ethenyl]tetrahydro-4-hydroxy-, (4R-(4Alpha,6β(E)]]-[CAS]), S-2468, N-(1-oxododecyl)-4Alpha,10-dimethyl-8-aza-trans-decal-3β-ol, atorvastatin calcium (1H-Pyrrole-1-heptanoic acid, 2-(4-fluorophenyl)-β,delta-dihydroxy-5-(1-methylethyl)-3-phenyl-4-((phenylamino)carbonyl]-, calcium salt (R—(R*,R*)]-[CAS]), CP-83101 (6,8-Nonadienoic acid, 3,5-dihydroxy-9,9-diphenyl-, methyl ester, (R*,S*-(E)]-(+/−)-[CAS]), pravastatin (1-Naphthaleneheptanoic acid, 1,2,6,7,8,8a-hexahydro-β,delta,6-trihydroxy-2-methyl-8-(2-methyl-1-oxobutoxy)-, monosodium salt, (1S-(1Alpha(βS*,deltaS*),2Alpha,6Alpha,8β(R*),8aAlpha]]-[CAS]), U-20685, pitavastatin (6-Heptenoic acid, 7-(2-cyclopropyl-4-(4-fluorophenyl)-3-quinolinyl]-3,5-dihydroxy-, calcium salt (2:1), (S—(R*,S*-(E)]]-[CAS]), N-((1-methylpropyl)carbonyl)-8-(2-(tetrahydro-4-hydroxy-6-oxo-2H-pyran-2-yl)ethyl]-perhydro-isoquinoline, dihydromevinolin (Butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl]-1-naphthalenyl ester(1 Alpha(R*),3Alpha,4aAlpha,7β,8β(2S*,4S*),8aβ]]-[CAS]), HBS-107, dihydromevinolin (Butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl]-1-naphthalenyl ester(1Alpha(R*),3Alpha,4aAlpha,7β,β8(2S*,4S*),8aβ]]-[CAS]), L-669262 (Butanoic acid, 2,2-dimethyl-, 1,2,6,7,8,8a-hexahydro-3,7-dimethyl-6-oxo-8-(2-(tetrahydro-4-hydroxy-6-oxo-2H-pyran-2-yl)ethyl]-1-naphthalenyl(1S-(1Alpha,7β,8β(2S*,4S*),8a]]]-[CAS]), simvastatin (Butanoic acid, 2,2-dimethyl-,1,2,3,7,8,8a-hexahydro-3,7-dimethyl-8-(2-(tetrahydro-4-hydroxy-6-oxo-2H-pyran-2-yl)ethyl]-1-naphthalenyl ester, (1S-(1Alpha,3Alpha,7β,8β(2S*,4S*),8aβ]]-[CAS]), rosuvastatin calcium (6-Heptenoic acid, 7-(4-(4-fluorophenyl)-6-(1-methylethyl)-2-(methyl(methylsulfonyl)amino)-5-pyrimdinyl)-3,5-dihydroxy-calcium salt (2:1) (S—(R*, S*-(E))) [CAS]), meglutol (2-hydroxy-2-methyl-1,3-propandicarboxylic acid), lovastatin (Butanoic acid, 2-methyl-, 1,2,3,7,8,8a-hexahydro-3,7-dimethyl-8-(2-(tetrahydro-4-hydroxy-6-oxo-2H-pyran-2-yl)ethyl]-1-naphthalenyl ester, (1S-(1.alpha.(R*),3Alpha,7β,β8(2S*,4S*),8aβ]]-[CAS])) or an analogue or derivative thereof.

14. Hydroorotate Dehydrogenase Inhibitors

In another embodiment, the pharmacologically active compound is a hydroorotate dehydrogenase inhibitor (e.g., leflunomide (4-Isoxazolecarboxamide, 5-methyl-N-(4-(trifluoromethyl)phenyl]-[CAS]), laflunimus (2-Propenamide, 2-cyano-3-cyclopropyl-3-hydroxy-N-(3-methyl-4(trifluoromethyl)phenyl)-, (Z)-[CAS])) or an analogue or derivative thereof.

15. IKK2 Inhibitors

In another embodiment, the pharmacologically active compound is an IKK2 inhibitor (e.g., MLN-120B, SPC-839) or an analogue or derivative thereof.

16. IL-1, ICE & IRAK Antagonists

In another embodiment, the pharmacologically active compound is an IL-1, ICE ((aryl)acyloxymethyl ketone) and IRAK antagonist (e.g., VX-765 (Vertex Pharmaceuticals Inc., Cambridge, Mass.), VX-740 (Vertex Pharmaceuticals Inc.), E-5090 (2-propenoic acid, 3-(5-ethyl-4-hydroxy-3-methoxy-1-naphthalenyl)-2-methyl-, (Z)-[CAS]), CH-164, CH-172, CH-490, AMG-719, iguratimod (N-(3-(Formylamino)-4-oxo-6-phenoxy-4H-chromen-7-yl] methanesulfonamide), AV94-88, pralnacasan (6H-Pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-[CAS]), (2S-cis)-5-(Benzyloxycarbonylamino-1,2,4,5,6,7-hexahydro-4-(oxoazepino(3,2,1-hi]indole-2-carbonyl)-amino]-4-oxobutanoic acid, AVE-9488, Esonarimod (Benzenebutanoic acid, Alpha-((acetylthio)methyl]-4-methyl-Gamma-oxo-[CAS], Taisho Pharmaceutical Co., Ltd., Japan), pralnacasan (6H-Pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-[CAS]), tranexamic acid (Cyclohexanecarboxylic acid, 4-(aminomethyl)-, trans-[CAS]), Win-72052, Romazarit (Ro-31-3948) (Propanoic acid, 2-((2-(4-chlorophenyl)-4-methyl-5-oxazolyl]methoxy]-2-methyl-[CAS]), PD-163594, SDZ-224-015 (L-Alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-[CAS]), L-709049 (L-Alaninamide, N-acetyl-L-tyrosyl-L-valyl-N-(2-carboxy-1-formylethyl)-, (S)-[CAS]), TA-383 (1H-Imidazole, 2-(4-chlorophenyl)-4,5-dihydro-4,5-diphenyl-, monohydrochloride, cis-[CAS]), EI-1507-1 (6a,12a-Epoxybenz(a]anthracen-1,12(2H,7H)-dione, 3,4-dihydro-3,7-dihydroxy-8-methoxy-3-methyl-[CAS]), Ethyl 4-(3,4-dimethoxyphenyl)-6,7-dimethoxy-2-(1,2,4-triazol-1-yl methyl)quinoline-3-carboxylate, EI-1941-1,

TJ-114, anakinra (Interleukin 1 receptor antagonist (human isoform x reduced), N2-L-methionyl-[CAS])) or an analogue or derivative thereof.

17. IL-4 Agonists

In another embodiment, the pharmacologically active compound is an IL-4 agonist (e.g., glatiramir acetate (L-Glutamic acid, polymer with L-alanine, L-lysine and L-tyrosine, acetate (salt) [CAS])) or an analogue or derivative thereof.

18. Immunomodulatory Agents

In another embodiment, the pharmacologically active compound is an immunomodulatory agent (e.g., Biolimus, leflunamide, ABT-578, methylsulfamic acid 3-(2-methoxyphenoxy)-2-(((methylamino)sulfonyl]oxy]propyl ester, sirolimus, CCl-779 (Rapamycin 42-(3-hydroxy-2-(hydroxymethyl)-2-methylpropanoate) [CAS]), LF-15-0195, N PC15669 (L-Leucine, N-(((2,7-dimethyl-9H-fluoren-9-yl)methoxy]carbonyl]-[CAS]), NPC-15670 (L-Leucine, N-(((4,5-dimethyl-9H-fluoren-9-yl)methoxy]carbonyl]-[CAS]), NPC-16570 (4-(2-(Fluoren-9-yl)ethyloxy-carbonyl]aminobenzoic acid), sufosfamide (Ethanol, 2-((3-(2-chloroethyl)tetrahydro-2H-1,3,2-oxazaphosphorin-2-yl]amino]-, methanesulfonate (ester), P-oxide [CAS]), tresperimus (2-(N-(4-(3-Aminopropylamino)butyl]carbamoyloxy]-N-(6-guanidinohexyl)acetamide), 4-(2-(Fluoren-9-yl)ethoxycarbonylamino]-benzo-hydroxamic acid, laquinimod, PBI-1411, azathioprine (6-((1-Methyl-4-nitro-1H-imidazol-5-yl)thiol-1H-purine), PBI0032, beclometasone, MDL-28842 (9H-Purin-6-amine, 9-(5-deoxy-5-fluoro-β-D-threo-pent-4-enofuranosyl)-, (Z)-[CAS]), FK-788, AVE-1726, ZK-90695, ZK-90695, Ro-54864, didemnin-B, Illinois (Didemnin A, N-(1-(2-hydroxy-1-oxopropyl)-L-prolyl]-, (S)-[CAS]), SDZ-62-826 (Ethanaminium, 2-((hydroxy((1-((octadecyloxy)carbonyl]-3-piperidinyl]methoxy]phosphinyl]oxy]-N,N, N-trimethyl-, inner salt [CAS]), argyrin B ((4S,7S,13R,22R)-13-Ethyl-4-(1H-indol-3-ylmethyl)-7-(4-methoxy-1H-indol-3-ylmethyl)18,22-dimethyl-16-methyl-ene-24-thia-3,6,9,12,15,18,21,26-octaazabicyclo(21.2.1]-hexacosa-1 (25),23(26)-diene-2,5,8,11,14,17,20-heptaone [CAS]), everolimus (Rapamycin, 42-O-(2-hydroxyethyl)-[CAS]), SAR-943, L-687795, 6-((4-Chlorophenyl)sulfinyl]-2,3-dihydro-2-(4-methoxy-phenyl)-5-methyl-3-oxo-4-pyridazinecarbonitrile, 91 Y78 (1H-Imidazo[4,5-c]pyridin-4-amine, 1-β-D-ribofuranosyl-[CAS]), auranofin (Gold, (1-thio-1-D-glucopyranose 2,3,4,6-tetraacetato-S)(triethylphosphine)-[CAS]), 27-O-Demethylrapamycin, tipredane (Androsta-1,4-dien-3-one, 17-(ethylthio)-9-fluoro-11-hydroxy-17-(methylthio)-, (11β,17Alpha)-[CAS]), AI-402, LY-178002 (4-Thiazolidinone, 5-((3,5-bis(1,1-dimethylethyl)-4-hydroxyphenyl]methylene]-[CAS]), SM-8849 (2-Thiazolamine, 4-(1-(2-fluoro(1,1′-biphenyl]-4-yl)ethyl]-N-methyl-[CAS]), piceatannol, resveratrol, triamcinolone acetonide (Pregna-1,4-diene-3,20-dione, 9-fluoro-11,21-dihydroxy-16,17-((1-methylethylidene)bis(oxy)]-, (11β,16Alpha)-[CAS]), ciclosporin (Cyclosporin A-[CAS]), tacrolimus (15,19-Epoxy-3H-pyrido(2,1-c)(1,4)oxaazacyclotricosine-1,7,20,21 (4H,23H)-tetrone, 5,6,8,11,12,13,14,15,16,17,18,19,24,25,26,26a-hexadecahydro-5,19-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1 S*,3S*,4S*)),4S*,5R*,8S*,9E,12R*,14R*,15S*,16R*,18S*,19S*,26aR*))-[CAS]), gusperimus (Heptanamide, 7-((aminoiminomethyl)amino]-N-(2-((4-((3-aminopropyl)amino]butyl]amino]-1-hydroxy-2-oxoethyl]-, (+/−)-[CAS]), tixocortol pivalate (Pregn-4-ene-3,20-dione, 21-((2,2-dimethyl-1-oxopropyl)thiol-11,17-dihydroxy-, (11β)-[CAS]), alefacept (1-92 LFA-3 (Antigen) (human) fusion protein with immunoglobulin G1 (human hinge-CH2—CH3 Gamma1-chain), dimmer), halobetasol propionate (Pregna-1,4-diene-3,20-dione, 21-chloro-6,9-difluoro-11-hydroxy-16-methyl-17-(1-oxopropoxy)-, (6Alpha,11β,16β)-[CAS]), iloprost trometamol (Pentanoic acid, 5-(hexahydro-5-hydroxy-4-(3-hydroxy-4-methyl-1-octen-6-ynyl)-2(1H)-pentalenylidene]-[CAS]), beraprost (1H-Cyclopenta(b]benzofuran-5-butanoic acid, 2,3,3a,8b-tetrahydro-2-hydroxy-1-(3-hydroxy-4-methyl-1-octen-6-ynyl)-[CAS]), rimexolone (Androsta-1,4-dien-3-one, 1′-hydroxy-16,17-dimethyl-17-(1-oxopropyl)-, (11β,16Alpha,17β)-[CAS]), dexamethasone (Pregna-1,4-diene-3,20-dione,9-fluoro-11,17,21-trihydroxy-16-methyl-, (11β,16Alpha)-[CAS]), sulindac (cis-5-fluoro-2-methyl-1-((p-methylsulfinyl)benzylidene]indene-3-acetic acid), proglumetacin (1H-lndole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (+/−)-[CAS]), alclometasone dipropionate (Pregna-1,4-diene-3,20-dione, 7-chloro-11-hydroxy-16-methyl-17,21-bis(1-oxopropoxy)-, (7Alpha, 11β,16Alpha)-[CAS]), pimecrolimus (15,19-Epoxy-3H-pyrido(2,1-c)(1,4)oxaazacyclotricosine-1,7,20,21 (4H,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*,15S*,16R*,18S*,19S*,26aR*)) -[CAS]), hydrocortisone-17-butyrate (Pregn-4-ene-3,20-dione, 11,21-dihydroxy-17-(1-oxobutoxy)-, (11β)-[CAS]), mitoxantrone (9,10-Anthracenedione, 1,4-dihydroxy-5,8-bis((2-((2-hydroxyethyl)amino]ethyl]amino]-[CAS]), mizoribine (1H-Imidazole-4-carboxamide, 5-hydroxy-1-1-D-ribofuranosyl-[CAS]), prednicarbate (Pregna-1,4-diene-3,20-dione, 17-((ethoxycarbonyl)oxy]-11-hydroxy-21-(1-oxopropoxy)-, (11β)-[CAS]), Iobenzarit (Benzoic acid, 2-((2-carboxyphenyl)amino]-4-chloro-[CAS]), glucametacin (D-Glucose, 2-(((1-(4-chlorobenzoyl)-5-methoxy-2-methyl-1H-indol-3-yl]acetyl]amino]-2-deoxy-[CAS]), fluocortolone monohydrate ((6Alpha)-fluoro-16Alpha-methylpregna-1,4-dien-11β,21-diol-3,20-dione), fluocortin butyl (Pregna-1,4-dien-21-oic acid, 6-fluoro-11-hydroxy-16-methyl-3,20-dioxo-, butyl ester, (6Alpha,11β,16Alpha)-[CAS]), difluprednate (Pregna-1,4-diene-3,20-dione, 21-(acetyloxy)-6,9-difluoro-11-hydroxy-17-(1-oxobutoxy)-, (6Alpha,11β)-[CAS]), diflorasone diacetate (Pregna-1,4-diene-3,20-dione, 17,21-bis(acetyloxy)-6,9-difluoro-11-hydroxy-16-methyl-, (6Alpha,11β,16β)-[CAS]), dexamethasone valerate (Pregna-1,4-diene-3,20-dione, 9-fluoro-11,21-dihydroxy-16-methyl-17-((1-oxopentyl)oxy]-, (11β,16Alpha)-[CAS]), methylprednisolone, deprodone propionate (Pregna-1,4-diene-3,20-dione, 11-hydroxy-17-(1-oxopropoxy)-, (11.beta.)-[CAS]), bucillamine (L-Cysteine, N-(2-mercapto-2-methyl-1-oxopropyl)-[CAS]), amcinonide (Benzeneacetic acid, 2-amino-3-benzoyl-, monosodium salt, monohydrate [CAS]), acemetacin (1H-Indole-3-acetic acid, 1-(4-chlorobenzoyl)-5-methoxy-2-methyl-, carboxymethyl ester [CAS])) or an analogue or derivative thereof. Further analogues of rapamycin include tacrolimus and derivatives thereof (e.g., EP0184162B1 and U.S. Pat. No. 6,258,823) and everolimus and derivatives thereof (e.g., U.S. Pat. No. 5,665,772). Further representative examples of sirolimus analogues and derivatives include ABT-578 and others may be found in PCT Publication Nos. WO9710502, WO9641807, WO9635423, WO9603430, WO9600282, WO9516691, WO9515328, WO9507468, WO9504738, WO9504060, WO9425022, WO9421644, WO9418207, WO9410843, WO9409010, WO9404540, WO9402485, WO9402137, WO9402136, WO9325533, WO9318043, WO9313663, WO9311130, WO9310122, WO9304680, WO9214737, and WO9205179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| Rapamune | NSC-226080 | | |
| Rapamycin | | | |
| Tacrolimus | FK506 | Fujusawa | See below |

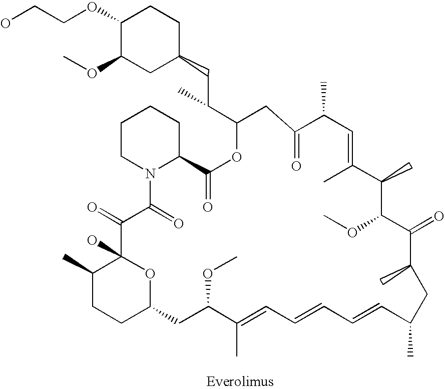

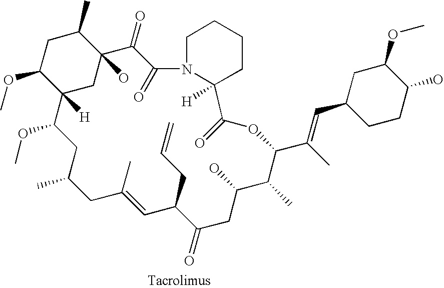

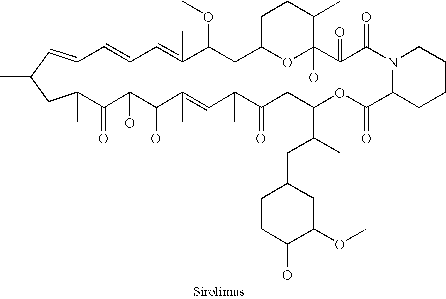

19. Inosine Monophosphate Dehydrogenase Inhibitors

In another embodiment, the pharmacologically active compound is an inosine monophosphate dehydrogenase inhibitor (e.g., Mycophenolate Mofetil (4-Hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-[CAS]), ribavirin (1H-1,2,4-Triazole-3-carboxamide, 1-β-D-ribofuranosyl-[CAS]), tiazofurin (4-Thiazolecarboxamide, 2-β-D-ribofuranosyl-[CAS]), viramidine, aminothiadiazole, thiophenfurin, tiazofurin) or an analogue or derivative thereof. Additional representative examples are included in U.S. Pat. Nos. 5,536,747; 5,807;876; 5,932,600; 6,054,472, 6,128,582; 6,344,465; 6,395,763; 6,399,773; 6,420,403; 6,479,628; 6,498,178; 6,514,979; 6,518291; 6541496; 6,596,747; 6,617,323; and 6,624,184, U.S. Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491 A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 0056331 A1, WO 00/73288A1, WO 01/00622A1, WO 01/66706A1, WO 01/79246A2, WO 01/81340A2, WO 01/85952A2, WO 02/16382A1, WO 02/18369A2, WO 02/51814A1, WO 02/57287A2, WO 02/57425A2, WO 02/60875A1, WO 02/60896A1, WO 02/60898A1, WO 02/68058A2, WO 03/20298A1, WO 03/37349A1, WO 03/39548A1, WO 03/45901A2, WO 03/47512A2, WO 03/53958A1, WO 03/55447A2, WO 03/59269A2, WO 03/63573A2, WO 03/87071A1, WO 90/01545A1, WO 97/40028A1, WO 97/41211A1, WO 98/40381A1, and WO 99/55663A1.

20. Leukotriene Inhibitors

In another embodiment, the pharmacologically active compound is a leukotreine inhibitor (e.g., DTI-0026, ONO-4057(Benzenepropanoic acid, 2-(4-carboxybutoxy)-6-((6-(4-methoxyphenyl)-5-hexenyl]oxy]-, (E)-[CAS]), ONO-LB-448, pirodomast 1,8-Naphthyridin-2(1H)-one, 4-hydroxy-1-phenyl-3-(1-pyrrolidinyl)-[CAS], Sch-40120 (Benzo(b](1,8]naphthyridin-5(7H)-one, 10-(3-chlorophenyl)-6,8,9,10-tetrahydro-[CAS]), L-656224 (4-Benzofuranol, 7-chloro-2-((4-methoxyphenyl)methyl]-3-methyl-5-propyl-[CAS]), MAFP (methyl arachidonyl fluorophosphonate), ontazolast (2-Benzoxazolamine, N-(2-cyclohexyl-1-(2-pyridinyl)ethyl]-5-methyl-, (S)-[CAS]), amelubant (Carbamic acid, ((4-((3-((4-(1-(4-hydroxyphenyl)-1-methylethyl)phenoxy)methyl)phenyl)methoxy)phenyl)iminomethyl)-ethyl ester [CAS]), SB-201993 (Benzoic acid, 3-((((6-((1E)-2-carboxyethenyl]-5-((8-(4-methoxyphenyl)octyl]oxy]-2-pyridinyl]methyl]thio]methyl]-[CAS]), LY-203647 (Ethanone, 1-(2-hydroxy-3-propyl-4-(4-(2-(4-(1H-tetrazol-5-yl)butyl]-2H-tetrazol-5-yl]butoxy]phenyl]-[CAS]), LY-210073, LY-223982 (Benzenepropanoic acid, 5-(3-carboxybenzoyl)-2-((6-(4-methoxyphenyl)-5-hexenyl]oxy]-, (E)-[CAS]), LY-293111 (Benzoic acid, 2-(3-(3-((5-ethyl-4′-fluoro-2-hydroxy(1,1′-biphenyl]-4-yl)oxy]propoxy]-2-propylphenoxy]-[CAS]), SM-9064 (Pyrrolidine,1-(4,11-dihydroxy-13-(4-methoxyphenyl)-1-oxo-5,7,9-tridecatrienyl]-, (E,E,E)-[CAS]), T-0757 (2,6-Octadienamide, N-(4-hydroxy-3,5-dimethylphenyl)-3,7-dimethyl-, (2E)-[CAS])) or an analogue or derivative thereof.

21. MCP-1 Antagonists

In another embodiment, the pharmacologically active compound is a MCP-1 antagonist (e.g., nitronaproxen (2-Napthaleneacetic acid, 6-methoxy-Alpha-methyl 4-(nitrooxy)butyl ester (AlphaS)-[CAS]), Bindarit (2-(1-benzylindazol-3-ylmethoxy)-2-methylpropanoic acid), 1-alpha-25 dihydroxy vitamin D₃) or an analogue or derivative thereof.

22. MMP Inhibitors

In another embodiment, the pharmacologically active compound is a MMP inhibitor (e.g., D-9120, doxycycline (2-Naphthacenecarboxamide, 4-(dimethylamino)-1,4,4a,5,5a,6,11,12a-octahydro-3,5,10,12,12a-pentahydroxy-6-methyl-1,11-dioxo-(4S-(4Alpha,4aAlpha,5Alpha,5aAlpha,6Alpha,12aAlpha)]-[CAS]), BB-2827, BB-1101 (2S-allyl-N-1-hydroxy-3R-isobutyl-N-4-(1S-methylcarbamoyl-2-phenylethyl)-succinamide), BB-2983, solimastat (N′-(2,2-Dimethyl-1 (S)—(N-(2-pyridyl)carbamoyl]propyl]-N-4-hydroxy-2(R)-isobutyl-3(S)-methoxysuccinamide), BATIMASTAT (Butanediamide, N4-hydroxy-N-1-(2-(methylamino)-2-oxo-1-(phenylmethyl)ethyl]-2-(2-methylpropyl)-3-((2-thienylthio)methyl]-, (2R-(1(S*),2R*,3S*]]-[CAS], British Biotech, UK), CH-138, CH-5902, D-1927, D-5410, EF-13 (Gamma-linolenic acid lithium salt),CMT-3 (2-Naphthacenecarboxamide, 1,4,4a,5,5a,6,11,12a-octahydro-3,10,12,12a-tetrahydroxy-1,11-dioxo-, (4aS,5aR,12aS)-[CAS]), MARIMASTAT (N-(2,2-Dimethyl-1 (S)—(N-methylcarbamoyl)propyl]-N,3(S)-dihydroxy-2(R)-isobutylsuccinamide, British Biotech, UK), TIMP'S,ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-[CAS]), PS-508, CH-715, nimesulide (Methanesulfonamide, N-(4-nitro-2-phenoxyphenyl)-[CAS]), hexahydro-2-(2(R)-(1 (RS)-(hydroxycarbamoyl)-4-phenylbutyl]nonanoyl]-N-(2,2,6,6-etramethyl-4-piperidinyl)-3(S)-pyridazine carboxamide, Rs-113-080, Ro-1130830, Cipemastat (1-Piperidinebutanamide, β-(cyclopentylmethyl)-N-hydroxy-Gamma-oxo-Alpha-((3,4,4-trimethyl-2,5-dioxo-1-imidazolidinyl)methyl]-,(AlphaR,βR)-[CAS]), 5-(4′-biphenyl)-5-(N-(4-nitrophenyl)piperazinyl]barbituric acid, 6-methoxy-1,2,3,4-tetrahydro-norharman-1-carboxylic acid, Ro-31-4724 (L-Alanine, N-(2-(2-(hydroxyamino)-2-oxoethyl]-4-methyl-1-oxopentyl]-L-leucyl-, ethyl ester[CAS]), prinomastat (3-Thiomorpholinecarboxamide, N-hydroxy-2,2-dimethyl-4-((4-(4-pyridinyloxy) phenyl)sulfonyl)-, (3R)-[CAS]), AG-3433 (1H-Pyrrole-3-propanic acid,1-(4′-cyano(1,1′-biphenyl]-4-yl)-b-((((3S)-tetrahydro-4,4-dimethyl-2-oxo-3-furanyl]amino]carbonyl]-, phenylmethyl ester, (bS)-[CAS]), PNU-142769 (2H-lsoindole-2-butanamide, 1,3-dihydro-N-hydroxy-Alpha-((3S)-3-(2-methylpropyl)-2-oxo-1-(2-phenylethyl)-3-pyrrolidinyl]-1,3-dioxo-, (AlphaR)-[CAS]), (S)-1-(2-((((4,5-Dihydro-5-thioxo-1,3,4-thiadiazol-2-yl)amino]-carbonyl]amino]-1-oxo-3-(pentafluorophenyl)propyl]-4-(2-pyridinyl)piperazine, SU-5402 (1H-Pyrrole-3-propanoic acid, 2-((1,2-dihydro-2-oxo-3H-indol-3-ylidene)methyl]-4-methyl-[CAS]), SC-77964, PNU-171829, CGS-27023a, N-hydroxy-2(R)-((4-methoxybenzene-sulfonyl)(4-picolyl)amino]-2-(2-tetrahydrofuranyl)-acetamide, L-758354 ((1,1′-Biphenyl)-4-hexanoic acid, Alpha-butyl-Gamma-(((2,2-dimethyl-1-((methylamino)carbonyl)propyl)amino)carbonyl)-4′-fluoro-, (AlphaS-(AlphaR*,GammaS*(R*)))-[CAS]), GI-155704a, CPA-926 or an analogue or derivative thereof. Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; 6,087,359.

23. NF kappa B Inhibitors

In another embodiment, the pharmacologically active compound is a NF kappa B inhibitor (e.g., Celgene (SP100030, SP100207, SP100393), AVE-0545, Oxi-104 (Benzamide, 4-amino-3-chloro-N-(2-(diethylamino)ethyl)-[CAS]), dexlipotam, INDRA, R-flurbiprofen ((1,1′-Biphenyl]-4-acetic acid, 2-fluoro-Alpha-methyl), SP100030 (2-chloro-N-(3,5-di(trifluoromethyl)phenyl]-4-(trifluoromethyl)pyrimidine-5-carboxamide), AVE-0545, Viatris, AVE-0547, Bay 11-7082, Bay 11-7085,15 deoxy-prostaylandin J2, bortezomib (Boronic acid, ((1R)-3-methyl-1-(((2S)-1-oxo-3-phenyl-2-((pyrazinylcarbonyl)amino]propyl]amino]butyl]-[CAS]) or an analogue or derivative thereof.

24. NO Agonists

In another embodiment, the pharmacologically active compound is a NO antagonist (e.g., NCX-4016 (Benzoic acid, 2-(acetyloxy)-, 3-((nitrooxy)methyl)phenyl ester [CAS]), NCX-2216, L-arginine or an analogue or derivative thereof.

25. P38 MAP Kinase Inhibitors

In another embodiment, the pharmacologically active compound is a P38 MAP kinase inhibitor (e.g., VX-745 (Vertex Pharmaceuticals, Inc., Cambridge, Mass.), GW-2286, SK86002, CGP-52411, BIRB-798, SB220025, RO-320-1195, RWJ-67657, RWJ-68354, SCIO-469, SCIO-323, AMG-548, CMC-146, SD-31145, CC-8866, Ro-320-1195, Roche (3853, 4507, 6145, 8464, 0945, 6257, 3391, 3470, 1151634, 5274, 5161, 4194, 1195), BIX 983 (Boehringer Ingelheim), PD-98059 (4H-1-Benzopyran-4-one, 2-(2-amino-3-methoxyphenyl)-[CAS]), CGH-2466, doramapimod, SB-203580 (Pyridine, 4-[5-(4-fluorophenyl)-2-[4-(methylsulfinyl)phenyl]-1H-imidazol-4-yl]-[CAS]), SB-220025 ((5-(2-Amino-4-pyrimidinyl)-4-(4-fluorophenyl)-1-(4-piperid inyl)imidazole)), SB-281832, PD169316, SB202190 or an analogue or derivative thereof. Additional representative examples are included in U.S. Pat. Nos. 6,300,347; 6,316,464; 6,316,466; 6,376,527; 6,444,696; 6,479,507; 6,509,361; 6,579,874; and 6,630,485, U.S. Publication Nos. 2001/0044538A1; 2002/0013354A1; 2002/0049220A1; 2002/0103245A1; 2002/0151491 A1; 2002/0156114A1; 2003/0018051A1; 2003/0073832A1; 2003/0130257A1; 2003/0130273A1; 2003/0130319A1; 2003/0139388A1; 2003/0139462A1; 2003/0149031A1; 2003/0166647A1; and 2003/0181411A1; and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2, WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1.

26. Phosphodiesterase Inhibitors

In another embodiment, the pharmacologically active compound is a phosphodiesterase inhibitor (e.g., CDP-840 (Pyridine, 4-((2R)-2-(3-(cyclopentyloxy)-4-methoxyphenyl]-2-phenylethyl]-[CAS]), CH-3697, CT-2820, D-22888 (Imidazo[1,5-a]pyrido[3,2-e]pyrazin-6(5H)-one, 9-ethyl-2-methoxy-7-methyl-5-propyl-[CAS]), D-4418 (8-Methoxyquinoline-5-(N-(2,5-d ichloropyrid in-3-yl)]carboxamide), 1-(3-cyclopentyloxy-4-methoxyphenyl)-2-(2,6-d ichloro-4-pyridyl) ethanone oxime, D-4396, ONO-6126, CDC-998, CDC-801, V-11294A (3-(3-(Cyclopentyloxy)-4-methoxybenzyl]-6-(ethylamino)-8-isopropyl-3H-purine hydrochloride), S,S′-methylene-bis(2-(8-cyclopropyl-3-propyl-6-(4-pyridylmethylamino)-2-thio-3H-purine)) tetrahyrochloride, Rolipram (2-Pyrrolidinone, 4-(3-(cyclopentyloxy)-4-methoxyphenyl]-[CAS]), CP-293121, CP-353164 (5-(3-Cyclopentyloxy-4-methoxyphenyl)pyridine-2-carboxamide), oxagrelate (6-Phthalazinecarboxylic acid, 3,4-dihydro-1-(hydroxymethyl)-5,7-dimethyl-4-oxo-, ethyl ester [CAS]), PD-168787, ibudilast (1-Propanone, 2-methyl-1-(2-(1-methylethyl)pyrazolo[1,5-a]pyridin-3-yl]-[CAS]), oxagrelate (6-Phthalazinecarboxylic acid, 3,4-dihydro-1-(hydroxymethyl)-5,7-dimethyl-4-oxo-, ethyl ester [CAS]), griseolic acid (Alpha-L-talo-Oct-4-enofuranuronic acid, 1-(6-amino-9H-purin-9-yl)-3,6-anhydro-6-C-carboxy-1,5-dideoxy-[CAS]), KW-4490, KS-506, T-440, roflumilast (Benzamide, 3-(cyclopropylmethoxy)-N-(3,5-dichloro-4-pyridinyl)-4-(difluoromethoxy)-[CAS]), rolipram, milrinone, triflusinal (Benzoic acid, 2-(acetyloxy)-4-(trifluoromethyl)-[CAS]), anagrelide hydrochloride (Imidazo[2,1-b]quinazolin-2(3H)-one, 6,7-dichloro-1,5-dihydro-, monohydrochloride [CAS]), cilostazol (2(1H)-Quinolinone, 6-(4-(1-cyclohexyl-1H-tetrazol-5-yl)butoxy]-3,4-dihydro-[CAS]), propentofylline (1H-Purine-2,6-dione, 3,7-dihydro-3-methyl-1-(5-oxohexyl)-7-propyl-[CAS]), sildenafil citrate (piperazine, 1-((3-(4,7-dihydro-1-methyl-7-oxo-3-propyl-1H-pyrazolo(4,3-d)pyrimid in-5-yl)-4-ethoxyphenyl)sulfonyl)-4-methyl, 2-hydroxy-1,2,3-propanetricarboxylate-(1:1) [CAS]), tadalafil (Pyrazino(1′,2′:1,6)pyrido(3,4-b)indole1,4-dione, 6-(1,3-benzodioxol-5-yl)-2,3,6,7,12,12a-hexahydro-2-methyl-, (6R-trans) [CAS]), vardenafil (piperazine, 1-(3-(1,4-dihydro-5-methyl(−4-oxo-7-propylimidazo[5,1-f)(1,2,4)-triazin-2-yl)-4-ethoxyphenyl)sulfonyl)-4-ethyl-[CAS]), milrinone ((3,4′-Bipyridine]-5-carbonitrile, 1,6-dihydro-2-methyl-6-oxo-[CAS]), enoximone (2H-Imidazol-2-one, 1,3-dihydro-4-methyl-5-(4-(methylthio)benzoyl]-[CAS]), theophylline (1H-Purine-2,6-dione, 3,7-dihydro-1,3-dimethyl-[CAS]), ibudilast (1-Propanone, 2-methyl-1-(2-(1-methylethyl)pyrazolo[1,5-a]pyridin-3-yl]-[CAS]), aminophylline (1H-Purine-2,6-dione, 3,7-dihydro-1,3-dimethyl-, compd. with 1,2-ethanediamine (2:1)-[CAS]), acebrophylline (7H-Purine-7-acetic acid, 1,2,3,6-tetrahydro-1,3-dimethyl-2,6-dioxo-,compd. with trans-4-(((2-amino-3,5-dibromophenyl)methyl]amino]cyclohexanol (1:1) [CAS]), plafibride (Propanamide, 2-(4-chlorophenoxy)-2-methyl-N-(((4-morpholinylmethyl)amino]carbonyl]-[CAS]), loprinone hydrochloride (3-Pyridinecarbonitrile, 1,2-dihydro-5-imidazo[1,2-a]pyrid in-6-yl-6-methyl-2-oxo-, monohydrochloride-[CAS]), fosfosal (Benzoic acid, 2-(phosphonooxy)-[CAS]), amrinone ((3,4′-Bipyridin]-6(1H)-one, 5-amino-[CAS]) or an analogue or derivative thereof.

27. TGF Beta Inhibitors

In another embodiment, the pharmacologically active compound is a TGF beta Inhibitor (e.g., mannose-6-phosphate, LF-984, tamoxifen (Ethanamine, 2-(4-(1,2-diphenyl-1-butenyl)phenoxy]-N,N-dimethyl-, (Z)-[CAS]), tranilast) or an analogue or derivative thereof.

28. Thromboxane A2 Antagonists

In another embodiment, the pharmacologically active compound is a thromboxane A2 antagonist (e.g., CGS-22652 (3-Pyridineheptanoic acid, .gamma.-(4-(((4-chlorophenyl)sulfonyl]amino]butyl]-, (.+−.)-[CAS]), ozagrel (2-Propenoic acid, 3-(4-(1H-imidazol-1-ylmethyl)phenyl]-, (E)-[CAS]), argatroban (2-Piperidinecarboxylic acid, 1-(5-((aminoiminomethyl)amino]-1-oxo-2-(((1,2,3,4-tetrahydro-3-methyl-8-quinolinyl)sulfonyl]amino]pentyl]-4-methyl-[CAS]), ramatroban (9H-Carbazole-9-propanoic acid, 3-(((4-fluorophenyl)sulfonyl]amino]-1,2,3,4-tetrahydro-, (R)-[CAS]), torasemide (3-Pyridinesulfonamide, N-(((1-methylethyl)amino]carbonyl]-4-((3-methylphenyl)amino]-[CAS]), gamma linoleic acid ((Z,Z,Z)-6,9,12-Octadecatrienoic acid [CAS]), seratrodast (Benzeneheptanoic acid, zeta-(2,4,5-trimethyl-3,6-dioxo-1,4-cyclohexadien-1-yl)-, (+/−)-[CAS]) or an analogue or derivative thereof.

29. TNFa Antagonists/TACE Inhibitors

In another embodiment, the pharmacologically active compound is a TNFa Antagonist/TACE Inhibitor (e.g., Celgene (CC10037, CC-1±1049, CC-10004, CC10083), E-5531 (2-Deoxy-6-O-(2-deoxy-3-O-(3(R)-(5(Z)-dodecenoyloxy]-decyl]-6-O-methyl-2-(3-oxotetradecanamido)-4-O-phosphono-β-D-glucopyranosyl]-3-O-(3(R)-hydroxydecyl]-2-(3-oxotetradecanamido)-Alpha-D-glucopyranose-1-O-phosphate), AZD-4717, glycophosphopeptical, UR-12715 (Benzoic acid, 2-hydroxy-5-((4-(3-(4-(2-methyl-1H-imidazol[4,5-c]pyridin-1-yl]methyl]-1-piperidinyl]-3-oxo-1-phenyl-1-propenyl]phenyl}azo] (Z) [CAS]), PMS-601, AM-87, xyloadenosine (9H-Purin-6-amine, 9-β-D-xylofuranosyl-[CAS]), RDP-58, RDP-59, BB2275, benzydamine, E-3330 (Undecanoic acid, 2-((4,5-dimethoxy-2-methyl-3,6-dioxo-1,4-cyclohexadien-1-yl)methylene]-, (E)-[CAS]), N-(D, L-2-(hydroxyaminocarbonyl)methyl-4-methylpentanoyl]-L-3-(2′-naphthyl)alanyl-L-alanine, 2-aminoethyl amide, CP-564959, MLN-608, SPC-839, ENMD-0997, Sch-23863 ((2-(10,11-Dihydro-5-ethoxy-5H-dibenzo (a,d] cyclohepten-S-yl]-N,N-dimethyl-ethanamine), SH-636, PKF-241-466, PKF-242-484, TNF-484a, cilomilast (Cis-4-cyano-4-(3-(cyclopentyloxy)-4-methoxyphenyl]cyclohexane-1-carboxylic acid), GW-3333, GW-4459, BMS-561392, AM-87, cloricromene (Acetic acid, ((8-chloro-3-(2-(diethylamino)ethyl]-4-methyl-2-oxo-2H-1-benzopyran-7-yl]oxy]-, ethyl ester [CAS]), thalidomide (1H-Isoindole-1,3(2H)-dione, 2-(2,6-dioxo-3-piperidinyl)-[CAS]), vesnarinone (piperazine, 1-(3,4-dimethoxybenzoyl)-4-(1,2,3,4-tetrahydro-2-oxo-6-quinolinyl)-[CAS]), infliximab, lentinan, etanercept (1-235-Tumor necrosis factor receptor (human) fusion protein with 236-467-immunoglobulin G1 (human gamma1-chain Fc fragment) [CAS]), diacerein (2-Anthracenecarboxylic acid, 4,5-bis(acetyloxy)-9,10-dihydro-9,10-dioxo-[CAS]) or an analogue or derivative thereof.

30. Tyrosine Kinase Inhibitors

In another embodiment, the pharmacologically active compound is a tyrosine kinase inhibitor (e.g., SKI-606, ER-068224, SD-208, N-(6-Benzothiazolyl)-4-(2-(1-piperazinyl)pyrid-5-yl)-2-pyrimid ineamine, celastrol (24,25,26-Trinoroleana-1 (10),3,5,7-tetraen-29-oic acid, 3-hydroxy-9,13-dimethyl-2-oxo-, (9.beta.,13Alpha,14β,20Alpha)-[CAS]), CP-127374 (Geldanamycin, 17-demethoxy-17-(2-propenylamino)-[CAS]), CP-564959, PD-171026, CGP-52411 (H-1 soindole-1,3(2H)-dione, 4,5-bis(phenylamino)-[CAS]), CGP-53716 (Benzamide, N-(4-methyl-3-((4-(3-pyridinyl)-2-pyrimidinyl]amino]phenyl]-[CAS]), imatinib (4-((Methyl-1-piperazinyl)methyl)-N-(4-methyl-3-((4-(3-pyridinyl)-2-pyrimidinyl]amino]-phenyl]benzamide methanesulfonate), NVP-MK980-NX, KF-250706 (13-Chloro,5(R),6(S)-epoxy-14,16-dihydroxy-11-(hydroyimino)-3(R)-methyl-3,4,5,6,11,12-hexahydro-1H-2-benzoxacyclotetradecin-1-one), 5-(3-(3-methoxy-4-(2-((E)-2-phenylethenyl]-4-oxazolylmethoxy]phenyl]propyl]-3-(2-((E)-2-phenylethenyl]-4-oxazolylmethyl]-2,4-oxazolidinedione, genistein or an analogue or derivative thereof.

31. Vitronectin Inhibitors

In another embodiment, the pharmacologically active compound is a vitronectin inhibitor (e.g., O-(9,10-dimethoxy-1,2,3,4,5,6-hexahydro-4-((1,4,5,6-tetrahydro-2-pyrimidinyl)hydrazono]-8-benz(e)azulenyl]-N-((phenylmethoxy)carbonyl]-DL-homoserine 2,3-dihydroxypropyl ester, (2S)-Benzoylcarbonylamino-3-(2-((4S)-(3-(4,5-dihydro-1H-imidazol-2-ylamino)-propyl)-2,5-dioxo-imidazolidin-1-yl)-acetylamino]-propionate, Sch-221153, S-836, SC-68448 (R-((2-2-(((3-((aminoiminomethyl)amino]-phenyl]carbonyl]amino]acetyl]amino]-3,5-dichlorobenzenepropanoic acid), SD-7784, S-247) or an analogue or derivative thereof.

32. Fibroblast Growth Factor Inhibitors

In another embodiment, the pharmacologically active compound is a fibroblast growth factor inhibitor (e.g., CT-052923 ([(2H-benzo[d]1,3-dioxalan-5-methyl)amino][4-(6,7-dimethoxyquinazolin-4-yl)piperazinyl]methane-1-thione) or an analogue or derivative thereof.

33. Protein Kinase Inhibitors

In another embodiment, the pharmacologically active compound is a protein kinase inhibitor (e.g., KP-0201448, NPC15437 (Hexanamide, 2,6-d iamino-N-((1-(1-oxotridecyl)-2-piperidinyl]methyl]-[CAS]), fasudil (1H-1,4-Diazepine, hexahydro-1-(5-isoquinolinylsulfonyl)-[CAS]), midostaurin (Benzamide, N-(2,3,10,11,12,13-hexahydro-10-methoxy-9-methyl-1-oxo-9,13-epoxy-1H,9H-diindolo(1,2,3-gh:3′,2′,1′-Im]pyrrolo(3,4-j](1,7]benzodiazonin-11-yl)-N-methyl-, (9Alpha,10β,11β,13Alpha)-[CAS]), fasudil (1H-1,4-Diazepine, hexahydro-1-(5-isoquinolinylsulfonyl)-[CAS]) or an analogue or derivative thereof.

34. PDGF Receptor Kinase Inhibitors

In another embodiment, the pharmacologically active compound is a PDGF receptor kinase inhibitor (e.g., RPR-127963E) or an analogue or derivative thereof.

35. Endothelial Growth Factor Receptor Kinase Inhibitors

In another embodiment, the pharmacologically active compound is an endothelial growth factor receptor kinase inhibitor (e.g., CEP-7055, SU-0879 ((E)-3-(3,5-di-tert-Butyl-4-hydroxyphenyl)-2-(aminothiocarbonyl)acrylonitrile), BIBF-1000 or an analogue or derivative thereof.

36. Retinoic Acid Receptor Antagonists

In another embodiment, the pharmacologically active compound is a retinoic acid receptor antagonist (e.g., etarotene (Ro-15-1570) (Naphthalene, 6-(2-(4-(ethylsulfonyl)phenyl]-1-methylethenyl]-1,2,3,4-tetrahydro-1,1,4,4-tetramethyl-, (E)-[CAS]), (2E,4E)-3-Methyl-5-(2-((E)-2-(2,6,6-trimethyl-1-cyclohexen-1-yl)ethenyl)-1-cyclohexen-1-yl)-2,4-pentadienoic acid, tocoretinate (Retinoic acid, 3,4-dihydro-2,5,7,8-tetramethyl-2-(4,8,12-trimethyltridecyl)-2H-1-benzopyran-6-yl ester, (2R*(4R*,8R*)]-(O)-[CAS]), aliretinoin (Retinoic acid, cis-9, trans-13-[CAS]), bexarotene (Benzoic acid, 4-(1-(5,6,7,8-tetrahydro-3,5,5,8,8-pentamethyl-2-naphthalenyl)ethenyl)-[CAS]) or an analogue or derivative thereof.

37. Platelet Derived Growth Factor Receptor Kinase Inhibitors

In another embodiment, the pharmacologically active compound is a platelet derived growth factor receptor kinase inhibitor (e.g., leflunomide (4-lsoxazolecarboxamide, 5-methyl-N-(4-(trifluoromethyl)phenyl])-[CAS]) or an analogue or derivative thereof.

38. Fibronogin Antagonists

In another embodiment, the pharmacologically active compound is a fibrinogin antagonist (e.g., picotamide (1,3-Benzenedicarboxamide, 4-methoxy-N,N′-bis(3-pyridinylmethyl)-[CAS]) or an analogue or derivative thereof.

39. Antimycotic Agents

In another embodiment, the pharmacologically active compound is an antimycotic agent (e.g., miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (piperazine, 1-(4-((2-(2,4-dichlorophenyl)-2-(1H-1,2,4-triazol-1-ylmethyl)-1,3-dioxolan-4-yl]methoxy]phenyl]-4-(1-methylethyl)-, cis-[CAS]), isoconazole (1-(2-(2-6-dichlorobenzyloxy)-2-(2-,4-dichlorophenyl)ethyl]), griseofulvin (Spiro(benzofuran-2(3H),1′-(2]cyclohexane]-3,4′-dione, 7-chloro-2′,4,6-trimeth-oxy-6′methyl-, (1′S-trans)-[CAS]), bifonazole (1H-Imidazole, 1-((1,1′-biphenyl]-4-ylphenylmethyl)-[CAS]), econazole nitrate (1-(2-((4-chlorophenyl)methoxy]-2-(2,4-dichlorophenyl)ethyl]-1H-imidazole nitrate), croconazole (1H-Imidazole, 1-(1-(2-((3-chlorophenyl)methoxy]phenyl]ethenyl]-[CAS]), sertaconazole (1H-Imidazole, 1-(2-((7-chlorobenzo(b]thien-3-yl)methoxy]-2-(2,4-dichlorophenyl)ethyl]-[CAS]), omoconazole (1H-Imidazole, 1-(2-(2-(4-chlorophenoxy)ethoxy]-2-(2,4-dichlorophenyl)-1-methylethenyl]-, (Z)-[CAS]), flutrimazole (1H-1 midazole, 1-((2-fluorophenyl)(4-fluorophenyl)phenylmethyl]-[CAS]), fluconazole (1H-1,2,4-Triazole-1-ethanol, Alpha-(2,4-difluorophenyl)-Alpha-(1H-1,2,4-triazol-1-ylmethyl)-[CAS]), neticonazole (1H-Imidazole, 1-(2-(methylthio)-1-(2-(pentyloxy)phenyl]ethenyl]-, monohydrochloride, (E)-[CAS]), butoconazole (1H-Imidazole, 1-(4-(4-chlorophenyl)-2-((2,6-dichlorophenyl)thio]butyl]-, (+/−)-[CAS]), clotrimazole (1-((2-chlorophenyl)diphenylmethyl]-1H-imidazole) or an analogue or derivative thereof.

40. Bisphosphonates

In another embodiment, the pharmacologically active compound is a bisphosphonate (e.g., clodronate, alendronate, pamidronate, zoledronate, etidronate) or an analogue or derivative thereof.

41. Phospholipase A1 Inhibitors

In another embodiment, the pharmacologically active compound is a phospholipase A1 inhibitor (e.g., loteprednol etabonate (Androsta-1,4-diene-17-carboxylic acid, 17-((ethoxycarbonyl)oxy]-11-hydroxy-3-oxo-, chloromethyl ester, (11β,17Alpha)-[CAS] or an analogue or derivative thereof.

42. Histamine H1/H2/H3 Receptor Antagonists

In another embodiment, the pharmacologically active compound is a histamine H1/H2/H3 receptor antagonist (e.g., ranitidine (1,1-Ethenediamine, N-(2-(((5-((dimethylamino)methyl]-2-furanyl]methyl]thio]ethyl]-N′-methyl-2-nitro-[CAS]), niperotidine (N-(2-((5-((dimethylamino)methyl]furfuryl]thio]ethyl]-2-nitro-N′-piperonyl-1,1-ethenediamine), famotidine (Propanimidamide, 3-(((2-((aminoiminomethyl)amino]-4-thiazolyl]methyl]thiol-N-(aminosulfonyl)-[CAS]), roxitadine acetate HCl (Acetamide, 2-(acetyloxy)-N-(3-(3-(1-piperidinylmethyl)phenoxy]propyl]-, monohydrochloride [CAS]), lafutid ine (Acetamide, 2-((2-furanylmethyl)sulfinyl]-N-(4-((4-(1-piperidinylmethyl)-2-pyridinyl]oxy]-2-butenyl]-, (Z)-[CAS]), nizatadine (1,1-Ethenediamine, N-(2-(((2-((dimethylamino)methyl]-4-thiazolyl]methyl]thio]ethyl]-N′-methyl-2-nitro-[CAS]), ebrotidine (Benzenesulfonamide, N-(((2-(((2-((aminoiminomethyl)amino]-4-thiazoly]methyl]thio]ethyl]amino]methylene]-4-bromo-[CAS]), rupatadine (5H-Benzo(5,6]cyclohepta[1,2-b]pyridine, 8-chloro-6,11-dihydro-11-(1-((5-methyl-3-pyridinyl)methyl]-4-piperidinylidene]-, trihydrochloride-[CAS]), fexofenadine HCl (Benzeneacetic acid, 4-(1-hydroxy-4-(4(hydroxyd iphenylmethyl)-1-piperidinyl]butyl]-Alpha,Alpha-dimethyl-, hydrochloride [CAS]) or an analogue or derivative thereof.

43. Macrolide Antibiotics

In another embodiment, the pharmacologically active compound is a macrolide antibiotic (e.g., dirithromycin (Erythromycin, 9-deoxo-11-deoxy-9,11-(imino(2-(2-methoxyethoxy)ethylidene]oxy]-, (9S(R)]-[CAS]), flurithromycin ethylsuccinate (Erythromycin, 8-fluoro-mono(ethyl butanedioate) (ester)-[CAS]), erythromycin stinoprate (Erythromycin, 2′-propanoate, compd. with N-acetyl-L-cysteine (1:1) [CAS]), clarithromycin (Erythromycin, 6-O-methyl-[CAS]), azithromycin (9-deoxo-9a-aza-9a-methyl-9a-homoerythromycin-A), telithromycin (3-De((2,6-dideoxy-3-C-methyl-3-O-methyl-Alpha-L-ribo-hexopyranosyl)oxy)-11,12-dideoxy-6-O-methyl-3-oxo-12,11-(oxycarbonyl((4-(4-(3-pyridinyl)-1H-imidazol-1-yl)butyl)imino))-[CAS]), roxithromycin (Erythromycin, 9-(O-((2-methoxyethoxy)methyl]oxime] [CAS]), rokitamycin (Leucomycin V, 4B-butanoate 3B-propanoate [CAS]), RV-11 (erythromycin monopropionate mercaptosuccinate), midecamycin acetate (Leucomycin V, 3B,9-diacetate 3,4B-dipropanoate [CAS]), midecamycin (Leucomycin V, 3,4B-dipropanoate [CAS]), josamycin (Leucomycin V, 3-acetate 4B-(3-methylbutanoate) [CAS]) or an analogue or derivative thereof.

44. GPIIb IIIa Receptor Antagonists

In another embodiment, the pharmacologically active compound is an GPIIb IIIa receptor antagonist (e.g., tirofiban hydrochloride (L-Tyrosine, N-(butylsulfonyl)-O-(4-(4-piperid inyl)butyl]-, monohydrochloride-[CAS]), eptifibatide (L-Cysteinamide, N6-(aminoiminomethyl)-N-2-(3-mercapto-1-oxopropyl)-L-lysylglycyl-L-Alpha-aspartyl-L-tryptophyl-L-prolyl-, cyclic(1->6)-disulfide [CAS]) or an analogue or derivative thereof.

45. Endothelin Receptor Antagonists

In another embodiment, the pharmacologically active compound is an endothelin receptor antagonist (e.g., bosentan (Benzenesulfonamide, 4-(1,1-dimethylethyl)-N-(6-(2-hydroxyethoxy)-5-(2-methoxyphenoxy)(2,2′-bipyrimidin]-4-yl]-[CAS]) or an analogue or derivative thereof.

46. Peroxisome Proliferator-Activated Receptor Agonists

In another embodiment, the pharmacologically active compound is a peroxisome proliferators-activated receptor agonist (e.g., gemfibrozil (Pentanoic acid, 5-(2,5-dimethylphenoxy)-2,2-dimethyl-[CAS]), fenofibrate (Propanoic acid, 2-(4-(4-chlorobenzoyl)phenoxy]-2-methyl-, 1-methylethyl ester [CAS]), ciprofibrate (Propanoic acid, 2-(4-(2,2-d ichlorocyclopropyl)phenoxy]-2-methyl-[CAS]), rosiglitazone maleate (2,4-Thiazolidinedione, 5-((4-(2-(methyl-2-pyridinylamino)ethoxy)phenyl)methyl)-, (Z)-2-butenedioate (1:1) [CAS]), pioglitazone hydrochloride (2,4-Thiazolidinedione, 5-((4-(2-(5-ethyl-2-pyridinyl)ethoxy]phenyl]methyl]-, monohydrochloride (+/−)-[CAS]), etofylline clofibrate (Propanoic acid, 2-(4-chlorophenoxy)-2-methyl-, 2-(1,2,3,6-tetrahydro-1,3-dimethyl-2,6-dioxo-7H-purin-7-yl)ethyl ester [CAS]), etofibrate (3-Pyridinecarboxylic acid, 2-(2-(4-chlorophenoxy)-2-methyl-1-oxopropoxy]ethyl ester [CAS]), clinofibrate (Butanoic acid, 2,2′-(cyclohexylidenebis(4,1-phenyleneoxy)]bis(2-methyl-][CAS]), bezafibrate (Propanoic acid, 2-(4-(2-((4-chlorobenzoyl)amino]ethyl]phenoxy]-2-methyl-[CAS]), binifibrate (3-Pyridinecarboxylic acid, 2-(2-(4-chlorophenoxy)-2-methyl-1-oxopropoxy]-1,3-propanediyl ester [CAS]) or an analogue or derivative thereof.

47. Estrogen Receptor Agents

In another embodiment, the pharmacologically active compound is an estrogen receptor agent (e.g., estradiol, 17-β-estradio)l or an analogue or derivative thereof.

48. Somatostatin Analogues

In another embodiment, the pharmacologically active compound is somatostatin or a somatostatin analogue (e.g., angiopeptin, lanretide, octreotide) or an analogue or derivative thereof.

49. JNK (Jun Kinase) Inhibitors

In another embodiment, the pharmacologically active compound is a JNK Kinase inhibitor (e.g., Celgene (SP600125, SPC105, SPC23105), AS-602801 (Serono)) or an analogue or derivative thereof.

50. Melanocortin Analogues

In another embodiment, the pharmacologically active compound is a melanocortin analogue (e.g., HP228) or an analogue or derivative thereof).

51. RAF Kinase Inhibitors

In yet another embodiment, the pharmacologically active compound is a raf kinase inhibitor (e.g., BAY-43-9006 (N-(4-chloro-3-(trifluoromethyl)phenyl-N′-(4-(2-(N-methylcarbamoyl)-4-pyridyloxy)phenyl)urea) or analogue or derivative thereof.

52. Lysylhydroxylase Inhibitors

In another embodiment, the pharmacologically active compound is a lysylhydroxylase inhibitor (e.g., minoxidil), or an analogue or derivative thereof.

53. IKK 1/2 Inhibitors

In another embodiment, the pharmacologically active compound is an IKK 1/2 inhibitor (e.g., BMS-345541, SPC839), or an analogue or derivative thereof.

In addition to incorporation of a fibrosis-inhibiting agent into or onto the formulation, another biologically active agent can be incorporated into or onto the formulation, for example an anti-inflammatory (e.g., dexamethazone or asprin), antithrombotic agents (e.g., heparin, heparin complexes, hydrophobic heparin derivatives, aspirin, or dipyridamole), and/or an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

Optional Composition Properties and Packaging

In one aspect, the compositions of the present invention include one or more preservatives or bacteriostatic agents, present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the compositions of the present invention include one or more antioxidants, present in an effective amount. Examples of the antioxidant include sulfites, alpha-tocopherol and ascorbic acid.

In one aspect, the compositions of the present invention include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth.

In one aspect, the compounds and compositions of the present invention are sterile. Many pharmaceuticals are manufactured to be sterile and this criterion is defined by the USP XXII <121I>. The term “USP” refers to U.S. Pharmacopeia (see www.usp.org, Rockville, Md.). Sterilization in this embodiment may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E. I. DuPont De Nemours and Company, Wilmington, Del.).

In another aspect, the compositions of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, Teflon, silicone, and gray-butyl rubber. For parenterals, USP Types I to III glass and polyethylene are preferred.

Incorporation of Biologically Active Agents into the Compositions

Biologically active agents can be incorporated directly into the composition or they can be incorporated into a secondary carrier. For direct incorporation of the biologically active agent, the agent may or may not contain a nucleophilic group or groups that can react with the activated functional groups of the synthetic polymer of the composition. The biologically active agents can be incorporated as a solid with the activated polymer, be incorporated into an acidic buffer solution that can be used to solubilize the activated polymer, be incorporated into a basic solution that it then mixed with the activated polymer to increase the reaction time. In another embodiment, a combination of these methods could also be used to incorporate the biologically active agent into the composition. In another embodiment, the biologically active agent can be applied prior to, simultaneously or post-application of the activated polymer. The presence of the appropriate nucleophilic group(s) on the biologically active agent will allow the biologically active agent to be incorporated into the final composition via chemical bonds. A single biologically active agent may be directly incorporated into the composition or a combination of biologically active agents may be incorporated into the composition using any of the possible approaches described above.

For the incorporation of the biologically active agent into the composition via the use of a secondary carrier, the biologically active agent can be incorporated into the secondary carrier by covalent linking to the secondary carrier, physical entrapment, adsorption, electrostatic interactions, hydrophobic interactions, partitioning effects, precipitation in the secondary carrier or a combination of these interactions. This biologically active agent/secondary carrier composition can then be incorporated directly into the composition. The secondary carriers that can be used to incorporate these biologically active agents include particulates, microparticles, nanoparticles, nonocrystals, microspheres, nanospheres, liposomes, micelles, emulsions, microemulsions, dispersions, inclusion complexes, Non-ionic surfactant vesicles (NISV), niosomes, proniosomes, cochleates, immunostimulating complexes (ISCOMS) and association complexes. In one embodiment, the microparticles, nanoparticles or microspheres can be prepared using polymers and copolymers comprising one or more of the residue units of the monomers D-lactide, L-lactide, D,L-lactide, glycolide, ε-caprolactone, trimethylene carbonate, 1,4-dioxane-2-one or 1,5-dioxepan-2one. In another embodiment, the microparticles, nanoparticles or microspheres can be prepared using block copolymers of the for A-B, A-B-A or B-A-B where A is a poly(alkylene oxide) (e.g., poly(ethylene glycol), poly(propylene glycol), copolymers of ethylene oxide and propylene oxide, or mono-alkyl ethers thereof) and B is a degradable polyester, for example polymers and copolymers comprising one or more of the residue units of the monomers D-lactide, L-lactide, D,L-lactide, glycolide, ε-caprolactone, trimethylene carbonate, 1,4-dioxane-2-one or 1,5-dioxepan-2-one). Micelles can be prepared using small molecule surfactants (e.g., SDS) or polymeric compositions (e.g., PLURONICS F127, PLURONICS F68, block copolymers of the for A-B, A-B-A or B-A-B where A is a poly(alkylene oxide) (e.g., poly(ethylene glycol), poly(propylene glycol), copolymers of ethylene oxide and propylene oxide, or mono-alkyl ethers thereof) and be is a degradable polyester, for example polymers and copolymers comprising one or more of the residue units of the monomers D-lactide, L-lactide, D,L-lactide, glycolide, ε-caprolactone, trimethylene carbonate, 1,4-dioxane-2-one or 1,5-dioxepan-2-one). Albumin, alginate, gelatin, starch, collagen, chitosan, poly(anhydrides), poly(orthoesters), poly(phosphazines) can also be used to prepare these secondary carriers. Liposome compositions can include phosphatidyl choline, cholesterol, phosphatidyl ethanolamine as well as any of the commercially available lipids (for example, lipids available from Avanti Polar Lipids). Non-polymeric compounds such as sucrose derivatives (e.g., sucrose acetate isobutyrate, sucrose oleate), sterols such as cholesterol, stigmasterol, .β.-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate can also be used as part of the secondary carrier composition.

The biologically active agent/secondary carrier can be incorporated as a solid with the activated polymer, be incorporated into an acidic buffer solution that can be used to solubilize the activated polymer, be incorporated into a basic solution that it then mixed with the activated polymer to increase the reaction time. A combination of these methods could also be used to incorporate the biologically active agent/secondary carrier into the composition.

The biologically active agent/secondary carrier composition can contain groups that may or may not be able to react with the activated groups of the starting components. In one embodiment, the secondary carrier does not contain nucleophilic groups that can react with the starting polymer components, in which case the secondary carrier/biologically active agent is retained within the final composition through physical entrapment, hydrophobic, hydrogen bonding, Van der Waals interactions, electrostatic interactions or a combination of these interactive forces.

In another embodiment, the biologically active agent/secondary carrier composition may contain functional groups that can react with either the nucleophilic groups of the starting components. Under these circumstances, the biologically active agent/secondary carrier composition is retained in the final composition via covalent bonds. Other interactions such as physical entrapment, hydrophobic, hydrogen bonding, Van der Waals interactions, electrostatic interactions or a combination of these interactive forces may also contribute to the retention of the biologically active agent/secondary carrier in the final composition.

Compounds containing one or more of the following functional groups: —NH₂, —SH, —OH, —PH₂, —CO—NH—NH₂, —CO₂ N(COCH₂)₂, —CO₂H, —CHO, —CHOCH₂, —N═C═O, —SO₂ CH═CH₂, —N(COCH)), —S—S—(C₅H₄N), etc are compounds that can be incorporated into the secondary carriers thereby providing the secondary carriers with functional groups that are capable of reacting with the starting components of the crosslinked composition.

Examples of useful amino compounds that can be incorporated into the secondary carriers to provide functional groups on the secondary carrier include phosphatidyl ethanolamine lipids (for example, Avanti Polar Lipids, Inc Catalogue # 850757, 850756, 850759, 850801, 850758, 850802, 850804, 850806, 850697, 850699, 850700, 850702, 850745, 850705, 850402, 850706, 830756C, 830756P, 850715, 850725, 85T725, 850755, 850795, 850800, 850797, 870125, 870122, 870140, 870142, 856705, 856715, 846725), alkyl amines, aryl amines, cycloalkyl amines.

Examples of useful thiol compounds that can be incorporated into the secondary carriers to provide functional groups on the secondary carrier includes 1,2-Dipalmitoyl-sn-Glycero-3-Phosphothioethanol (Sodium Salt) (Avanti Polar Lipids, Catalogue # 870160), alkyl thiols, aryl thiols.

Use of the Compositions for Reduction of Surgical Adhesions

Adhesion formation, a complex process in which bodily tissues that are normally separate grow together, is most commonly seen to occur as a result of surgical trauma. Adhesions can occur following abdominal, pelvic, cardiac, spinal, tendon, cranial, peripheral nerve, nasal, ear or throat surgery. These post-operative adhesions occur in 60 to 90% of patients undergoing major gynacologic surgery and represent one of the most common causes of intestinal obstruction and infertility in the industrialized world. Other adhesion-treated complications include chronic pelvic pain, urethral obstruction and voiding dysfunction. Currently, preventative therapies, such inert surgical barriers made of hyaluronic acid or cellulose placed at the operative site at the time of surgery, are used to inhibit adhesion formation. In-situ crosslinking polymer formulations have been approved for use in cardiac (ADHIBIT from Cohesion Technologies, Palo Alto, Calif.) and abdominal and pelvic surgery (SPRAYGEL from Confluent Surgical, Inc., Boston, Mass.). Various modes of adhesion prevention have been examined, including (1) prevention of fibrin deposition, (2) reduction of local tissue inflammation and (3) removal of fibrin deposits. Fibrin deposition is prevented through the use of physical barriers that are either mechanical or comprised of viscous solutions. Although many investigators are utilizing adhesion prevention barriers, a number of technical difficulties exist. Inflammation is reduced by the administration of drugs such as corticosteroids and nonsteroidal anti-inflammatory drugs. However, the results from the use of these drugs in animal models have not been encouraging due to the extent of the inflammatory response and dose restriction due to systemic side effects. Finally, the removal of fibrin deposits has been investigated using proteolytic and fibrinolytic enzymes. A potential complication to the clinical use of these enzymes is the possibility for excessive bleeding.

Thus, within other aspects of the invention, methods are provided for treating and/or preventing adhesions by administering to the patient an activated polymer composition. This composition may also comprise a biologically active agent. The preferred biologically active agents to be used in this application are described above. Similarly the various methods for incorporating these biologically active agents into the composition are described above.

A wide variety of animal models may be utilized in order to assess a particular therapeutic composition or treatment regimen. Briefly, peritoneal adhesions occur in animals as a result of severe inflicted damage, which usually involves two adjacent surfaces. Injuries may be mechanical, due to ischemia, or due to the introduction of foreign material. Mechanical injuries include crushing of the bowel (Choate et al., Arch. Surg. 88:249-254,1964) and stripping or scrubbing away the outer layers of bowel wall (Gustavsson et al., Acta Chir. Scand. 109:327-333, 1955). Dividing major vessels to loops of the intestine induces ischemia (James et al., J. Path. Bact 90:279-287,1965). Foreign material that may be introduced into the area includes talcum (Green et al., Proc. Soc. Exp. Biol. Med. 133:544-550,1970), gauze sponges (Lehman and Boys, Ann. Surg 111:427-435, 1940), toxic chemicals (Chancy, Arch. Surg. 60:1151-1153,1950), bacteria (Moin et al., Am. J. Med. Sci. 250:675-679, 1965) and feces (Jackson, Surgery 44:507-518,1958).

Presently, typical adhesion prevention models include the rabbit uterine horn model, which involves the abrasion of the rabbit uterus (Linsky et al., J. Reprod. Med. 32(1):17-20, 1987), the rabbit uterine horn; devascularization modification model, which involves abrasion and devascularization of the uterus (Wiseman et al., J. Invest Surg. 7:527-532, 1994); and the rabbit cecal sidewall model which involves the excision of a patch of parietal peritoneum plus the abrasion of the cecum (Wiseman and Johns, Fertil. Steril. Suppl: 25S, 1993).

Utilizing the agents, compositions and methods provided herein a wide variety of adhesions and complications of surgery can be treated or prevented. Adhesion formation or unwanted scar tissue accumulation and/or encapsulation complicates a variety of surgical procedures. As described above, surgical adhesions complicate virtually any open or endoscopic surgical procedure in the abdominal or pelvic cavity. Encapsulation of surgical implants also complicates breast reconstruction surgery, joint replacement surgery, hernia repair surgery, artificial vascular graft surgery, and neurosurgery. In each case, the implant becomes encapsulated by a fibrous connective tissue capsule that compromises or impairs the function of the surgical implant (e.g., breast implant, artificial joint, surgical mesh, vascular graft, dural patch). Chronic inflammation and scarring also occurs during surgery to correct chronic sinusitis or removal of other regions of chronic inflammation (e.g., foreign bodies; infections such as fungal and mycobacterial).

The compositions of this invention can be administered in any manner that achieves a statistically significant result. Preferred methods include peritubular administration (either direct application at the time of surgery or with endoscopic, ultrasound, CT, MRI, or fluoroscopic guidance); “coating” the surgical implant; and placement of a drug-eluting polymeric implant at the surgical site.

In a general method for coating tissues to prevent the formation of adhesions following surgery, the activated polymer is dissolved in a biologically acceptable buffer that has a pH lower that 6.8. The resultant solution is then applied to the desired tissue surface in the presence of a second biologically acceptable buffer that has a pH greater than 7.5. Application of the reaction mixture to the tissue site may be by extrusion, brushing, spraying or by any other convenient means.

In one embodiment, a multifunctional hydroxysuccinimidyl PEG derivative (e.g., tetra functional poly(ethylene glycol) succinimidyl glutarate) can be applied to a tissue surface. For example, in one embodiment, the multifunctional hydroxysuccinimidyl PEG derivative may be in the form of a solution having a basic pH (e.g., a pH of greater than 8). In one embodiment, the multifunctional hydroxysuccinimidyl PEG derivative is not in admixture with any other tissue reactive compound and/or with any component that will react with the derivative.

Following application of the composition to the surgical site, any excess solution may be removed from the surgical site if deemed necessary. At this point in time, the surgical site can be closed using conventional means (sutures, staples, bioadhesive etc.).

The compostion can also be applied in alternative manners. In one embodiment, the activated polymer can be applied to the surgical site in the solid state. As the polymer hydrates, it can then react with the tissue surface to which it was applied. The reaction with the underlying surface may anticipated to be relatively slow. A biologically acceptable buffer, with a pH greater than 7.5 can be applied to the tissue before and/or after the solid acitvated polymer has been applied.

Use of the Activated Synthetic Polymers to Coat Implants

Another use of the activated polymer compositions of the invention is as a coating material for synthetic implants. In a general method for coating a surface of a synthetic implant, the activated synthetic polymer is applied to the surface of the implant. In the preferred application, the surface of the implant has functional groups present that are able to react with the activated functional groups of the applied polymer. The surface functional groups can be inherent in the composition of the material used to prepare the implant. The surface functional groups may be introduced to the implant by first treating the surface of the implant. The surface treatments that can be used include, but are not limited to, coating the surface with a polymer that comprises the appropriate functional groups, oxidizing the surface (e.g., acid/potassium permanganate treatment), grafting polymers that comprise the appropriate functional groups onto the implant surface, plasma treat or corona treat the implant surface, or irradiation of the implant surface (e.g., gamma, UV, e-beam etc.). A combination of these surface treatments may also be used to introduce the appropriate functional groups into the implant surface. Application of the reaction mixture to the implant surface may be by extrusion, brushing, dipping, spraying (as described above), or by any other convenient means. Following application of the reaction mixture to the implant surface, the reaction with the surface functional groups is allowed to continue until sufficient reaction has been achieved. A further step of removing any solvent may then follow.

Although this method can be used to coat the surface of any type of synthetic implant, it is particularly useful for implants where reduced thrombogenicity is an important consideration, such as artificial blood vessels and heart valves, vascular grafts, vascular stents, catheters and stent/graft combinations. The method may also be used to coat implantable surgical membranes (e.g., monofilament polypropylene) or meshes (e.g., for use in hernia repair). Breast implants may also be coated using the above method in order to minimize capsular contracture. The compositions of the present invention may also be used to coat lenticules, which are made from either naturally occurring or synthetic polymers.

Tumor Excision Sites

Within further aspects of the present invention, methods are provided for treating tumor excision sites, comprising administering to a patient an activated polymer composition comprising a anti-microtubule agent, such that the local recurrence of cancer is inhibited.

Local recurrence of malignancy following primary surgical excision of the mass remains a significant clinical problem. In one series of breast cancer patients who underwent lumpectomy of a primary breast tumor, almost ⅔ of the patients that presented with recurrent disease had local (i.e., tumor in the same breast) disease, while only ⅓ presented with metastatic disease. Other pathological studies have demonstrated that most local tumor recurrence occurs within a 2 cm margin of the primary resection margin. Therefore, treatments designed to address this problem are greatly needed. Local recurrence is also a significant problem in the surgical management of brain tumors. For example, within one embodiment of the invention, anti-microtubule compositions may be administered to the site of a neurological tumor subsequent to excision, such that recurrence of the brain tumor (benign or malignant) is inhibited. Briefly, the brain is highly functionally localized; i.e., each specific anatomical region is specialized to carry out a specific function. Therefore it is the location of brain tumor pathology that is often more important than the type. A relatively small lesion in a key area can be far more devastating than a much larger lesion in a less important area. Similarly, a lesion on the surface of the brain may be easy to resect surgically, while the same tumor located deep in the brain may not (one would have to cut through too many vital structures to reach it). Also, even benign tumors can be dangerous for several reasons: they may grow in a key area and cause significant damage; even though they would be cured by surgical resection this may not be possible; and finally, if left unchecked they can cause increased intracranial pressure. The skull is an enclosed space incapable of expansion. Therefore, if something is growing in one location, something else must be being compressed in another location-the result is increased pressure in the skull or increased intracranial pressure. If such a condition is left untreated, vital structures can be compressed, resulting in death. The incidence of CNS (central nervous system) malignancies is 8-16 per 100,000. The prognosis of primary malignancy of the brain is dismal, with a median survival of less than one year, even following surgical resection. These tumors, especially gliomas, are predominantly a local disease that recurs within 2 centimeters of the original focus of disease after surgical removal.

Representative examples of brain tumors which may be treated utilizing the compositions and methods described herein include Glial Tumors (such as Anaplastic Astrocytoma, Glioblastoma Multiform, Pilocytic Astrocytoma, Oligodendroglioma, Ependymoma, Myxopapillary Ependymoma, Subependymoma, Choroid Plexus Papilloma); Neuron Tumors (e.g., Neuroblastoma, Ganglioneuroblastoma, Ganglioneuroma, and Medulloblastoma); Pineal Gland Tumors (e.g., Pineoblastoma and Pineocytoma); Menigeal Tumors (e.g., Meningioma, Meningeal Hemangiopericytoma, Meningeal Sarcoma); Tumors of Nerve Sheath Cells (e.g., Schwannoma (Neurolemmoma) and Neurofibroma); Lymphomas (e.g., Hodgkin's and Non-Hodgkin's Lymphoma (including numerous subtypes, both primary and secondary); Malformative Tumors (e.g., Craniopharyngioma, Epidermoid Cysts, Dermoid Cysts and Colloid Cysts); and Metastatic Tumors (which can be derived from virtually any tumor, the most common being from lung, breast, melanoma, kidney, and gastrointestinal tract tumors).

As noted above, representative drugs (e.g., anti-microtubule agents) for treating adhesions are discussed in detail above, and include taxanes, colchicine and CI 980 (Allen et al., Am. J. Physiol. 261(4 Pt. 1): L315-L321, 1991; Ding et al., J. Exp. Med. 171(3): 715-727,1990; Gonzalez et al., Exp. Cell. Res. 192(1): 10-15, 1991; Stargell et al., Mol. Cell. Biol. 12(4): 1443-1450,1992; Garcia et al., Antican. Drugs 6(4): 533-544,1995), vinca alkaloids (e.g., vinblastine and vincristine), discodermolide (ter Haar et al., Biochemistry 35: 243-250, 1996), as well as analogues and derivatives of any of these

Within one embodiment of the invention, the compound or composition is administered directly to the tumor excision site (e.g., applied by swabbing, brushing, spraying or otherwise coating the resection margins of the tumor with the antimicrotubule composition(s)). Within particularly preferred embodiments of the invention, the antimicotubule compositions are applied after hepatic resections for malignancy, colon tumor resection surgery, breast tumor lumpectomy and after neurosurgical tumor resection operations.

For paclitaxel, a variety of embodiments are described for the management of local tumor recurrence. In one preferred embodiment, 1-25 mg of paclitaxel is loaded into a microsphere carrier, incorporated into activated polymer composition and applied to the resection surface as a solution, powder, “paste”, “film”, or “gel” which releases the drug over a period of time such that the incidence of tumor recurrence is reduced. During endoscopic procedures, 1-25 mg of paclitaxel contained in the microsphere-avtivated polymer preparation is applied as a “spray”, via delivery ports in an endoscope, to the resection site. In another embodiment, an intraperitoneal surgical lavage fluid containing 10 to 250 mg paclitaxel is administered at the time of, or immediately following, surgery.

For docetaxel, a variety of embodiments are described for the management of local tumor recurrence. In one preferred embodiment, 0.5-15 mg of docetaxel is loaded into a microsphere carrier, incorporated into activated polymer composition and applied to the resection surface as a solution, powder, “paste”, “film”, or “gel” which releases the drug over a period of time such that the incidence of tumor recurrence is reduced. During endoscopic procedures, 0.5-15 mg of docetaxel contained in the micellar-hyaluronic acid preparation is applied as a “spray”, via delivery ports in an endoscope, to the resection site. In another embodiment, an intraperitoneal surgical lavage fluid containing 10 to 100 mg docetaxel is administered at the time of, or immediately following, surgery.

Other Uses for the Activated Synthetic Polymers

The activated polymer compositions of the invention can also be coated onto the interior surface of a physiological lumen, such as a blood vessel or Fallopian tube, thereby serving as a sealant to prevent stenosis restenosis of the lumen following medical treatment, such as, for example, balloon catheterization to remove arterial plaque deposits from the interior surface of a blood vessel, or removal of scar tissue or endometrial tissue from the interior of a Fallopian tube. A thin layer of the reaction mixture is preferably applied to the interior surface of the vessel (for example, via catheter). Because the compositions of the invention are not readily degradable in vivo, the potential for restenosis due to degradation of the coating is minimized. The use of crosslinked polymer compositions having a net neutral charge further minimizes the potential for restenosis.

The activated polymer compositions of the invention can also be applied to surfaces to reduce the “fogging” of the surface to which it was applied (e.g., mirrors, ski goggles, glasses etc).

The activated polymer composition of this invention can also be applied to a surface to enhance the lubricity of the surface. This can be useful in, for example, catheter or contact lens applications. In a general method for coating a surface of a medical device, the activated synthetic polymers is applied to the surface of the device. In the preferred application, the surface of the device has functional groups present that are able to react with the activated functional groups of the applied polymer. The surface functional groups can be inherent in the composition of the material used to prepare the implant. The surface functional groups may be introduced to the implant by first treating the surface of the implant. The surface treatments that can be used include, but are not limited to, coating the surface with a polymer that comprises the appropriate functional groups (e.g., chitosan, poly(ethyleneimine), oxidizing the surface (e.g., acid/potassium permanganate treatment), grafting polymers that comprise the appropriate functional groups onto the implant surface, plasma treat or corona treat the implant surface, or irradiation of the implant surface (e.g., gamma, UV, e-beam etc.). A combination of these surface treatments may also be used to introduce the appropriate functional groups into the implant surface. Application of the reaction mixture to the implant surface may be by extrusion, brushing, dipping, spraying (as described above), or by any other convenient means. Following application of the reaction mixture to the implant surface, the reaction with the surface functional groups is allowed to continue until sufficient reaction has been achieved. A further step of removing any solvent may then follow.

### EXAMPLES

The following examples are put forth so as to provide those of ordinary skill in the art with a complete disclosure and description of how to make the preferred embodiments of the conjugates, compositions, and devices and are not intended to limit the scope of what the inventors regard as their invention. Efforts have been made to ensure accuracy with respect to numbers used (e.g., amounts, temperature, molecular weight, etc.) but some experimental errors and deviation should be accounted for. Unless indicated otherwise, parts are parts by weight, molecular weight is weight average molecular weight, temperature is in degrees Centigrade, and pressure is at or near atmospheric.

### Example 1

### Reactive Compounds for Inclusion with Secondary Carriers

In one aspect of the present invention, a biologically active compound (drug) may be incorporated into a secondary carrier, and this drug/carrier combination is combined with a synthetic polymer comprising multiple activated groups. This is particularly useful in those instances where the drug is hydrophobic, and the carrier facilitates water solubility or dispersibility of the drug. Furthermore, this is particularly useful in those instances where the synthetic polymer (with which the drug will be combined) is water soluble and/or dispersible, and will be present as an aqueous composition when it is contacted with the surface (tissue or device surface). In such instances, it may be desirable to have the secondary carrier react with the synthetic polymer comprising multiple activated groups. In order for this reaction to occur, the secondary carrier must have reactive functional groups. The following synthetic schemes provides compounds that may be included within a secondary carrier, e.g., a nanosphere, micelle, or the like, where these compounds have reactive functional groups.

A. R=C₁₇ (Thiol Functional Hydrocarbon)

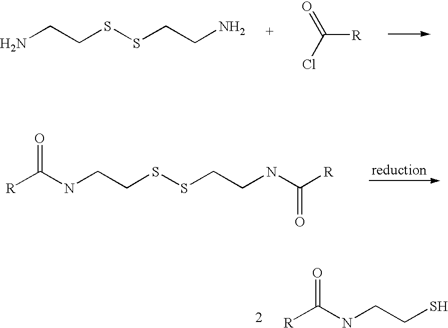

To a cooled solution of cystamine (5 mmol) and triethylamine (15 mmol) in 25 mL methylene chloride in a dry 50 mL round bottom flask equipped with magnetic stirrer, rubber septum and nitrogen balloon was slowly added stearoyl chloride (10 mmol). The mixture was allowed to warm up to room temperature and stirred for 4 hours. After filtration of the trimethylammonium salts, the organic solution was washed with water and dried over Mg₂SO₄. The solvent was evaporated to yield N,N′-bis-stearoyl-cystamine that was purified by silica gel chromatography. The disulfide linkage was reduced using ten fold molar excess of 10 mM triphenylphosphine in methylene chloride under nitrogen atmosphere at room temperature overnight.

B. R=PEG (Thiol Functional PEG)

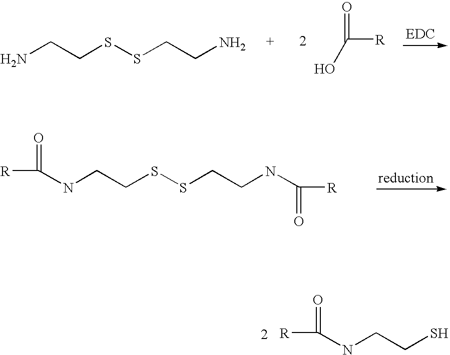

The coupling of 10 mmol PEG-carboxylate and 5 mmol cystamine in the presence of 11 mmol 1-Ethyl-3-(3-dimethylaminopropyl)-carbodiimide (EDC) was carried out at room temperature at pH 4 in 2 hours. The solution was dialyzed against distilled water in a 1 kDa molecular weight cut off membrane overnight and the product was isolated by lyophilization. The disulfide linkage was reduced by 10 fold molar excess of 10 mM dithiothreitol at pH 8.5 under nitrogen.

C. R=C₁₉ (Thiol functional Hydrocarbon)

The coupling of 10 mmol eicosanoic acid and 5 mmol cystamine in the presence of 11 mmol dicyclohexyl-carbodiimide (DCC) was carried out at room temperature in methylene chloride over four hours under anhydrous conditions. The solution was filtered and the solvent was evaporated under vacuum. Purification was carried out by precipitation in methanol. The disulfide linkage was reduced by ten fold molar excess of 10 mM triphenyl phosphine in methylene chloride under nitrogen.

D. R=C₁₁ (Thiol Functional Hydrocarbon)

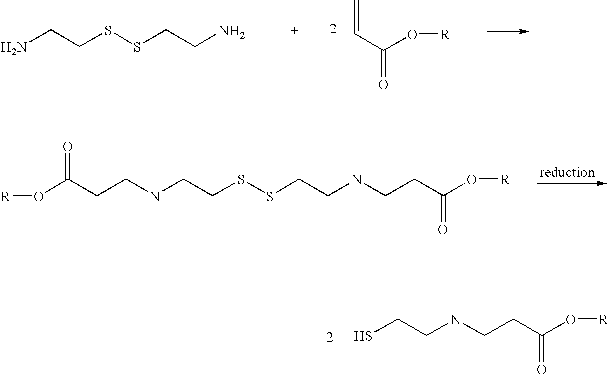

Lauryl acrylate (10 mmol) and methoxyphenol (2 mg) were dissolved in 10 mL chloroform, purged with nitrogen and cooled in an ice-bath. Cystamine (5 mmol) was added and the reaction mixture was stirred overnight covered from light at room temperature. The product was precipitated in methanol. After removal of the solvent the disulfide linkage was reduced using ten fold molar excess of 10 mM triphenylphosphine in methylene chloride under nitrogen atmosphere at room temperature overnight.

E. R=PEG (Thiol Functional PEG)

PEG-acrylate (10 mmol) and methoxyphenol (2 mg) were dissolved in 10 mL distilled water, purged with nitrogen and cooled in an ice-bath. Cystamine (5 mmol) was added and the reaction mixture was stirred overnight covered from light at room temperature. The solution was dialyzed against distilled water in a 1 kDa molecular weight cut off membrane overnight and the product was isolated by lyophilization. The disulfide linkage was reduced by ten fold molar excess of 10 mM dithiothreitol at pH 8.5 under nitrogen.

F. R=C₁₈ (Thiol Functional Hydrocarbon)

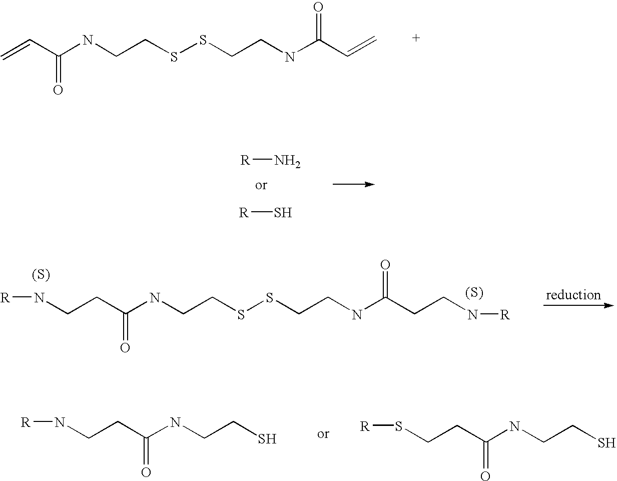

N,N′-bis(acryloyl) cystamine (5 mmol) and methoxyphenol (2 mg) were dissolved in 10 mL chloroform, purged with nitrogen and cooled in an ice-bath. Octadecyl amine or octadecyl mercaptan (10 mmol) was added and the reaction mixture was stirred overnight covered from light at room temperature. The product was precipitated in methanol. After evaporation of the solvent, the disulfide linkage was reduced using ten fold molar excess of 10 mM triphenylphosphine in methylene chloride under nitrogen atmosphere at room temperature overnight.

G. R=PEG (Thiol Functional PEG)

N,N′-bis(acryloyl) cystamine (5 mmol) and methoxyphenol (2 mg) were dissolved in 10 mL distilled water, purged with nitrogen and cooled in an ice-bath. Amino or sulfhydril PEG (10 mmol) was added and the reaction mixture was stirred overnight covered from light at room temperature. The solution was dialyzed against distilled water in a 1 kDa molecular weight cut off membrane overnight and the product was isolated by lyophilization. The disulfide linkage was reduced by ten fold molar excess of 10 mM dithiothreitol at pH 8.5 under nitrogen.

H. R=PEG (Thiol Functional PEG)

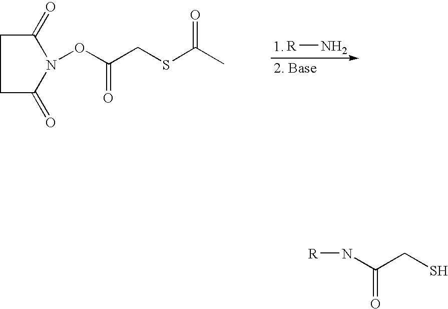

The reaction of amino-PEG with five fold molar excess of succinimidyl acetyl thioacetate (SATA) was carried out in a pH 9 sodium bicarbonate-sodium phosphate buffer at room temperature in 1 hour. SATA was previously dissolved in dimethyl formamide (10 mg/mL) immediately prior to use and slowly added to the PEG solution during vigorous stirring. The functionalized PEG product was separated by gel filtration chromatography on a Sephadex G10 column. After lyophilization the thioester group was removed by 50 mM hydroxylamine at neutral pH.

I. R=C₁₈

The reaction of ocatdecyl amine with two fold molar excess of succinimidyl acetyl thioacetate (SATA) was carried out in dimethyl formamide at room temperature overnight under anhydrous conditions. SATA was previously dissolved in dimethyl formamide (10 mg/mL) immediately prior to use and slowly added to the hydrocarbon solution during vigorous stirring. The functionalized product was separated by precipitating in methanol. The thioester group was removed by 50 mM hydroxylamine at neutral pH.

J. R=PEG (Thiol Functional PEG)

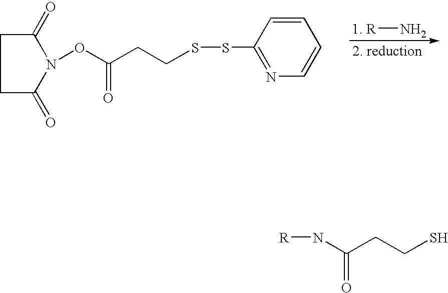

R—SH can be used to produce thiol functional molecules with a thioester linker similarly to above. For example, the reaction of amino-PEG with five fold molar excess of succinimidyl 3-(2-pyridylthio) propionate (SPDP) was carried out in a pH 9 sodium bicarbonate-sodium phosphate buffer at room temperature in 1 hour. SPDP was previously dissolved in dimethyl formamide (10 mg/mL) immediately prior to use and slowly added to the PEG solution during vigorous stirring. The functionalized PEG product was separated by gel filtration chromatography on a Sephadex G10 column. After lyophilization the disulfide bond was reduced with ten fold molar excess of 10 mM dithiothreitol at pH 8.5 under nitrogen.

K. R=C₁₈

The reaction of octadecyl amine with equimolar succinimidyl 3-(2-pyridylthio) propionate (SPDP) was carried out in dimethyl formamide at room temperature overnight under anhydrous conditions. SPDP was previously dissolved in dimethyl formamide (10 mg/mL) immediately prior to use and slowly added to the hydrocarbon solution during vigorous stirring. The functionalized product was separated by precipitation in methanol. The disulfide bond was reduced with ten fold molar excess of 10 mM dithiothreitol under nitrogen in chloroform.

L. R=C₁₁ (Amino Functional PEG-Hydrocarbon Block)

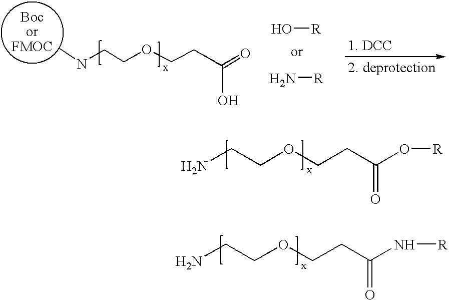

R—SH can be used to produce thiol functional molecules with a thioester linker similarly to above. For example, the coupling of 5 mmol protected amino-PEG-carboxylate and 5 mmol lauryl alcohol in the presence of 11 mmol dicyclohexyl-carbodiimide (DCC) was carried out at room temperature in toluene over four hours. The solution was filtered and the solvent was evaporated under vacuum. Purification could be carried out on a silicagel column. The BOC protecting group could be removed by 50% TFA in dichloromethane.

M. R=PLGA (Amino Functional PEG-PLGA Block)

The coupling of 5 mmol protected amino-PEG-carboxylate and 5 mmol PLGA in the presence of 5.5 mmol 1-Ethyl-3-(3-dimethylaminopropyl)-carbodiimide (EDC) was carried out at room temperature at pH 4 in 2 hours. The solution was dialyzed against distilled water in a 1 kDa molecular weight cut off membrane overnight and the product was isolated by lyophilization. The FMOC protecting group was removed by a 20% piperidine in DMF.

N. R=Polymer (Amino Functional PEG-Polymer Block)

Compounds of this structure may be prepared in a manner analogous to that described in Example 1M above.

O. R=Lipid (Amino Functional PEG-Lipid Block)

Compounds of this structure may be prepared in a manner analogous to that described in Example 1M above.

P. Q=CH₃, Initiating Group OH.

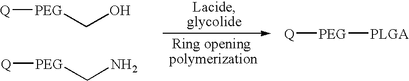

For the ring opening polymerization of D,L-lactide, 40 g lactide was weighed into a 250 mL round bottom flask with 60 g methoxy poly(ethylene glycol) (MePEG, MW=2000). The reagents were vacuum dried overnight; the flask was flushed with nitrogen and placed into a 130° C. oil bath while stirring. After the reagents melted, 300 mg stannous 2-ethyl hexanoate was added to initiate polymerization. After 5 hours the polymer was poured into a metal tray to solidify.

Q. Q=COOH, Initiating Group NH₂.

For the ring opening polymerization of D,L-lactide, 25 g lactide dried overnight under vacuum in a 250 mL round bottom flask was mixed with 250 mg 12-aminoundecanoic acid. The flask was flushed with nitrogen and placed into a 130° C. oil bath while stirring. After the reagents melted, 100 mg stannous 2-ethyl hexanoate was added to initiate polymerization. After 2 hours the viscous polymer was poured into a metal tray to solidify. In a similar manner, Q can be protected amine or thiol to produce functional blocks.

R. R=Any of Above

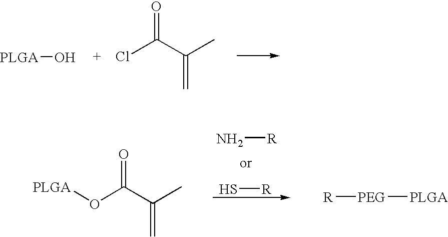

Glassware was flame dried and anhydrous conditions were used during the esterification reaction. Dry PLGA (1 equivalent OH) was weighed into the reaction flask containing anhydrous methylene chloride (0.6 ml/mmol) and 1 molar equivalent triethyl amine. The mixture was purged with nitrogen while cooling in an ice bath. The acid chloride (1.3 equivalent) was added via syringe in increments. After the addition the mixture was stirred for two hours and poured into three-fold volume of distilled water. The aqueous layer was washed with methylene chloride and the combined organic layer was washed with NaHCO₃. After drying with Mg₂SO₄ and filtration 2 mg hydroquinone was added and the solvent was removed by vacuum. Similarly, the resulting methacryloyl function can undergo the reactions described with acrylates.

### Example 2

### Effect of Buffer PH on Adhesion Reduction

Sample Preparation and Administration

Tetra functional poly (ethylene glycol) succinimidyl glutarate (4-arm-NHS-PEG, Cat # P4SG-10, Sunbio Inc., Anyang City, Korea) was weighed into 1 mL plastic syringes (100 mg each), sealed into foil bags with a desiccant and sterilized by gamma-irradiation. Buffers at 8, 8.5 and 9 were prepared by combining various amounts of 0.3 M sodium carbonate and 0.3 M monobasic sodium phosphate. These buffers were freshly prepared before an experiment and sterilized by filtration through a 0.22 micron syringe filter. Sprague-Dawley rats (400-500 g each, n=4) were used in the rat cecal-wall abrasion surgical adhesion model for each pH value (see General Procedure A).

At the time of application, under sterile conditions using sterile equipment, the 4-arm-NHS-PEG was completely dissolved in 0.5 mL sterile water through syringes coupled with a fluid dispensing connector (BBraun Medical Inc., Kirkland, PQ). The syringe containing the 4-arm-NHS-PEG solution and another syringe containing 0.5 mL of buffer, having the appropriate pH, were attached to a Fibrijet surgical sealant applicator with a sealant applicator spray tip (Micromedics Inc., Eagan, Minn.) and this formulation was sprayed onto the injured area. The spraying was done in such a manner as to cover the sidewall and the cecum completely with a layer of the composition. After one minute the animal was surgically closed and allowed to recover.

Results

The percent adhesion and adhesion tenacity scores are summarized in Table 1.

TABLE 1

| Sample Group | Percent Adhesion | Adhesion Tenacity |
|---|---|---|
| Control | 100 ± 0 | 2.18 ± 0.07 |
| pH 8 - 4-arm succinimidyl PEG | 58.7 ± 28.69 | 1.09 ± 0.52 |
| pH 8.5 - 4-arm succinimidyl | 70.75 ± 14.22 | 1.24 ± 0.46 |
| PEG | | |
| pH 9 - 4-arm succinimidyl PEG | 56.75 ± 40.85 | 1.21 ± 0.91 |

These results demonstrate that this composition has the ability to reduce the percent adhesions as well as the severity of the adhesions at any of three different pHs (8, 8.5 and 9).

### Example 3

### Effect of Polymer Concentration on Adhesion Reduction

Sample Preparation and Administration

Tetra functional poly(ethylene glycol) succinimidyl glutarate (4-arm-NHS-PEG, Cat # P4SG-10, Sunbio Inc., Anyang City, Korea) was weighed into 1 mL plastic syringes (either 200 mg, 300 mg or 400 mg was placed into each syrings) in a silica gel dried atmosbag (Aldrich, Milwaukee, Wis.), sealed into foil bags with desiccant and sterilized by gamma-irradiation. The buffer (0.3M sodium carbonate in 0.3M monobasic sodium phosphate mixed to pH 9.2) was freshly prepared and sterilized by filtration through a 0.22 micron syringe filter. Sprague-Dawley rats (400-500 g each, n=4) were used in the rat cecal-wall abrasion surgical adhesion model described in General Procedure A for each polymer concentration value.

At the time of application under sterile conditions using sterile equipment, the 4-arm-NHS-PEG was completely dissolved in 0.5 mL sterile water through syringes coupled with a fluid dispensing connector (BBraun Medical Inc., Kirkland, PQ). The syringe containing the 4-arm-NHS-PEG solution and another syringe containing 0.5 mL of buffer were attached to an air-assisted spray applicator (Micromedics Inc., Eagan, Minn.) and this formulation was sprayed onto the injury area. The spraying was done in such a manner as to cover the side wall and the cecum completely with a layer of the composition. After one minute the animal was surgically closed and allowed to recover.

Results

The percent adhesion and adhesion tenacity scores are summarized in Table 2.

TABLE 2

| Sample Group | Percent Adhesion | Adhesion Tenacity |
|---|---|---|
| Control | 100 ± 0 | 2.12 ± 0.06 |
| 200 mg 4-arm succinimidyl PEG | 61.25 ± 27.2 | 1.19 ± 0.06 |
| 300 mg 4-arm succinimidyl PEG | 43.5 ± 43.2 | 0.78 ± 0.9 |
| 400 mg 4-arm succinimidyl PEG | 52.5 ± 41.1 | 0.925 ± 0.8 |

These results demonstrate that this composition has the ability to reduce the percent adhesions as well as the severity of the adhesions at any of three different polymer concentrations (200 mg, 300 mg or 400 mg in 1.0 mL solution (1:1 water:buffer).

### Example 4

### Preparation of Microspheres with and without Paclitaxel

A) PVA Solution Preparation

1. In a 1000 ml beaker, 1000 ml of distilled water and 100 g of PVA (Aldrich 13-23K, 98% hydrolyzed) are weighed. A two-inch stirrer bar is placed into the beaker. The suspension is heated up to 75-80° C. during stirring. The PVA is dissolved completely (should form a clear solution).

2. The 10% PVA solution (w/v) is cooled down to room temperature and filtered through a syringe in-line filter. Stored at 2-8° C. for use.

B) PLGA solution preparation with or without paclitaxel

1. Appropriate amount of paclitaxel and PLGA (for a total of 1.0 g) are weighed and transferred into the 20 ml scintillation vial.

2. 10 mL of HPLC grade dichloromethane (DCM) is added into the vial to dissolve the PLGA with or without paclitaxel.

3. The polymer with or without paclitaxel is dissolved in DCM by placing the vial on an orbital shaker. The orbital shaker is set at 4.

Preparation of the Microspheres with Diameter Less Than 25 mm

1. 100 ml of 10% PVA solution is transferred into a 400 ml beaker. The beaker is secured by a double side adhesive tape onto the fume-hood. A peddler with 3 blades is placed into the beaker with 0.5 cm above the bottom. The motor is turned on to 2.5 (Dyna-Mix from Fisher Scientific) at first. The 10 ml PLGA/paclitaxel solution is poured into the PVA solution during agitation. Gradually turn up the agitation rate to 5.0. The stirring is maintained for 2.5 to 3.0 hours.

2. The obtained microspheres are filtered through a set of sieves with 53 mm (top) and 25 mm (bottom) into a 100 ml beaker. The microspheres are washed using distilled water while filtering. The filtered microspheres are centrifuged (1000 rpm, 10 min.) and re-suspended/washed with 100 ml distilled water three times to clean the PVA.

3. The washed microspheres are transferred into the freeze-dried beaker using a small amount of distilled water (20-30 ml). The beaker is then sealed and placed into a −20° C. freezer over night.

4. The frozen microspheres are then freeze-dried using a freeze-drier for about 3 days. The dried microspheres are transferred into 20 ml scintillation vial and stored at −20° C.

In a similar manner described above, other biologically active agents, as described above, can be incorporated into a microsphere formulation.

### Example 5

### Incorporation of Funtionalized Groups into Microspheres

Microsphere formulations can be prepared as described above using a PLGA polymer and one of the reagents synthesized in Example 1 above.

### Example 6

### Device Surface Coating—Chitosan Base-Coat

A 1% (w/v) chitosan solution is prepared using 0.2% (v/v) acetic acid. A piece of catheter tubing is dipped into the chitosan solution and is allowed to incubate for 10 minutes. The catheter tubing is removed and then air dried. The chitosan-coated catheter is then immersed into a freshly prepared 10% solution (pH about 8) of tetra functional poly(ethylene glycol) succinimidyl glutarate (4-arm-NHS-PEG, Cat # P4SG-10, Sunbio Inc., Anyang City, Korea) for 5 minutes. The tubing is removed and air-dried. The coated tubing is then rinsed with deionized water and is allowed to air dry. The sample is then further dired under vacuum.

### Example 7

### Device Surface Coating—PEI Base-Coat

A 5% (w/v) polyethyleneimine (PEI] solution is prepared using using deionized water. A piece of catheter tubing is dipped into the PEI solution and is allowed to incubate for 10 minutes. The catheter tubing is removed and then air dried. The PEI-coated catheter is then immersed into a freshly prepared 10% solution (pH about 8) of tetra functional poly(ethylene glycol) succinimidyl glutarate (4-arm-NHS-PEG, Cat# P4SG-10, Sunbio Inc., Anyang City, Korea) for 5 minutes. The tubing is removed and air-dried. The coated tubing is then rinsed with deionized water and is allowed to air dry. The sample is then further dried under vacuum.

### Example 8

### Screening Assay for Assessing the Effect of Mitoxantrone on Cell Proliferation

Fibroblasts at 70-90% confluency are trypsinized, replated at 600 cells/well in media in 96-well plates and allowed to attachment overnight. Mitoxantrone is prepared in DMSO at a concentration of 102 M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and mitoxantrone are incubated at 37° C. for 72 hours (In vitro toxicol. (1990) 3: 219; Biotech. Histochem. (1993) 68: 29; Anal. Biochem. (1993)213: 426).

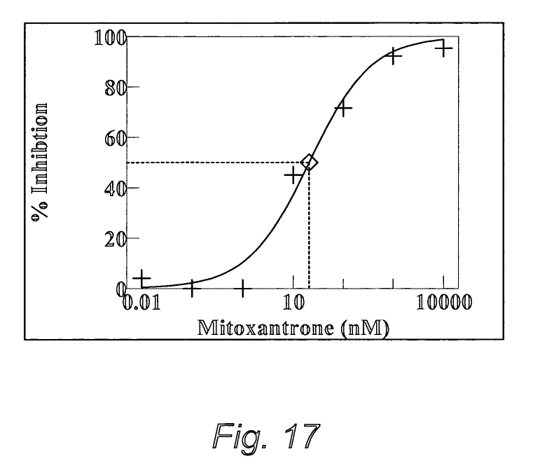

To terminate the assay, the media is removed by gentle aspiration. A 1/400 dilution of CYQUANT 400× GR dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Inhibitory concentration of 50% (IC₅₀) is determined by taking the average of triplicate wells and comparing average relative fluorescence units to the DMSO control. An average of n=4 replicate experiments is used to determine IC₅₀ values. The results of the assay are shown in FIG. 17. (IC₅₀=20 nM for proliferation of human fibroblasts).

### Example 9

### Screening Assay for Assessing the Effect of Mitoxantrone on Nitric Oxide Production by Macrophages

The murine macrophage cell line RAW 264.7 is trypsinized to remove cells from flasks and plated in individual wells of a 6-well plate. Approximately 2×10⁶ cells are plated in 2 mL of media containing 5% heat-inactivated fetal bovine serum (FBS). RAW 264.7 cells are incubated at 37° C. for 1.5 hours to allow adherence to plastic. Mitoxantrone is prepared in DMSO at a concentration of 10⁻² M and serially diluted 10-fold to give a range of stock concentrations (10⁻⁸ M to 10⁻² M). Media is then removed and cells are incubated in 1 ng/mL of recombinant murine IFNγ and 5 ng/mL of LPS with or without mitoxantrone in fresh media containing 5% FBS. Mitoxantrone is added to cells by directly adding mitoxantrone DMSO stock solutions, prepared earlier, at a 1/1000 dilution, to each well. Plates containing IFNγ, LPS plus or minus mitoxantrone are incubated at 37° C. for 24 hours (Chem. Ber. (1879) 12: 426; J. AOAC (1977) 60-594; Ann. Rev. Biochem. (1994) 63: 175).

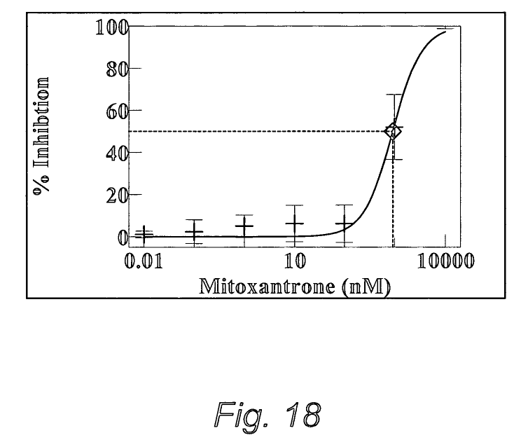

At the end of the 24 hour period, supernatants are collected from the cells and assayed for the production of nitrites. Each sample is tested in triplicate by aliquoting 50 μof supernatant in a 96-well plate and adding 50 μL of Greiss Reagent A (0.5 g sulfanilamide, 1.5 mL H₃PO₄, 48.5 mL ddH₂O) and 50 μL of Greiss Reagent B (0.05 g N-(1-Naphthyl)-ethylenediamine, 1.5 mL H₃PO₄, 48.5 mL ddH₂O). Optical density is read immediately on microplate spectrophotometer at 562 nm absorbance. Absorbance over triplicate wells is averaged after subtracting background and concentration values are obtained from the nitrite standard curve (1 μM to 2 mM). Inhibitory concentration of 50% (IC₅₀) is determined by comparing average nitrite concentration to the positive control (cell stimulated with IFNγ and LPS). An average of n=4 replicate experiments is used to determine IC₅₀ values for mitoxantrone. The results of the assay are shown in FIG. 18. (Mitoxantrone IC₅₀=927 nM for Greiss assay in RAW 264.7 cells.)

### Example 10

### Screening Assay for Assessing the Effect of Bay11-7082 on TNF-Alpha Production by Macrophages

The human macrophage cell line, THP-1 is plated in a 12 well plate such that each well contains 1×10⁶ cells in 2 mL of media containing 10% FCS. Opsonized zymosan is prepared by resuspending 20 mg of zymosan A in 2 mL of ddH₂O and homogenizing until a uniform suspension is obtained. Homogenized zymosan is pelleted at 250 g and resuspended in 4 mL of human serum for a final concentration of 5 mg/mL. and incubated in a 37° C. water bath for 20 minutes to enable opsonization. Bay 11-7082 is prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol Meth. (2000) 235 (1-2): 33-40).

THP-1 cells are stimulated to produce TNFα by the addition of 1 mg/mL opsonized zymosan. Bay 11-7082 is added to THP-1 cells by directly adding DMSO stock solutions, prepared earlier, at a 1/1000 dilution, to each well. Each drug concentration is tested in triplicate wells. Plates are incubated at 37° C. for 24 hours.

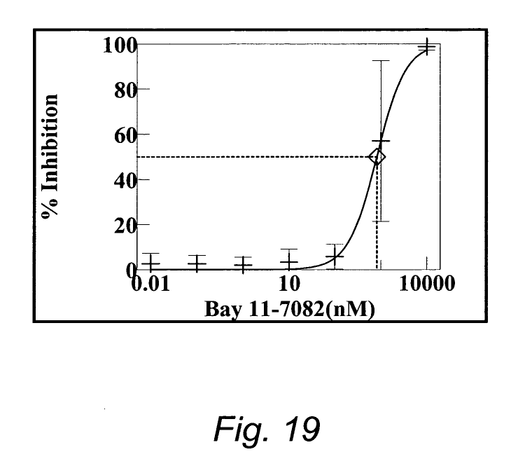

After a 24 hour stimulation, supernatants are collected to quantify TNFα production. TNFα concentrations in the supernatants are determined by ELISA using recombinant human TNFα to obtain a standard curve. A 96-well MaxiSorb plate is coated with 100 μL of anti-human TNFα Capture Antibody diluted in Coating Buffer (0.1M Sodium carbonate pH 9.5) overnight at 4° C. The dilution of Capture Antibody used is lot-specific and is determined empirically. Capture antibody is then aspirated and the plate washed 3 times with Wash Buffer (PBS, 0.05% Tween-20). Plates are blocked for 1 hour at room temperature with 200 μL/well of Assay Diluent (PBS, 10% FCS pH 7.0). After blocking, plates are washed 3 times with Wash Buffer. Standards and sample dilutions are prepared as follows: (a) sample supernatants are diluted 1/8 and 1/16; (b) recombinant human TNFα is prepared at 500 pg/mL and serially diluted to yield as standard curve of 7.8 pg/mL to 500 pg/mL. Sample supernatants and standards are assayed in triplicate and are incubated at room temperature for 2 hours after addition to the plate coated with Capture Antibody. The plates are washed 5 times and incubated with 100 μL of Working Detector (biotinylated anti-human TNFα detection antibody+avidin-HRP) for 1 hour at room temperature. Following this incubation, the plates are washed 7 times and 100 μL of Substrate Solution (Tetramethylbenzidine, H₂O₂) is added to plates and incubated for 30 minutes at room temperature. Stop Solution (2 N H₂SO₄) is then added to the wells and a yellow colour reaction is read at 450 nm with A correction at 570 nm. Mean absorbance is determined from triplicate data readings and the mean background is subtracted. TNFα concentration values are obtained from the standard curve. Inhibitory concentration of 50% (IC₅₀) is determined by comparing average TNFα concentration to the positive control (THP-1 cells stimulated with opsonized zymosan). An average of n=4 replicate experiments is used to determine IC₅₀ values for Bay 11-7082. See FIG. 19. (Bay 11-7082 IC₅₀=810 nM TNFα Production by THP-1 cells).

### Example 11

### Rabbit Surgical Adhesions Model to Assess Fibrosis Inhibiting Agents

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 12

### Rat Surgical Adhesions Model to Assess Fibrosis Inhibiting Agents

Sprague Dawley rats are prepared for surgery by anaesthetic induction with 5% halothane in an enclosed chamber. Anaesthesia is maintained by nose cone on halothane throughout the procedure and Buprenorphen 0.035 mg/kg is injected intramuscularly. The abdomen is shaved, sterilized, draped and entered via a midline incision. The caecum is lifted from the abdomen and placed on sterile gauze dampened with saline. Dorsal and ventral aspects of the caecum are scraped a total of 45 times over the terminal 1.5 cm using a #10 scalpel blade, held at a 45° angle. Blade angle and pressure are controlled to produce punctuated bleeding, while avoiding severe tissue damage or tearing.

The left side of the abdominal cavity is retracted and everted to expose a section of the peritoneal wall nearest the natural resting caecal location. The exposed superficial layer of muscle (transverses abdominis) is excised over an area of 1.0×1.5 cm². Excision includes portions of the underlying internal oblique muscle, leaving behind some intact and some torn fibres from the second layer. Minor local bleeding is tamponaded until controlled.

A test formulation is deployed at the wounded areas, on the abraded sidewall, between the caecum and sidewall. The formulation is deployed using either a syringe spray system or an air-assisted syringe system. The abraded caecum is then positioned over the sidewall wound and sutured at four points immediately beyond the dorsal corners of the wound edge. The large intestine is replaced in a natural orientation continuous with the caecum. The abdominal incision is closed in two layers with 4-0 silk sutures.

Rats are followed for one week, and then euthanized by lethal injection for post mortem examination to score. Severity of post-surgical adhesions is scored by independently assessing the tenacity and extent of adhesions at the site of caecal-sidewall abrasion, at the edges of the abraded site, and by evaluating the extent of intestinal attachments to the exposed caecum. Adhesions are scored on a scale of 0-4 with increasing severity and tenacity. The extent of adhesion is scored as a percent of the injured area that contained adhesions.

### Example 13

### Inhibition of Surgical Adhesion in a Rabbit Uterine Horn Model

Female New Zealand White rabbits were anesthetized with halothane and prepared for sterile abdominal surgery. A laparotomy was performed and both uterine horns were exteriorized. Each horn was scraped 40 times with a scalpel blade and rubbed with gauze for 2.5 minutes. In six animals the 4-arm-PEG formulation was sprayed evenly over the injured horns. Six other animals were left untreated. The horns were replaced in the abdominal cavity and the abdominal wound was closed in layers. The animals were recovered and kept for 14 days. At that time, the animals were sacrificed with an IV injection of Euthanyl. The abdominal cavity was open and the uterine horns were exposed. Length of adhesion along the uterine horns was recorded. Mean adhesion length was 85+/−19 cm in the control group. Adhesion length was significantly decreased to 34+/−46 cm in the treatment group (p<0.05).

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A composition comprising a synthetic polymer and a drug, the polymer comprising multiple activated groups.

2. The composition of claim 1 wherein the synthetic polymer has a cyclic core.

3. The composition of claim 2 wherein the cyclic core comprises a six-membered carbocyclic group.

4. The composition of claim 2 wherein the cyclic core comprises an inositol residue.

5. The composition of claim 2 wherein the cyclic core comprises a lactitol residue.

6. The composition of claim 2 wherein the cyclic core comprises a sorbitol residue.

7. The composition of claim 1 wherein the synthetic polymer has a branched chain core.

8. The composition of claim 7 wherein the branched chain core is a polyhydric compound residue.

9. The composition of claim 8 wherein the branched chain core is a glycerol residue.

10. The composition of claim 8 wherein the branched chain core is a pentaerythritol residue.

11. The composition of claim 8 wherein the branched chain core is a diglycerol residue.

12. The composition of claim 7 wherein the branched chain core is a poly(carboxylic acid) compound residue.

13. The composition of claim 7 wherein the branched chain core is a polyamine compound residue.

14. The composition of claim 7 wherein the branched chain core comprises polyamino acid.

15. The composition of claim 1 wherein the synthetic polymer comprises poly(alkylene)oxide.

16. The composition of claim 15 wherein the poly(alkylene)oxide comprises ethylene oxide residues.

17. The composition of claim 15 wherein poly(alkylene)oxide comprises propylene oxide residues.

18. The composition of claim 15 wherein the poly(alkylene) oxide has a molecular weight of about 100 to about 100,000.

19. The composition of claim 15 wherein the poly(alkylene) oxide has a molecular weight of about 1,000 to about 20,000.

20. The composition of claim 15 wherein the poly(alkylene) oxide has a molecular weight of about 1,000 to about 15,000.

21. The composition of claim 15 wherein the poly(alkylene) oxide has a molecular weight of about 1,000 to about 10,000.

22. The composition of claim 15 wherein the poly(alkylene) oxide has a molecular weight of about 1,000 to about 5,000.

23. The composition of claim 15 wherein the poly(alkylene) oxide has a molecular weight of about 7,500 to about 20,000.

24. The composition of claim 15 wherein the poly(alkylene) oxide has a molecular weight of about 7,500 to about 15,000.

25. The composition of claim 17 wherein the poly(alkylene) oxide has a molecular weight of about 7,500 to about 20,000.

26. The composition of claim 1 wherein the polymer has 2-12 activated groups.

27. The composition of claim 26 wherein the polymer has 2 activated groups.

28. The composition of claim 26 wherein the polymer has 3 activated groups.

29. The composition of claim 26 wherein the polymer has 4 activated groups.

30. The composition of claim 26 wherein the polymer has 6 activated groups.

31. The composition of claim 26 wherein the polymer has 9 activated groups.

32. The composition of claim 26 wherein the polymer has 12 activated groups.

33. The composition of claim 1 wherein the activated groups are protein-reactive.

34. The composition of claim 33 wherein the activated groups are reactive with hydroxyl groups.

35. The composition of claim 33 wherein the activated groups are reactive with thiol groups.

36. The composition of claim 33 wherein the activated groups are reactive with amino groups.

37. The composition of claim 1 wherein the activated group comprises an electrophilic site.

38. The composition of claim 37 wherein the electrophilic site is a carbonyl group.

39. The composition of claim 1 wherein the activated group comprises a leaving group.

40. The composition of claim 39 wherein the leaving group is an N-oxysuccinimide group.

41. The composition of claim 39 wherein the leaving group is an N-oxymaleimide group.

42. The composition of claim 1 wherein the activated group comprises an electrophilic site adjacent to a leaving group.

43. The composition of claim 42 wherein the electrophilic site is a carbonyl group.

44. The composition of claim 42 wherein the leaving group is selected from N-oxysuccinimide and N-oxymaleimide.

45. The composition of claim 42 wherein the electrophilic group is carbonyl and the leaving group is selected from N-oxysuccinimide and N-oxymaleimide.

46. The composition of claim 1 wherein the synthetic polymer comprises the formula (polymer backbone)-(Q-Y)ₙ wherein Q is a linking group, Y is an activated functional group, and n is an integer of greater than 1.

47. The composition of claim 46 wherein the polymer backbone comprises poly(alkylene) oxide.

48. The composition of claim 46 wherein Q is selected from the group consisting of-G-(CH₂)ₙ— wherein G is selected from O, S, NH, —O—CO— and —O—CO—NH—(CH₂)ₙ; O₂C—CR¹H— wherein R¹ is selected from hydrogen and alkyl; and O—R²—CO—NH wherein R² is selected from CH₂ and CO—NH—CH₂CH₂.

49. The composition of claim 46 wherein n is 2-12.

50. The composition of claim 46 wherein Y comprises an electrophilic cite adjacent to a leaving group.

51. The composition of claim 50 wherein the electrophilic site is a carbonyl group.

52. The composition of claim 50 wherein the leaving group comprises (N—CO—CH₂)₂.

53. The composition of claim 46 wherein the synthetic polymer has the formula (polymer backbone)-(Q-Y)ₙ.

54. The composition of claim 46 wherein a chain extender is located between either (polymer backbone) and Q or between Q and Y.

55. The composition of claim 1 wherein the synthetic polymer comprises the formula (polymer backbone)-(D-Q-Y)ₙ wherein D is a biodegradable group, Q is a linking group, Y is an activated functional group, and n is an integer of greater than 1.

56. The composition of claim 55 wherein the polymer backbone comprises poly(alkylene) oxide.

57. The composition of claim 55 wherein D comprises a chemical group selected from lactide, glycolide, epsilon-caprolactone and poly(alpha-hydroxy acid).

58. The composition of claim 55 wherein D comprises a chemical group selected from poly(amino acid), poly(anhydride), poly(orthoester).

59. The composition of claim 55 wherein Q is selected from the group consisting of -G-(CH₂)ₙ— wherein G is selected from O, S, NH, —O—CO— and —O—CO—NH—(CH₂)ₙ; O₂C—CR¹H— wherein R¹ is selected from hydrogen and alkyl; and O—R²—CO—NH wherein R² is selected from CH₂ and CO—NH—CH₂CH₂.

60. The composition of claim 55 wherein Y comprises an electrophilic cite adjacent to a leaving group.

61. The composition of claim 60 wherein the electrophilic site is a carbonyl group.

62. The composition of claim 60 wherein the leaving group comprises (N—CO—CH₂)₂.

63. The composition of claim 60 wherein the synthetic polymer has the formula (polymer backbone)-(D-Q-Y)ₙ.

64. The composition of claim 55 wherein a chain extender is located between either (polymer backbone) and Q or between Q and Y.

65. The composition of claim 1 comprising first and second polymers comprising multiple activated groups, where the first and second polymers are non-identical.

66. The composition of claim 65 wherein the first and second polymer comprise different activated groups.

67. The composition of claim 65 wherein the first and second polymers have different number average molecular weights.

68. The composition of claim 65 wherein the first and second polymers have a different number of activated groups.

69. The composition of claim 1 wherein the polymer is soluble in water at a concentration of at least 1 grams polymer/99 grams water at 25° C.

70. The composition of claim 69 wherein the polymer is soluble in water at a concentration of at least 2 grams polymer/99 grams water at 25° C.

71. The composition of claim 69 wherein the polymer is soluble in water at a concentration of at least 3 grams polymer/99 grams water at 25° C.

72. The composition of claim 69 wherein the polymer is soluble in water at a concentration of at least 4 grams polymer/99 grams water at 25° C.

73. The composition of claim 69 wherein the polymer is soluble in water at a concentration of at least 5 grams polymer/99 grams water at 25° C.

74. The composition of claim 1 wherein the drug is efficacious in inhibiting one or a combination of cellular activities selected from the group consisting of cell division, cell secretion, cell migration, cell adhesion, inflammatory activator production and/or release, angiogenesis and free radical formation and/or release.

75. The composition of claim 1 wherein the drug is an angiogenesis inhibitor.

76. The composition of claim 1 wherein the drug is a 5-Lipoxygenase inhibitor or antagonist.

77. The composition of claim 1 wherein the drug is a chemokine receptor antagonist.

78. The composition of claim 1 wherein the drug is a cell cycle inhibitor or an analogue or derivative thereof.

79. The composition of claim 78 wherein the cell cycle inhibitor is a microtubule stabilizing agent.

80. The composition of claim 79 wherein the microtubule stabilizing agent is paclitaxel, docetaxel, or Peloruside A.

81. The composition of claim 78 wherein the cell cycle inhibitor is a taxane.

82. The composition of claim 81 wherein the taxane is paclitaxel or an analogue or derivative thereof.

83. The composition of claim 78 wherein the cell cycle inhibitor is an antimetabolite, an alkylating agent, or a vinca alkaloid.

84. The composition of claim 83 wherein the vinca alkaloid is vinblastine, vincristine, vincristine sulfate, vindesine, vinorelbine, or an analogue or derivative thereof.

85. The composition of claim 78 wherein the cell cycle inhibitor is camptothecin or an analogue or derivative thereof.

86. The composition of claim 78 wherein the cell cycle inhibitor is selected from the group consisting of mitoxantrone, etoposide, 5-fluorouracil, doxorubicin, methotrexate, Mitomycin-C, CDK-2 inhibitors, and analogues and derivatives thereof.

87. The composition of claim 1 wherein the drug is a cyclin dependent protein kinase inhibitor or an analogue or derivative thereof.

88. The composition of claim 1 wherein the drug is an EGF (epidermal growth factor) kinase inhibitor or an analogue or derivative thereof.

89. The composition of claim 1 wherein the drug is an elastase inhibitor or an analogue or derivative thereof.

90. The composition of claim 1 wherein the drug is a factor Xa inhibitor or an analogue or derivative thereof.

91. The composition of claim 1 wherein the drug is a farnesyltransferase inhibitor or an analogue or derivative thereof.

92. The composition of claim 1 wherein the drug is a fibrinogen antagonist or an analogue or derivative thereof.

93. The composition of claim 1 wherein the drug is a guanylate cyclase stimulant or an analogue or derivative thereof.

94. The composition of claim 1 wherein the drug is a heat shock protein 90 antagonist or an analogue or derivative thereof.

95. The composition of claim 1 wherein the drug is an HMGCoA reductase inhibitor or an analogue or derivative thereof.

96. The composition of claim 1 wherein the drug is a hydroorotate dehydrogenase inhibitor or an analogue or derivative thereof.

97. The composition of claim 1 wherein the drug is an IKK2 inhibitor or an analogue or derivative thereof.

98. The composition of claim 1 wherein the drug is an IL-1, ICE, or IRAK antagonist or an analogue or derivative thereof.

99. The composition of claim 1 wherein the drug is an IL-4 agonist or an analogue or derivative thereof.

100. The composition of claim 1 wherein the drug is an immunomodulatory agent.

101. The composition of claim 1 wherein the drug is an inosine monophosphate dehydrogenase inhibitor or an analogue or derivative thereof.

102. The composition of claim 1 wherein the drug is a leukotreine inhibitor or an analogue or derivative thereof.

103. The composition of claim 1 wherein the drug is a MCP-1 antagonist or an analogue or derivative thereof.

104. The composition of claim 1 wherein the drug is a MMP inhibitor or an analogue or derivative thereof.

105. The composition of claim 1 wherein the drug is a NF kappa B inhibitor or an analogue or derivative thereof.

106. The composition of claim 1 wherein the drug is a NO antagonist or an analogue or derivative thereof.

107. The composition of claim 1 wherein the drug is a P38 MAP kinase inhibitor or an analogue or derivative thereof.

108. The composition of claim 1 wherein the drug is a phosphodiesterase inhibitor or an analogue or derivative thereof.

109. The composition of claim 1 wherein the drug is a TGF beta Inhibitor or an analogue or derivative thereof.

110. The composition of claim 1 wherein the drug is a thromboxane A2 antagonist or an analogue or derivative thereof.

111. The composition of claim 1 wherein the drug is a TNFa Antagonist, a TACE, or an analogue or derivative thereof.

112. The composition of claim 1 wherein the drug is a tyrosine kinase inhibitor or an analogue or derivative thereof.

113. The composition of claim 1 wherein the drug is a vitronectin inhibitor or an analogue or derivative thereof.

114. The composition of claim 1 wherein the drug is a fibroblast growth factor inhibitor or an analogue or derivative thereof.

115. The composition of claim 1 wherein the drug is a protein kinase inhibitor or an analogue or derivative thereof.

116. The composition of claim 1 wherein the drug is a PDGF receptor kinase inhibitor or an analogue or derivative thereof.

117. The composition of claim 1 wherein the drug is an endothelial growth factor receptor kinase inhibitor or an analogue or derivative thereof.

118. The composition of claim 1 wherein the drug is a retinoic acid receptor antagonist or an analogue or derivative thereof.

119. The composition of claim 1 wherein the drug is a platelet derived growth factor receptor kinase inhibitor or an analogue or derivative thereof.

120. The composition of claim 1 wherein the drug is a fibrinogin antagonist or an analogue or derivative thereof.

121. The composition of claim 1 wherein the drug is an antimycotic agent or an analogue or derivative thereof.

122. The composition of claim 1 wherein the drug is a bisphosphonate or an analogue or derivative thereof.

123. The composition of claim 1 wherein the drug is a phospholipase A1 inhibitor or an analogue or derivative thereof.

124. The composition of claim 1 wherein the drug is a histamine H1/H2/H3 receptor antagonist or an analogue or derivative thereof.

125. The composition of claim 1 wherein the drug is a macrolide antibiotic or an analogue or derivative thereof.

126. The composition of claim 1 wherein the drug is an GPIIb IIIa receptor antagonist or an analogue or derivative thereof.

127. The composition of claim 1 wherein the drug is an endothelin receptor antagonist or an analogue or derivative thereof.

128. The composition of claim 1 wherein the drug is a peroxisome proliferators-activated receptor agonist or an analogue or derivative thereof.

129. The composition of claim 1 wherein the drug is an estrogen receptor agent or an analogue or derivative thereof.

130. The composition of claim 1 wherein the drug is somatostatin or an analogue or derivative thereof.

131. The composition of claim 1 wherein the drug is a JNK Kinase inhibitor or an analogue or derivative thereof.

132. The composition of claim 1 wherein the drug is a melanocortin analogue or derivative thereof.

133. The composition of claim 1 wherein the drug is a raf kinase inhibitor or analogue or derivative thereof.

134. The composition of claim 1 wherein the drug is a lysylhydroxylase inhibitor or an analogue or derivative thereof.

135. The composition of claim 1 wherein the drug is an IKK 1/2 inhibitor or an analogue or derivative thereof.

136. The composition of claim 74 wherein the drug is a cytokine modulator.

137. The composition of claim 74 wherein the drug is a cytokine antagonist.

138. The composition of claim 1 wherein the drug is water-insoluble.

139. The composition of claim 1 in anhydrous form.

140. The composition of claim 1 in sterile form.

141. The composition of claim 1 wherein the polymer contributes about 0.5-40 percent of the weight of the composition.

142. The composition of claim 1 further comprising a solvent.

143. The composition of claim 142 wherein the solvent comprises water.

144. The composition of claim 1 further comprising a buffer.

145. The composition of claim 144 wherein the buffer maintains the pH of the composition within the range of 4-10.

146. The composition of claim 144 wherein the buffer maintains the pH of the composition within the range of 5-9.

147. The composition of claim 144 wherein the buffer maintains the pH of the composition within the range of 6-8.

148. The composition of claim 144 wherein the buffer comprises phosphate.

149. The composition of claim 1 further comprising protein.

150. The composition of claim 149 wherein the protein is collagen.

151. The composition of claim 149 wherein the protein contains primary amino groups.

152. The composition of claim 1 further comprising polysaccharide.

153. The composition of claim 152 wherein the polysaccharide is glysoaminoglycan.

154. A method of affecting biological processes in vivo comprising:
a) selecting an in vivo biological tissue comprising functional groups X;
b) providing a composition comprising a synthetic polymer and a drug, the polymer comprising multiple activated groups Y, where Y is reactive with X;
c) contacting the tissue of step a) with the composition of step b) under conditions where i) X reacts with Y and ii) biological processes in the vicinity of the tissue are affected by the drug.

155. The method of claim 154 wherein the biological tissue has undergone surgical trauma prior to being contacted with the composition of step b), thereby placing the tissue at risk of adhesion formation.

156. The method of claim 155 wherein the adhesion formation is an undesired by-product of abdominal surgery.

157. The method of claim 155 wherein the adhesion formation is an undesired by-product of cardiac surgery.

158. The method of claim 155 wherein the adhesion formation is an undesired by-product of spinal surgery.

159. The method of claim 155 wherein the adhesion formation is an undesired by-product of nasal surgery.

160. The method of claim 155 wherein the adhesion formation is an undesired by-product of throat surgery.

161. The method of claim 155 wherein the adhesion formation is an undesired by-product of breast implant.

162. The method of claim 155 wherein the biological tissue has undergone surgical trauma prior to being contacted with the composition of step b), the surgery being performed to excise tumor.

163. The method of claim 162 wherein the surgery is breast surgery.

164. The method of claim 162 wherein the surgery is breast tumor lumpectomy.

165. The method of claim 162 wherein the surgery is brain surgery.

166. The method of claim 162 wherein the surgery is hepatic resection surgery.

167. The method of claim 162 wherein the surgery is colon tumor resection surgery.

168. The method of claim 162 wherein the surgery is neurosurgical tumor resection.

169. The method of claim 154 wherein tissue is the interior surface of a physiological lumen.

170. The method of claim 169 wherein the tissue is a blood vessel.

171. The method of claim 169 wherein the tissue is a Fallopian tube.

172. The method of claim 169 wherein the tissue has undergone balloon catheterization.

173. A method comprising:
a) contacting tissue in vivo with a synthetic polymer comprising multiple activated groups, where the activated groups are tissue-reactive;
b) reacting the synthetic polymer with the tissue so as to covalently adhere the synthetic polymer to the tissue.

174. The method of claim 173 wherein tissue is a blood vessel.

175. The method of claim 173 wherein the tissue is prone to restenosis.

176. The method of claim 173 wherein adhesion of the tissue to secondary tissue is mitigated upon reacting the synthetic polymer with the tissue.

177. The method of claim 173 wherein the tissue does not react with any other synthetic polymer.

178. The method of claim 173 wherein the synthetic polymer is not in admixture with any other polymer that is reactive with the synthetic polymer.

179. The method of claim 173 wherein the synthetic polymer is not in admixture with any other polymer that is reactive with the tissue.

180. The method of claim 173 wherein the synthetic polymer comprises alkylene oxide residues.

181. The method of claim 173 wherein the synthetic polymer is a 4-arm PEG.

182. The method of claim 173 wherein the synthetic polymer comprises a plurality of thiol-reactive groups.

183. The method of claim 173 wherein the synthetic polymer comprises a plurality of hydroxyl-reactive groups.

184. The method of claim 173 wherein the synthetic polymer comprises a plurality of amine-reactive groups.

185. A method comprising:
a) contacting a non-living surface with a synthetic polymer comprising multiple activated groups, where the activated groups are tissue-reactive;
b) reacting the synthetic polymer with the surface so as to covalently adhere the synthetic polymer to the surface.

186. The method of claim 185 wherein the surface is a surface of a catheter.

187. The method of claim 185 wherein the surface is a surface of a contact lens.

188. The method of claim 185 wherein adhesion of the surface to living tissue is mitigated upon reacting the synthetic polymer with the surface.

189. The method of claim 185 wherein the surface is not reacted with any other synthetic polymer.

190. The method of claim 185 wherein the synthetic polymer is not in admixture with any other polymer that is reactive with the synthetic polymer.

191. The method of claim 185 wherein the synthetic polymer is not in admixture with any other polymer that is reactive with the surface.

192. The method of claim 185 wherein the synthetic polymer comprises alkylene oxide residues.

193. The method of claim 185 wherein the synthetic polymer is a 4-arm PEG.

194. The method of claim 185 wherein the synthetic polymer comprises a plurality of thiol-reactive groups.

195. The method of claim 185 wherein the synthetic polymer comprises a plurality of hydroxyl-reactive groups.

196. The method of claim 185 wherein the synthetic polymer comprises a plurality of amine-reactive groups.

197. A method for preparing a reactive composition, the method comprising:
a) providing a synthetic polymer comprising multiple activated groups;
b) combining the synthetic polymer with a buffer having a pH of less than 6 to form a homogeneous solution; and
c) raising the pH of the homogeneous solution to a pH of more than about 7.8, thereby rendering the synthethic polymer reactive.

198. The method of claim 197 wherein the synthetic polymer comprises alkylene oxide residues.

199. The method of claim 197 wherein the synthetic polymer comprises thiol-reactive groups.

200. The method of claim 198 wherein the synthetic polymer comprises N-oxysuccinimidyl groups.

201. The method of claim 198 wherein the synthetic polymer is combined with a drug.

202. The method of claim 201 wherein the drug is hydrophobic.

203. The method of claim 202 wherein the drug is in association with a secondary carrier, and the secondary carrier is dispersed in aqueous media.

204. A method of adhering a synthetic polymer to in vivo tissue, the method comprising:
a) providing a synthetic polymer comprising multiple activated groups;
b) combining the synthetic polymer with a buffer having a pH of less than 6 to form a homogeneous solution;
c) raising the pH of the homogeneous solution to a pH of more than about 7.8, thereby rendering the synthethic polymer reactive; and
d) contacting the reactive synthetic polymer with in vivo tissue.

205. The method of claim 204 wherein the synthetic polymer comprises alkylene oxide residues.

206. The method of claim 204 wherein the synthetic polymer comprises thiol-reactive groups.

207. The method of claim 204 wherein the synthetic polymer comprises N-oxysuccinimidyl groups.

208. The method of claim 204 wherein the synthetic polymer is contacted with the tissue prior to raising the pH of the homogeneous solution to a pH of more than about 7.8.

209. The method of claim 204 wherein the synthetic polymer is contacted with the tissue after raising the pH of the homogeneous solution to a pH of more than about 7.8.

210. The method of claim 204 wherein the synthetic polymer is combined with a drug.

211. The method of claim 210 wherein the drug is hydrophobic.

212. The method of claim 211 wherein the drug is in association with a secondary carrier, and the secondary carrier is dispersed in aqueous media.

213. A composition comprising:
a) a synthetic polymer comprising multiple activated groups; and
b) an aqueous buffer;
wherein the composition is a homogeneous solution having a pH of less than 6.

214. A composition comprising:
a) a synthetic polymer comprising multiple activated groups; and
b) an aqueous buffer;
wherein the composition is a homogeneous solution having a pH of greater than about 7.8.

215. The compositions of claims 213 or 214 wherein the composition does not contain any polymer that is reactive with the synthetic polymer.

216. The compositions of claims 213 or 214 wherein the composition further comprises a drug.

217. The compositions of claims 213 or 214 wherein the composition further comprises a hydrophobic drug.

218. The compositions of claims 213 or 214 wherein the composition further comprises a hydrophobic drug is association with a secondary carrier.

219. The compositions of claims 213 or 214 wherein the secondary carrier is in the form of a micelle or nanosphere.

220. The compositions of claims 213 or 214 wherein the synthetic polymer comprises alkylene oxide residues.

221. The compositions of claims 213 or 214 wherein the synthetic polymer comprises thiol-reactive groups.

222. The compositions of claims 213 or 214 wherein the synthetic polymer comprises N-oxysuccinimidyl groups.

223. The compositions of claims 213 or 214 wherein the synthetic polymer is a 4-arm PEG.

224. The compositions of claims 213 or 214 in sterile form.

225. A method of coating a device comprising:
(a) applying a multifunctional hydroxysuccinimidyl PEG derivative to the surface of the device; and
(b) allowing the derivative to react with functional groups on the device surface.

226. The method of claim 225 wherein the functional surface groups on the device are incorporated into the device using a surface treatment process.

227. The method of claim 226 wherein the surface treatment process is a plasma treatment process.

228. The method of claim 226 wherein the surface treatment process comprises coating the surface of the device with a polymer, wherein the polymer comprises functional groups that can react with the mulitfunctional hydroxysuccinimidyl PEG derivative.

229. The method of claim 228 wherein the polymer comprises amino groups.

230. The method of claim 229 wherein the polymer is chitosan.

231. The method of claim 229 wherein the polymer is polyethyleneimine.

232. The method of claim 225 wherein the multifunctional hydroxysuccinimidyl PEG derivative is tetra functional poly(ethylene glycol) succinimidyl glutarate.

233. A method of reducing surgical adhesions comprising applying a multifunctional hydroxysuccinimidyl PEG derivative to a tissue surface.

234. The method of claim 233 wherein the multifunctional hydroxysuccinimidyl PEG derivative is in the form of a solution, wherein the solution has a basic pH

235. The method of claim 234 wherein the pH is greater than 8.

236. The method of claim 235 wherein the multifunctional hydroxysuccinimidyl PEG derivative is tetra functional poly(ethylene glycol) succinimidyl glutarate.

237. The method of claim 233 wherein the multifunctional hydroxysuccinimidyl PEG derivative is not in admixture with any other tissue reactive compound.

238. The method of claim 233 wherein the multifunctional hydroxysuccinimidyl PEG derivative is not in admixture with any component that will react with the derivative.

239. A method of reducing surgical adhesions comprising applying a tissue reactive composition consisting essentially of a multifunctional hydroxysuccinimidyl PEG derivative to a tissue surface.

240. A method of reducing surgical adhesions comprising applying a tissue reactive composition consisting of a multifunctional hydroxysuccinimidyl PEG derivative to a tissue surface.
